(19) United States
(12) Patent Application Publication
(10) Pub. No.: US 2023/0129061 A1
(43) Pub. Date: Apr. 27, 2023

(54) The use of growth factor-encoding nucleoside-modified mRNA for periodontal tissue regeneration

(71) Applicants: The Trustees of the University of Pennsylvania, Philadelphia, PA (US); Chulalongkorn University, Bangkok (TH)

(72) Inventors: Drew Weissman, Wynnewood, PA (US); Norbert Pardi, Philadelphia, PA (US); Rangsini Mahanonda, Bangkok (TH); Sathit Pichyangkul, Bangkok (TH); Chantrakorn Champaiboon, Bangkok (TH); Wichaya Wisitrasameewong, Bangkok (TH); Noppadol Sa-Ard-Iam, Bangkok (TH)

(21) Appl. No.: 17/759,361

(22) PCT Filed: Jan. 22, 2021
(86) PCT No.: PCT/US2021/014614

§ 371 Date: Jul. 22, 2022

Related U.S. Application Data
(60) Provisional application No. 62/965,235, filed on Jan. 24, 2020.

(51) Int. Cl. A61K 48/00; A61P 43/00; A61K 9/51

(52) U.S. Cl. CPC A61K 48/005; A61P 43/00; A61K 9/5123

(57) ABSTRACT

The present disclosure relates to compositions and methods for inducing regeneration of periodontal tissue and bone in a subject. In certain aspects, the composition comprises at least one isolated RNA encoding at least one growth factor, or fragment or variant thereof. In one aspect, the composition comprises a nucleoside-modified RNA encoding at least one growth factor, or fragment or variant thereof.

### CROSS-REFERENCES TO RELATED APPLICATIONS

This application claims priority to U.S. Provisional Patent Application No. 62/965,235 filed on Jan. 24, 2020, the contents of which are incorporated by reference herein in its entirety.

### BACKGROUND OF THE INVENTION

The Global Burden of Disease Study (2010) ranked severe periodontitis as the sixth-most prevalent disease in the world, affecting 11.2% (743 million people) worldwide. Severe periodontitis has been recognized as the major oral health problem among adults (age 35-59) and particularly in elderly people (age 60 and over). Between 2015 and 2030, the number of the elderly people in the world is projected to grow by 56% from 901 million to more than 1.4 billion (United Nation, 2015), suggesting the prevalence of severe periodontitis would be markedly increased.

Periodontitis is a chronic inflammatory disease in response to bacterial plaque. The disease causes damaging inflammation and destroys tooth supporting tissues, called periodontium which consists of gingiva, bone, periodontal ligament and cementum. Severe periodontitis is the primary cause of tooth loss and it profoundly affects oral health and its functions, including eating, speaking, aesthetics, and quality of life. The disease accounts for large amount of healthcare costs and socio-economic impacts. So far, there has been no therapeutic approach that effectively regenerates periodontal tissue. Human recombinant platelet-derived growth factor BB (PDGF-BB) has been the only human recombinant protein approved by the U.S. Food and Drug administration (FDA) when combined with synthetic calcium phosphate matrix for periodontal regeneration (GEM 21STM, Osteohealth, USA). Another human recombinant protein, bone morphogenetic protein-2 (BMP-2) when combined with absorbable collagen sponge has been approved by U.S. FDA as a bone graft for certain dental bone regenerative procedures (INFUSE®, Medtronic, USA). However, due to short half-life of proteins and high cost, these products have not been widely used.

In addition to bone destruction around tooth caused by periodontal diseases, such circumstance can be found around dental implant (peri-implant diseases) and in edentulous ridge after tooth extraction, traumatic injury or other diseases. Bone regeneration is, thus, often required to reconstruct such bone deficiencies.

Thus, there is a need in the art for improved compositions and methods for treating and preventing periodontitis and bone defects. This invention satisfies this unmet need.

### SUMMARY OF THE INVENTION

In one aspect, the present invention relates to a composition for inducing regeneration of periodontal tissue, bone, or a combination thereof in a subject, the composition comprising at least one isolated RNA encoding at least one growth factor, or fragment or variant thereof.

In one embodiment, the at least one isolated RNA of the composition comprises at least one isolated nucleoside-modified RNA. In one embodiment, the at least one isolated nucleoside-modified RNA comprises pseudouridine. In one embodiment, the at least one isolated nucleoside-modified RNA comprises 1-methyl-pseudouridine. In one embodiment, the at least one isolated nucleoside-modified RNA is a purified nucleoside-modified RNA.

In one embodiment, the at least one growth factor of the composition comprises PDGF-BB or BMP-2.

In one embodiment, the composition further comprises a lipid nanoparticle (LNP). In one embodiment, the at least one isolated RNA is encapsulated within the LNP. In one embodiment, the composition comprises a scaffold.

In one aspect, the present invention relates to a method of inducing regeneration of periodontal tissue, bone, or a combination thereof in a subject comprising administering to the subject an effective amount of a composition comprising at least one isolated RNA encoding at least one growth factor, or fragment or variant thereof.

In one embodiment, the at least one isolated RNA comprises at least one isolated nucleoside-modified RNA. In one embodiment, the at least one isolated nucleoside-modified RNA comprises pseudouridine. In one embodiment, the at least one isolated nucleoside-modified RNA comprises 1-methyl-pseudouridine. In one embodiment, the at least one isolated nucleoside-modified RNA is a purified nucleoside-modified RNA.

In one embodiment, the at least one growth factor comprises PDGF-BB or BMP-2.

In one embodiment, the composition further comprises a lipid nanoparticle (LNP). In one embodiment, the at least one isolated RNA is encapsulated within the LNP. In one embodiment, the composition comprises a scaffold.

In one embodiment, the method comprises one or more administration of the composition of the present invention. In one embodiment, the method comprises a single administration of the composition. In one embodiment, the method comprises multiple administrations of the composition. In one embodiment, the method comprises administration of the scaffold to periodontal tissue, bone defects, or a combination thereof of the subject.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A and FIG. 1B depict the results of example experiments examining the production of PDGF-BB protein. Human periodontal ligament cells (PDLCs) at 100,000 cells/well were transfected with 2 μg of PDGF-BB mRNA in Lipofectamine 2000. Culture supernatants were harvested and analyzed by ELISA. FIG. 1A; The mean concentration of intracellular PDGF-BB at 24 h. FIG. 1B; The mean concentration of extracellular PDGF-BB secretion at 24, 48, and 72 h. Data shown are mean±SE, (n=3).

FIG. 2 depicts the results of example experiments depicting the mean percentage of periodontal cell viability at 24, 48 and 72 hours. Human PDLCs (100,000 cells/well) were transfected with 2 μg of PDGF-BB mRNA in Lipofectamine 2000. At each time-point, cell viability of PDLCs was assessed by alamarBlue assay. Data shown were mean±SE (n=3).

FIG. 3 depicts the results of example experiments depicting the biological function of PDGF-BB protein translated from mRNA in vitro. PDGF-BB produced from mRNA were assessed for cell proliferation. Data shown are mean±SE (n=3).

FIG. 4A and FIG. 4B depict the results of example experiments of an in vitro study of PDGF-BB expression in transfected HEK 293T. Cells at 200,000 cells/well were transfected with varying concentrations (0.2, 0.6, and 2 μg) of pseudouridine (Ψ) (TriLink) and N1-methylpseudouridine (m1Ψ) modified PDGF-BB mRNA complexed with lipofectamine 2000. Production of protein PDGF-BB in culture supernatants at 48 h after transfection was assessed by ELISA (FIG. 4A). Cell viability was analyzed by alamarBlue assay (FIG. 4B). Data shown are mean±SE (n=3).

FIG. 5A through FIG. 5D depict the results of example experiments of in vitro secretion of PDGF-BB protein in clinically relevant target cells after transfection with PDGF-BB mRNA formulated with different vehicles. Human PDLCs (FIG. 5A) and gingival fibroblasts (GFs) (FIG. 5B) at 100,000 cells/well were transfected with 2 of PDGF-BB mRNA in dPBS, sucrose citrate buffer, lipofectamine 2000 and LNP. After 48 h of cell transfection, culture supernatants were harvested and measured for PDGF-BB by ELISA. Cell viability of PDLCs (FIG. 5C) and GFs (FIG. 5D) was assessed by alamarBlue assay. Data shown are mean±SE (n=4).

FIG. 6A through FIG. 6D depict the results of example experiments examining the biological function of PDGF-BB protein translated from mRNA. (FIG. 6A and FIG. 6B) Transfection of PDLCs and GFs with PDGF mRNA led to endogenous production of vascular endothelial growth factor-A (VEGF-A) from both target cells, supporting previous studies that PDGF-BB can induce expression of VEGF-A. PDGF-BB secreted from mRNA transfected cells was compared with the same concentration of recombinant PDGF-BB (5 ng/ml) in cell migration assay (FIG. 6C) and endothelial cell tube formation assay (FIG. 6D). Data shown in (FIG. 6A) and (FIG. 6B) are mean±SE (n=4). Data shown in (FIG. 6C) are representative of 3 experiments and (FIG. 6D) are results from one experiment.

FIG. 7A through FIG. 7C depict the results of example experiments of in vivo vehicle optimization of PDGF-BB mRNA in rat gingiva. (FIG. 7A) PDGF-BB mRNA formulated with different vehicles (dPBS, sucrose citrate buffer, lipofectamine 2000 and LNP were injected into rat gingiva (palate) (total 30 μg; 6 sites, 5 μg/6 μl/site). Whole gingival tissues of the palate were harvested at 24 h after injection, digested with RIPA+proteinase inhibitor and measured for PDGF-BB protein production by ELISA. (FIG. 7B) Time course of PDGF-BB protein production after PDGF-BB mRNA-LNP injection into rat gingiva. PDGF-BB mRNA-LNP was injected into rat gingiva (palate) (total 30 μg; 6 sites, 5 μg/6 μl/site) and the whole gingival tissues of the palate were harvested at 5, 24, 48 and 72 h and then assessed for PDGF-BB production by ELISA. (FIG. 7C) No sign of gingival inflammation after injection with PDGF-BB mRNA-LNP was observed. The amount of TNF-α in rat gingiva was less than 100 pg/mg of protein at all time points (5-72 h) and the amount of IL-6 was undetected (data not shown). Data shown in FIG. 7A and FIG. 7B are mean±SE (n=4).

FIG. 8A and FIG. 8B depict the results of example experiments depicting in vitro production of BMP-2 protein in clinically relevant target cells after transfection with m1Ψ-BMP-2 mRNA. Human PDLCs at 100,000 cells/well were transfected with 2 μg of BMP-2 mRNA in Lipofectamine 2000. (FIG. 8A) After 24 h of cell transfection, cells were harvested and measured for BMP-2 by ELISA. (FIG. 8B) After 24, 48 and 72 h of cell transfection, culture supernatants were harvested and analyzed by ELISA. Data shown are mean±SE (n=3).

FIG. 9 depicts the results of example experiments investigating cell viability after transfection with m1Ψ-BMP-2 mRNA. Human PDLCs at 100,000 cells/well were transfected with 2 μg of BMP-2 mRNA in Lipofectamine 2000. At each time-point of observation, cell viability of PDLCs was assessed by almarBlue assay. Data shown are mean±SE (n=3).

FIG. 10 depicts the results of example experiments investigating the biological function of BMP-2 protein translated from mRNA in vitro. BMP-2 produced from mRNA were assessed for cell proliferation. Data shown are mean (n=3).

FIG. 11 depicts qualitative results of example experiments investigating bone regeneration of rat calvarial bone defects via PDGF-BB mRNA in LNP with collagen scaffold. Shown are representative three dimensionally reconstructed radiographic images of rat calvarial defects at week 4 after treatment for each treatment condition.

FIG. 12 depicts quantitative results of example experiments investigating bone regeneration of rat calvarial bone defects via PDGF-BB mRNA in LNP with collagen scaffold. Measurements of bone volume (mm³) in rat calvarial defects at week 4 post treatment were collected. Data represent the mean of two defects from one animal per treatment group.

FIG. 13 depicts qualitative results of example experiments investigating bone regeneration of rat calvarial bone defects via BMP-2 mRNA in LNP with collagen scaffold. Shown are representative three dimensionally reconstructed radiographic images of rat calvarial defects at week 4 after treatment for each treatment condition.

FIG. 14 depicts quantitative results of example experiments investigating bone regeneration of rat calvarial bone defects via BMP-2 mRNA in LNP with collagen scaffold. Measurements of bone volume (mm³) in rat calvarial defects at week 4 post treatment were collected. Data represent the mean of two defects from one animal per treatment group.

FIG. 15A and FIG. 15B depict results of example experiments investigating in vivo protein expression in large animals (dogs) after gingival injection of PDGF-BB mRNA in LNP. PDGF-BB mRNA formulated with LNP or dPBS (control) were injected into dog gingiva on the buccal side (total of 40 μg mRNA by 8 injections per tooth, 5 μg/μl of mRNA per injection). Buccal tissue biopsies (approximately 2 mm×2 cm) were collected after injection at 24 h (n=6), 48 h (n=6), and 72 h (n=4) and assessed for PDGF-BB protein production (FIG. 15A). Data are shown as mean±SE of PDGF-BB. FIG. 15B depicts the gingival injection site and demonstrates that no sign of gingival inflammation was observed until day 3.

### DETAILED DESCRIPTION

The present invention relates to compositions and methods for regenerating periodontal tissue and bone in a subject. In some embodiments, the invention provides a composition comprising at least one nucleoside-modified RNA encoding at least one growth factor. In some embodiments, the at least one nucleoside-modified RNA encodes platelet-derived growth factor BB (PDGF-BB), bone morphogenetic protein-2 (BMP-2), or a combination thereof. In some embodiments, the at least one nucleoside-modified RNA is encapsulated in a lipid nanoparticle (LNP). In some embodiments, the at least one nucleoside-modified RNA is contained within a scaffold, including but not limited to a hydrogel, electrospun scaffold, or the like.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as, a human.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translated by translational machinery in a cell. Exemplary modified nucleosides are described elsewhere herein. For example, an mRNA where some or all of the uridines have been replaced with pseudouridine, 1-methyl psuedouridine, or another modified nucleoside, such as those described elsewhere herein. In some embodiments, the nucleotide sequence may contain a sequence where some or all cytodines are replaced with methylated cytidine, or another modified nucleoside, such as those described elsewhere herein.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The terms “patient,” “subject,” “individual,” and the like are used interchangeably herein, and refer to any animal, or cells thereof whether in vitro or in situ, amenable to the methods described herein. In some non-limiting embodiments, the patient, subject or individual is a mammal. In some non-limiting embodiments, the patient, subject or individual is a canine. In some non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In some instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

In some embodiments, “pseudouridine” refers to m¹acp³Ψ (1-methyl (3-amino-3-carboxypropyl) pseudouridine). In another embodiment, the term refers to m¹Ψ (1-methylpseudouridine). In another embodiment, the term refers to Ψm (2′-O-methylpseudouridine. In another embodiment, the term refers to m⁵D (5-methyldihydrouridine). In another embodiment, the term refers to m³Ψ (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. By way of one non-limiting example, a promoter that is recognized by bacteriophage RNA polymerase and is used to generate the mRNA by in vitro transcription.

The term “therapeutic” as used herein means a treatment and/or prophylaxis. A therapeutic effect is obtained by suppression, diminution, remission, prevention, or eradication of at least one sign or symptom of a disease or disorder.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for promoting or inducing the regeneration of periodontal tissue and bone in a subject. For example, in some embodiments, the compositions and methods described herein are useful for preventing and/or treating periodontitis and/or bone defects.

In some embodiments, the present invention provides a composition comprising a nucleic acid molecule encoding a growth factor, where the growth factor induces regeneration of periodontal tissue and/or bone in the subject. In one embodiment, the composition of the invention comprises in vitro transcribed (IVT) RNA. For example, in some embodiments, the composition of the invention comprises IVT RNA which encodes a growth factor, where the growth factor induces regeneration of periodontal tissue and/or bone in the subject.

Exemplary growth factors that can be encoded and used to regenerate periodontal tissue and/or bone include, but are not limited to, PDGF-BB, PDGF-AA, PDGF-AB, BMP-2, FGF, IGF, VEGF, and EGF.

In some embodiments, the growth factor-encoding nucleic acid of the present composition is a nucleoside-modified RNA. The present invention is based in part on the finding that nucleoside-modified RNA encoding a growth factor (e.g., PDGF-BB and BMP-2) can induce periodontal tissue regeneration, reduce gingival inflammation, and promote bone generation.

In some embodiments, the growth factor-encoding nucleic acid of the present composition is a purified nucleoside-modified RNA. For example, in some embodiments, the composition is purified such that it is free of double-stranded contaminants.

In some embodiments, the composition comprises a lipid nanoparticle (LNP). For example, in one embodiment, the composition comprises a growth factor-encoding nucleic acid molecule encapsulated within a LNP. In some instances, the LNP enhances cellular uptake of the nucleic acid molecule.

In some embodiments, the composition comprises a scaffold, such as a tissue engineering scaffold, comprising the growth factor-encoding nucleic acid molecule. For example, in one embodiment, the scaffold comprises LNP encapsulating the growth factor-encoding nucleic acid molecule. In one embodiment, the scaffold comprises a cell or cell population comprising the growth factor-encoding nucleic acid molecule. In some embodiments, the scaffold comprises a hydrogel, electrospun scaffold or the like comprising a biopolymer, synthetic polymer or combination thereof.

In one embodiment, the present invention provides a method for promoting or inducing regeneration of periodontal tissue and/or bone in a subject. In one embodiment, the present invention provides a method for treating or preventing gum disease or periodontitis in a subject. In one embodiment, the present invention provides a method for treating or preventing bone defects in a subject. In one embodiment, the present invention provides a method for treating or preventing gum disease, periodontitis, or bone defects in a subject. In some embodiments, the method comprises administering to the subject a composition comprising one or more nucleoside-modified RNA encoding one or more growth factors.

In some embodiments, the method comprises administering a plurality of doses to the subject. In another embodiment, the method comprises administering a single dose of the composition, where the single dose is effective in inducing regeneration of periodontal tissue and/or bone in a subject. In one embodiment, the method comprises implanting a scaffold comprising the one or more nucleoside-modified RNA encoding one or more growth factors to a subject. In one embodiment, the method provides a sustained or prolonged response. In one embodiment, the method comprises administering one or more compositions, as described herein, to the gum, jaw, mouth or other parts of the body of the subject.

### Growth Factor

The present invention provides a composition that induces regeneration of periodontal tissue and/or bone in a subject.

In one embodiment, the composition comprises a growth factor, or fragment or variant thereof. In one embodiment, the growth factor comprises a polypeptide or peptide, where the growth factor induces regeneration of periodontal tissue and/or bone. In one embodiment, the composition comprises a fragment or variant of a growth factor, where the fragment or variant of the growth factor retains the ability to induce regeneration of periodontal tissue and/or bone.

In one embodiment, the composition comprises a nucleic acid sequence, which encodes a growth factor, or a fragment or variant thereof. For example, in some embodiments, the composition comprises a nucleoside-modified RNA encoding a growth factor, or a fragment or variant thereof. In some embodiments, the composition comprises a purified, nucleoside-modified RNA encoding a growth factor, or a fragment or variant thereof.

Exemplary growth factors that can promote or induce the regeneration of periodontal tissue and/or bone include, but is not limited to, PDGF-BB and BMP-2.

In one embodiment, the composition comprises a nucleoside-modified RNA comprising a nucleic acid sequence encoding PDGF-BB comprising an amino acid sequence comprising at SEQ ID NO: 1, or a fragment or variant thereof, wherein the nucleic acid sequence comprises at least one modified nucleoside.

In one embodiment, the amino acid sequence of human PDGF-BB comprises:

(SEQ ID NO: 1)
MNRCWALFLSLCCYLRLVSAEGDPIPEELYEMLSD
HSIRSFDDLQRLLHGDPGEEDGAELDLNMTRSHSG
GELESLARGRRSLGSLTIAEPAMIAECKTRTEVFE
ISRRLIDRTNANFLVWPPCVEVQRCSGCCNNRNVQ
CRPTQVQLRPVQVRKIEIVRKKPIFKKATVTLEDH
LACKCETVAAARPVTRSPGGSQEQRAKTPQTRVTI
RTVRVRRPPKGKHRKFKHTHDKTALKETLGA

In one embodiment, the composition comprises a nucleoside-modified RNA comprising a nucleic acid sequence encoding BMP-2 comprising an amino acid sequence comprising at SEQ ID NO: 2, or a fragment or variant thereof, wherein the nucleic acid sequence comprises at least one modified nucleoside.

In one embodiment, the amino acid sequence BMP-2 comprises:

(SEQ ID NO: 2)
MVAGTRCLLALLLPQVLLGGAAGLVPELGRRKFAA
ASSGRPSSQPSDEVLSEFELRLLSMFGLKQRPTPS
RDAVVPPYMLDLYRRHSGQPGSPAPDHRLERAASR
ANTVRSFHHEESLEELPETSGKTTRRFFFNLSSIP
TEEFITSAELQVFREQMQDALGNNSSFHHRINIYE
IIKPATANSKFPVTRLLDTRLVNQNASRWESFDVT
PAVMRWTAQGHANHGFVVEVAHLEEKQGVSKRHVR
ISRSLHQDEHSWSQIRPLLVTFGHDGKGHPLHKRE
KRQAKHKQRKRLKSSCKRHPLYVDFSDVGWNDWIV
APPGYHAFYCHGECPFPLADHLNSTNHAIVQTLVN
SVNSKIPKACCVPTELSAISMLYLDENEKVVLKNY
QDMVVEGCGCR

In one embodiment, the composition comprises a nucleoside-modified RNA comprising a nucleic acid sequence encoding PDGF-BB, wherein the nucleic acid sequence is encoded by a DNA sequence comprising SEQ ID NO: 3, or a fragment or variant thereof, wherein the nucleic acid sequence comprises at least one modified nucleoside.

In certain embodiments, the nucleoside-modified RNA encoding PDGF-BB is encoded by an optimized DNA sequence. In one embodiment, the DNA sequence encoding the nucleoside-modified RNA encoding PDGF-BB, comprises the nucleotide sequence of:

(SEQ ID NO: 3)
ATGAACCGCTGCTGGGCCCTGTTCCTGTCCCTGTG
CTGCTACCTGCGCCTGGTGTCCGCCGAGGGCGACC
CCATCCCCGAGGAGCTGTACGAGATGCTGTCCGAC
CACTCCATCCGCTCCTTCGACGACCTGCAGCGCCT
GCTGCACGGCGACCCCGGCGAGGAGGACGGCGCCG
AGCTGGACCTGAACATGACCCGCTCCCACTCCGGC
GGCGAGCTGGAGTCCCTGGCCCGCGGCCGCCGCTC
CCTGGGCTCCCTGACCATCGCCGAGCCCGCCATGA
TCGCCGAGTGCAAGACCCGCACCGAGGTGTTCGAG
ATCTCCCGCCGCCTGATCGACCGCACCAACGCCAA
CTTCCTGGTGTGGCCCCCCTGCGTGGAGGTGCAGC
GgTGCTCCGGCTGCTGCAACAACCGCAACGTGCAGT
GCCGCCCCACCCAGGTGCAGCTGCGCCCCGTGCAGG
TGCGCAAGATCGAGATCGTGCGCAAGAAGCCCATC
TTCAAGAAGGCCACCGTGACCCTGGAGGACCACCT
GGCCTGCAAGTGCGAGACCGTGGCCGCCGCCCGCC
CCGTGACCCGCTCCCCCGGCGGCTCCCAGGAGCAG
CGCGCCAAGACCCCCCAGACCCGCGTGACCATCCG
CACCGTGCGCGTGCGCCGCCCCCCCAAGGGCAAGC
ACCGCAAGTTCAAGCACACCCACGACAAGACCGCC
CTGAAGGAGACCCTGGGCGCC

In one embodiment, the composition comprises a nucleoside-modified RNA comprising a nucleic acid sequence encoding BMP-2, wherein the nucleic acid sequence is encoded by a DNA sequence comprising SEQ ID NO: 4, or a fragment or variant thereof, wherein the nucleic acid sequence comprises at least one modified nucleoside.

In certain embodiments, the nucleoside-modified RNA encoding BMP-2 is encoded by an optimized DNA sequence. In one embodiment, the DNA sequence encoding the nucleoside-modified RNA encoding BMP-2, comprises the nucleotide sequence of:

(SEQ ID NO: 4)
ATGGTGGCCGGCACCCGCTGCCTGCTGGCCCTGCT
GCTGCCCCAGGTGCTGCTGGGCGGCGCCGCCGGCC
TGGTGCCCGAGCTGGGCCGCCGCAAGTTCGCCGCC
GCCTCCTCCGGCCGCCCCTCCTCCCAGCCCTCCGA
CGAGGTGCTGTCCGAGTTCGAGCTGCGCCTGCTGT
CCATGTTCGGCCTGAAGCAGCGCCCCACCCCCTCC
CGCGACGCCGTGGTGCCCCCCTACATGCTGGACCT
GTACCGCCGCCACTCCGGCCAGCCCGGCTCCCCCG
CCCCCGACCACCGCCTGGAGCGCGCCGCCTCCCGC
GCCAACACCGTGCGCTCCTTCCACCACGAGGAGTC
CCTGGAGGAGCTGCCCGAGACCTCCGGCAAGACCA
CCCGCCGCTTCTTCTTCAACCTGTCCTCCATCCCC
ACCGAGGAGTTCATCACCTCCGCCGAGCTGCAGGT
GTTCCGCGAGCAGATGCAGGACGCCCTGGGCAACA
ACTCCTCCTTCCACCACCGCATCAACATCTACGAG
ATCATCAAGCCCGCCACCGCCAACTCCAAGTTCCC
CGTGACCCGCCTGCTGGACACCCGCCTGGTGAACC
AGAACGCCTCCCGCTGGGAGTCCTTCGACGTGACC
CCCGCCGTGATGCGCTGGACCGCCCAGGGCCACGC
CAACCACGGCTTCGTGGTGGAGGTGGCCCACCTGG
AGGAGAAGCAGGGCGTGTCCAAGCGCCACGTGCGC
ATCTCCCGCTCCCTGCACCAGGACGAGCACTCCTG
GTCCCAGATCCGCCCCCTGCTGGTGACCTTCGGCC
ACGACGGCAAGGGCCACCCCCTGCACAAGCGCGAG
AAGCGCCAGGCCAAGCACAAGCAGCGCAAGCGCCT
GAAGTCCTCCTGCAAGCGCCACCCCCTGTACGTGG
ACTTCTCCGACGTGGGCTGGAACGACTGGATCGTG
GCCCCCCCCGGCTACCACGCCTTCTACTGCCACGG
CGAGTGCCCCTTCCCCCTGGCCGACCACCTGAACT
CCACCAACCACGCCATCGTGCAGACCCTGGTGAAC
TCCGTGAACTCCAAGATCCCCAAGGCCTGCTGCGT
GCCCACCGAGCTGTCCGCCATCTCCATGCTGTACC
TGGACGAGAACGAGAAGGTGGTGCTGAAGAACTAC
CAGGACATGGTGGTGGAGGGCTGCGGCTGCCGC

In some embodiments, the growth factor comprises an amino acid sequence that is substantially homologous to the amino acid sequence of a growth factor described herein and retains the function of the original amino acid sequence. For example, in some embodiments, the amino acid sequence of the growth factor has a degree of identity with respect to the original amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In one embodiment, the growth factor is encoded by a nucleic acid sequence of a nucleic acid molecule. In some embodiments, the nucleic acid sequence comprises DNA, RNA, cDNA, viral DNA, a variant thereof, a fragment thereof, or a combination thereof. In one embodiment, the nucleic acid sequence comprises a modified nucleic acid sequence. For example, in one embodiment the growth factor-encoding nucleic acid sequence comprises nucleoside-modified RNA, as described in detail elsewhere herein. In some instances, the nucleic acid sequence comprises additional sequences that encode linker or tag sequences that are linked to the growth factor by a peptide bond.

### Nucleic Acids

In one embodiment, the invention includes a nucleic acid molecule encoding a growth factor, or a fragment or variant thereof. In one embodiment, the invention includes a nucleoside-modified nucleic acid molecule. In one embodiment, the nucleoside-modified nucleic acid molecule encodes a growth factor, or a fragment or variant thereof. In one embodiment, the nucleoside-modified nucleic acid molecule encodes a plurality of growth factors, or a fragments or variants thereof. In some embodiments, the nucleoside-modified nucleic acid molecule encodes a growth factor, or a fragment or variant thereof, where the growth factor, or a fragment or variant thereof induces regeneration of periodontal tissue and/or bone.

The nucleic acid molecule can be made using any methodology in the art, including, but not limited to, in vitro transcription, chemical synthesis, or the like.

The nucleotide sequences encoding a growth factor, or a fragment or variant thereof, as described herein, can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the present invention includes nucleotide sequences that are substantially homologous to the nucleotide sequences recited herein and encode a growth factor of interest, or a fragment or variant thereof.

As used herein, a nucleotide sequence is “substantially homologous” to any of the nucleotide sequences described herein when its nucleotide sequence has a degree of identity with respect to the original nucleotide sequence at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. A nucleotide sequence that is substantially homologous to a nucleotide sequence encoding a growth factor can typically be isolated from a producer organism of the growth factor based on the information contained in the nucleotide sequence by means of introducing conservative or non-conservative substitutions, for example. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art.

Further, the scope of the invention includes nucleotide sequences that encode amino acid sequences that are substantially homologous to the amino acid sequences recited herein and preserve the function of the original amino acid sequence.

As used herein, an amino acid sequence is “substantially homologous” to any of the amino acid sequences described herein when its amino acid sequence has a degree of identity with respect to the original amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. The identity between two amino acid sequences can be determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

In one embodiment, the invention relates to a construct, comprising a nucleotide sequence encoding a growth factor, or a fragment or variant thereof. In one embodiment, the construct comprises a plurality of nucleotide sequences encoding a plurality of a growth factors, or a fragments or variants thereof. For example, in some embodiments, the construct encodes 1 or more, 2 or more, 3 or more, 4 or more, 5 or more, or 10 or more growth factors, or a fragments or variants thereof. In one embodiment, the invention relates to a construct, comprising a nucleotide sequence encoding an adjuvant.

In one embodiment, the composition comprises a plurality of constructs, each construct encoding one or more a growth factors, or a fragments or variants thereof. In some embodiments, the composition comprises 1 or more, 2 or more, 3 or more, 4 or more, 5 or more, 6 or more, 7 or more, 8 or more, 9 or more, 10 or more, 15 or more, or 20 or more constructs. In one embodiment, the composition comprises a first construct, comprising a nucleotide sequence encoding a first growth factor; and a second construct, comprising a nucleotide sequence encoding a second growth factor.

In another particular embodiment, the construct is operatively bound to a translational control element. The construct can incorporate an operatively bound regulatory sequence for the expression of the nucleotide sequence of the invention, thus forming an expression cassette.

Vectors

The nucleic acid sequences coding for the growth factor, or a fragment or variant thereof can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically.

The nucleic acid can be cloned into a number of types of vectors. For example, the nucleic acid can be cloned into a vector including, but not limited to a plasmid, a phagemid, a phage derivative, an animal virus, a PCR-generated linear DNA sequence, and a cosmid. Vectors of particular interest include expression vectors, replication vectors, probe generation vectors, sequencing vectors and vectors optimized for in vitro transcription.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, carbohydrates, peptides, cationic polymers, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/RNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to a composition of the present invention, in order to confirm the presence of the mRNA sequence in the host cell, a variety of assays may be performed. Such assays include, for example, “molecular biological” assays well known to those of skill in the art, such as Northern blotting and RT-PCR; “biochemical” assays, such as detecting the presence or absence of a particular peptide, e.g., by immunogenic means (ELISAs and Western blots) or by assays described herein to identify agents falling within the scope of the invention.

In Vitro Transcribed RNA

In one embodiment, the composition of the invention comprises in vitro transcribed (IVT) RNA encoding a growth factor, or a fragment or variant thereof. In one embodiment, the composition of the invention comprises IVT RNA encoding a plurality of growth factors, or fragments or variants thereof.

In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In one embodiment, the desired template for in vitro transcription is a growth factor, or a fragment or variant thereof capable of inducing regeneration of periodontal tissue and/or bone.

In one embodiment, the DNA to be used for PCR contains an open reading frame. The DNA can be from a naturally occurring DNA sequence from the genome of an organism. In one embodiment, the DNA is a full-length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. In another embodiment, the DNA to be used for PCR is a gene from a pathogenic or commensal organism, including bacteria, viruses, parasites, and fungi. In another embodiment, the DNA to be used for PCR is from a pathogenic or commensal organism, including bacteria, viruses, parasites, and fungi, including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that induce regeneration of periodontal tissue in an organism. In some instances, the genes are useful for a short term treatment. In some instances, the genes have limited safety concerns regarding dosage of the expressed gene.

In various embodiments, a plasmid is used to generate a template for in vitro transcription of mRNA, which is used for transfection.

Chemical structures with the ability to promote stability and/or translation efficiency may also be used. In some embodiments, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability of mRNA in the cell. On a circular DNA template, for instance, plasmid DNA, RNA polymerase produces a long concatameric product, which is not suitable for expression in eukaryotic cells. The transcription of plasmid DNA linearized at the end of the 3′ UTR results in normal sized mRNA, which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003)).

The conventional method of integration of polyA/T stretches into a DNA template is molecular cloning. However, polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to mRNA molecules. In one embodiment, RNAs produced by the methods to include a 5′ cap1 structure. Such cap1 structure can be generated using Vaccinia capping enzyme and 2′-O-methyltransferase enzymes (CellScript, Madison, Wis.). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)). In some embodiments RNA of the invention is introduced to a cell with a method comprising the use of TransIT®-mRNA transfection Kit (Mirus, Madison Wis.), which, in some instances, provides high efficiency, low toxicity, transfection.

Nucleoside-Modified RNA

In one embodiment, the composition of the present invention comprises a nucleoside-modified nucleic acid encoding a growth factor, or a fragment or variant thereof as described herein. In one embodiment, the composition of the present invention comprises a nucleoside-modified nucleic acid encoding a plurality of growth factors, or fragments or variants thereof.

For example, in one embodiment, the composition comprises a nucleoside-modified RNA. In one embodiment, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. Nos. 8,278,036, 8,691,966, and 8,835,108, each of which is incorporated by reference herein in its entirety.

In some embodiments, nucleoside-modified mRNA does not activate any pathophysiologic pathways, translates very efficiently and almost immediately following delivery, and serve as templates for continuous protein production in vivo lasting for several days to weeks (Karikó et al., 2008, Mol Ther 16:1833-1840; Karikó et al., 2012, Mol Ther 20:948-953). The amount of mRNA required to exert a physiological effect is small, making it applicable for human therapy. For example, as described herein, nucleoside-modified mRNA encoding a growth factor (e.g., PDGF-BB and BMP-2) has demonstrated the ability to induce periodontal tissue and bone regeneration. For example, in some instances, a growth factor encoded by nucleoside-modified mRNA induces greater periodontal tissue and/or bone regeneration or a greater reduction in gingival inflammation as compared to a growth factor encoded by non-modified mRNA.

In some instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In some embodiments, using mRNA rather than the protein also has many advantages. Half-lives of proteins in the circulation or in tissues are often short, thus protein treatment would need frequent dosing, while mRNA provides a template for continuous protein production for several days to weeks. Purification of proteins is problematic and they can contain aggregates and other impurities that cause adverse effects (Kromminga and Schellekens, 2005, Ann NY Acad Sci 1050:257-265).

In some embodiments, the nucleoside-modified RNA comprises the naturally occurring modified-nucleoside pseudouridine. In some embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Karikó et al., 2011, Nucleic Acids Research 39:e142; Karikó et al., 2012, Mol Ther 20:948-953; Karikó et al., 2005, Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892).

Similar effects as described for pseudouridine have also been observed for RNA containing 1-methyl-pseudouridine.

In some embodiments, the nucleoside-modified nucleic acid molecule is a purified nucleoside-modified nucleic acid molecule. For example, in some embodiments, the composition is purified to remove double-stranded contaminants. In some instances, a preparative high-performance liquid chromatography (HPLC) purification procedure is used to obtain pseudouridine-containing RNA that has superior translational potential and no innate immunogenicity (Karikó et al., 2011, Nucleic Acids Research 39:e142). Administering HPLC-purified, pseudouridine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy. In some embodiments, the nucleoside-modified nucleic acid molecule is purified using non-HPLC methods. In some instances, the nucleoside-modified nucleic acid molecule is purified using chromatography methods, including but not limited to HPLC and fast protein liquid chromatography (FPLC). An exemplary FPLC-based purification procedure is described in Weissman et al., 2013, Methods Mol Biol, 969: 43-54. Exemplary purification procedures are also described in U.S. Patent Application Publication No. US2016/0032316, which is hereby incorporated by reference in its entirety.

The present invention encompasses RNA, oligoribonucleotide, and polyribonucleotide molecules comprising pseudouridine or a modified nucleoside. In some embodiments, the composition comprises an isolated nucleic acid encoding an antigen, wherein the nucleic acid comprises a pseudouridine or a modified nucleoside. In some embodiments, the composition comprises a vector, comprising an isolated nucleic acid encoding an antigen, adjuvant, or combination thereof, wherein the nucleic acid comprises a pseudouridine or a modified nucleoside.

In one embodiment, the nucleoside-modified RNA of the invention is IVT RNA, as described elsewhere herein. For example, in some embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In one embodiment, the modified nucleoside is m¹acp³Ψ (1-methyl-3-(3-amino-3-carboxypropyl) pseudouridine. In another embodiment, the modified nucleoside is m¹Ψ (1-methylpseudouridine). In another embodiment, the modified nucleoside is Ψm (2′-O-methylpseudouridine). In another embodiment, the modified nucleoside is m⁵D (5-methyldihydrouridine). In another embodiment, the modified nucleoside is m³Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the nucleoside that is modified in the nucleoside-modified RNA the present invention is uridine (U). In another embodiment, the modified nucleoside is cytidine (C). In another embodiment, the modified nucleoside is adenosine (A). In another embodiment, the modified nucleoside is guanosine (G).

In another embodiment, the modified nucleoside of the present invention is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m′A (1-methyladenosine); m²A (2-methyladenosine); Am (2′-O-methyladenosine); ms² m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-dimethylguanosine); m²₂ Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵ s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In various embodiments, between 0.1% and 100% of the residues in the nucleoside-modified RNA of the present invention are modified (e.g., either by the presence of pseudouridine, 1-methyl-pseudouridine, or another modified nucleoside base). In one embodiment, the fraction of modified residues is 0.1%. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%. In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In some embodiments, the composition comprises a purified preparation of single-stranded nucleoside modified RNA. For example, in some embodiments, the purified preparation of single-stranded nucleoside modified RNA is substantially free of double stranded RNA (dsRNA). In some embodiments, the purified preparation is at least 90%, or at least 91%, or at least 92%, or at least 93% or at least 94%, or at least 95%, or at least 96%, or at least 97%, or at least 98%, or at least 99%, or at least 99.5%, or at least 99.9% single stranded nucleoside modified RNA, relative to all other nucleic acid molecules (DNA, dsRNA, etc.).

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 4-fold factor. In another embodiment, translation is enhanced by a 5-fold factor. In another embodiment, translation is enhanced by a 6-fold factor. In another embodiment, translation is enhanced by a 7-fold factor. In another embodiment, translation is enhanced by an 8-fold factor. In another embodiment, translation is enhanced by a 9-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In another embodiment, the nucleoside-modified antigen-encoding RNA of the present invention induces significantly more periodontal tissue regeneration as compared with an unmodified in vitro-synthesized RNA molecule of the same sequence. In another embodiment, the modified RNA molecule induces periodontal tissue regeneration that is 2-fold greater than its unmodified counterpart. In another embodiment, the periodontal tissue and/or bone regeneration is increased by a 3-fold factor. In another embodiment, the periodontal tissue and/or bone regeneration is increased by a 4-fold factor. In another embodiment, the periodontal tissue and/or bone regeneration is increased by a 5-fold factor. In another embodiment, the periodontal tissue and/or bone regeneration is increased by a 6-fold factor. In another embodiment, the periodontal tissue and/or bone regeneration is increased by a 7-fold factor. In another embodiment, the periodontal tissue and/or bone regeneration is increased by an 8-fold factor. In another embodiment, the periodontal tissue and/or bone regeneration is increased by a 9-fold factor. In another embodiment, the periodontal tissue and/or bone regeneration is increased by a 10-fold factor. In another embodiment, the periodontal tissue and/or bone regeneration is increased by a 15-fold factor. In another embodiment, the periodontal tissue and/or bone regeneration is increased by a 20-fold factor. In another embodiment, the periodontal tissue and/or bone regeneration is increased by a 50-fold factor. In another embodiment, the periodontal tissue and/or bone regeneration is increased by a 100-fold factor. In another embodiment, the periodontal tissue and/or bone regeneration is increased by a 200-fold factor. In another embodiment, periodontal tissue and/or bone regeneration is increased by a 500-fold factor. In another embodiment, the periodontal tissue and/or bone regeneration is increased by a 1000-fold factor. In another embodiment, the periodontal tissue and/or bone regeneration is increased by a 2000-fold factor. In another embodiment, the periodontal tissue and/or bone regeneration is increased by another fold difference.

In another embodiment, “induces significantly more periodontal tissue and/or bone regeneration” refers to a detectable increase in periodontal tissue and/or bone regeneration. In another embodiment, the term refers to a fold increase in the periodontal tissue and/or bone regeneration (e.g., 1 of the fold increases enumerated above). In another embodiment, the term refers to an increase such that the nucleoside-modified RNA can be administered at a lower dose or frequency than an unmodified RNA molecule while still inducing a similarly effective periodontal tissue and/or bone regeneration. In another embodiment, the increase is such that the nucleoside-modified RNA can be administered using a single dose to induce effective periodontal tissue and/or bone regeneration.

In another embodiment, the nucleoside-modified RNA of the present invention exhibits significantly less innate immunogenicity than an unmodified in vitro-synthesized RNA molecule of the same sequence. In another embodiment, the modified RNA molecule exhibits an innate immune response that is 2-fold less than its unmodified counterpart. In another embodiment, innate immunogenicity is reduced by a 3-fold factor. In another embodiment, innate immunogenicity is reduced by a 4-fold factor. In another embodiment, innate immunogenicity is reduced by a 5-fold factor. In another embodiment, innate immunogenicity is reduced by a 6-fold factor. In another embodiment, innate immunogenicity is reduced by a 7-fold factor. In another embodiment, innate immunogenicity is reduced by a 8-fold factor. In another embodiment, innate immunogenicity is reduced by a 9-fold factor. In another embodiment, innate immunogenicity is reduced by a 10-fold factor. In another embodiment, innate immunogenicity is reduced by a 15-fold factor. In another embodiment, innate immunogenicity is reduced by a 20-fold factor. In another embodiment, innate immunogenicity is reduced by a 50-fold factor. In another embodiment, innate immunogenicity is reduced by a 100-fold factor. In another embodiment, innate immunogenicity is reduced by a 200-fold factor. In another embodiment, innate immunogenicity is reduced by a 500-fold factor. In another embodiment, innate immunogenicity is reduced by a 1000-fold factor. In another embodiment, innate immunogenicity is reduced by a 2000-fold factor. In another embodiment, innate immunogenicity is reduced by another fold difference.

In another embodiment, “exhibits significantly less innate immunogenicity” refers to a detectable decrease in innate immunogenicity. In another embodiment, the term refers to a fold decrease in innate immunogenicity (e.g., 1 of the fold decreases enumerated above). In another embodiment, the term refers to a decrease such that an effective amount of the nucleoside-modified RNA can be administered without triggering a detectable innate immune response. In another embodiment, the term refers to a decrease such that the nucleoside-modified RNA can be repeatedly administered without eliciting an innate immune response sufficient to detectably reduce production of the protein encoded by the modified RNA. In another embodiment, the decrease is such that the nucleoside-modified RNA can be repeatedly administered without eliciting an innate immune response sufficient to eliminate detectable production of the protein encoded by the modified RNA.

### Lipid Nanoparticle

In one embodiment, delivery of nucleoside-modified RNA comprises any suitable delivery method, including exemplary RNA transfection methods described elsewhere herein. In some embodiments, delivery of a nucleoside-modified RNA to a subject comprises mixing the nucleoside-modified RNA with a transfection reagent prior to the step of contacting. In another embodiment, a method of present invention further comprises administering nucleoside-modified RNA together with the transfection reagent. In another embodiment, the transfection reagent is a cationic lipid reagent. In another embodiment, the transfection reagent is a cationic polymer reagent.

In another embodiment, the transfection reagent is a lipid-based transfection reagent. In another embodiment, the transfection reagent is a protein-based transfection reagent. In another embodiment, the transfection reagent is a carbohydrate-based transfection reagent. In another embodiment, the transfection reagent is a cationic lipid-based transfection reagent. In another embodiment, the transfection reagent is a cationic polymer-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity. In another embodiment, liposomes are hollow spherical vesicles composed of lipids arranged in a similar fashion as those lipids, which make up the cell membrane. They have, in another embodiment, an internal aqueous space for entrapping water-soluble compounds and range in size from 0.05 to several microns in diameter. In another embodiment, liposomes can deliver RNA to cells in a biologically active form.

In one embodiment, the composition comprises a lipid nanoparticle (LNP) and one or more nucleic acid molecules described herein. For example, in one embodiment, the composition comprises an LNP and one or more nucleoside-modified RNA molecules encoding one or more growth factors, or fragments or variants thereof.

The term “lipid nanoparticle” refers to a particle having at least one dimension on the order of nanometers (e.g., 1-1,000 nm), which includes one or more lipids, for example a lipid of Formula (I), (II) or (III). In some embodiments, lipid nanoparticles are included in a formulation comprising a nucleoside-modified RNA as described herein. In some embodiments, such lipid nanoparticles comprise a cationic lipid (e.g., a lipid of Formula (I), (II) or (III)) and one or more excipient selected from neutral lipids, charged lipids, steroids and polymer conjugated lipids (e.g., a pegylated lipid such as a pegylated lipid of structure (IV), such as compound Iva). In some embodiments, the nucleoside-modified RNA is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In some embodiments, the nucleoside-modified RNA, when present in the lipid nanoparticles, is resistant in aqueous solution to degradation with a nuclease.

The LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In one embodiment, the LNP comprises one or more cationic lipids, and one or more stabilizing lipids. Stabilizing lipids include neutral lipids and pegylated lipids.

In one embodiment, the LNP comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In some embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

Suitable amino lipids include those having the formula:

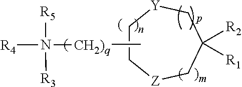

wherein R₁ and R₂ are either the same or different and independently optionally substituted C₁₀-C₂₄ alkyl, optionally substituted C₁₀-C₂₄ alkenyl, optionally substituted C₁₀-C₂₄ alkynyl, or optionally substituted C₁₀-C₂₄ acyl;

R₃ and R₄ are either the same or different and independently optionally substituted C₁-C₆ alkyl, optionally substituted C₂-C₆ alkenyl, or optionally substituted C₂-C₆ alkynyl or R₃ and R₄ may join to form an optionally substituted heterocyclic ring of 4 to 6 carbon atoms and 1 or 2 heteroatoms chosen from nitrogen and oxygen;

R₅ is either absent or present and when present is hydrogen or C₁-C₆ alkyl;

m, n, and p are either the same or different and independently either 0 or 1 with the proviso that m, n, and p are not simultaneously 0;

q is 0, 1, 2, 3, or 4; and

Y and Z are either the same or different and independently O, S, or NH.

In one embodiment, R₁ and R₂ are each linoleyl, and the amino lipid is a dilinoleyl amino lipid. In one embodiment, the amino lipid is a dilinoleyl amino lipid.

A representative useful dilinoleyl amino lipid has the formula:

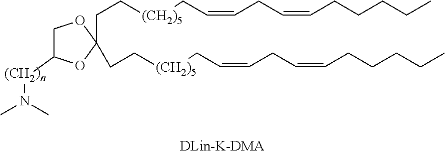

wherein n is 0, 1, 2, 3, or 4.

In one embodiment, the cationic lipid is a DLin-K-DMA. In one embodiment, the cationic lipid is DLin-KC2-DMA (DLin-K-DMA above, wherein n is 2).

In one embodiment, the cationic lipid component of the LNPs has the structure of Formula (I):

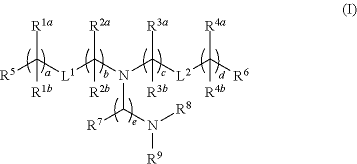

or a pharmaceutically acceptable salt, tautomer, prodrug or stereoisomer thereof, wherein:

L¹ and L² are each independently —O(C═O)—, —(C═O)O— or a carbon-carbon double bond;

R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;

R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;

R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;

R⁵ and R⁶ are each independently methyl or cycloalkyl;

R⁷ is, at each occurrence, independently H or C₁-C₁₂ alkyl;

R⁸ and R⁹ are each independently C₁-C₁₂ alkyl; or R⁸ and R⁹, together with the nitrogen atom to which they are attached, form a 5, 6 or 7-membered heterocyclic ring comprising one nitrogen atom;

a and d are each independently an integer from 0 to 24;

b and c are each independently an integer from 1 to 24; and

e is 1 or 2.

In some embodiments of Formula (I), at least one of R^{1a}, R^{2a}, R^{3a} or R^{4a} is C₁-C₁₂ alkyl, or at least one of L¹ or L² is —O(C═O)— or —(C═O)O—. In other embodiments, R^{1a} and R^{1b} are not isopropyl when a is 6 or n-butyl when a is 8.

In still further embodiments of Formula (I), at least one of R^{1a}, R^{2a}, R^{3a} or R^{4a} is C₁-C₁₂ alkyl, or at least one of L¹ or L² is —O(C═O)— or —(C═O)O—; and

R^{1a} and R^{1b} are not isopropyl when a is 6 or n-butyl when a is 8.

In other embodiments of Formula (I), R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;

In some embodiments of Formula (I), any one of L¹ or L² may be —O(C═O)— or a carbon-carbon double bond. L¹ and L² may each be —O(C═O)— or may each be a carbon-carbon double bond.

In some embodiments of Formula (I), one of L¹ or L² is —O(C═O)—. In other embodiments, both L¹ and L² are —O(C═O)—.

In some embodiments of Formula (I), one of L¹ or L² is —(C═O)O—. In other embodiments, both L¹ and L² are —(C═O)O—.

In some other embodiments of Formula (I), one of L¹ or L² is a carbon-carbon double bond. In other embodiments, both L¹ and L² are a carbon-carbon double bond.

In still other embodiments of Formula (I), one of L¹ or L² is —O(C═O)— and the other of L¹ or L² is —(C═O)O—. In more embodiments, one of L¹ or L² is —O(C═O)— and the other of L¹ or L² is a carbon-carbon double bond. In yet more embodiments, one of L¹ or L² is —(C═O)O— and the other of L¹ or L² is a carbon-carbon double bond.

It is understood that “carbon-carbon” double bond, as used throughout the specification, refers to one of the following structures:

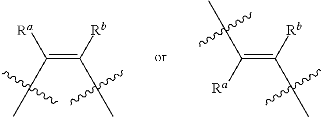

wherein R^{a} and R^{b} are, at each occurrence, independently H or a substituent. For example, in some embodiments R^{a} and R^{b} are, at each occurrence, independently H, C₁-C₁₂ alkyl or cycloalkyl, for example H or C₁-C₁₂ alkyl.

In other embodiments, the lipid compounds of Formula (I) have the following structure (Ia):

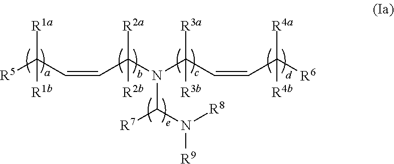

In other embodiments, the lipid compounds of Formula (I) have the following structure (Ib):

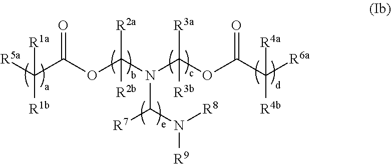

In yet other embodiments, the lipid compounds of Formula (I) have the following structure (Ic):

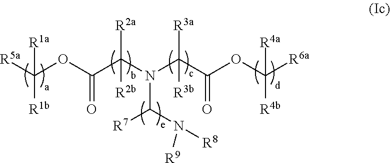

In some embodiments of the lipid compound of Formula (I), a, b, c and d are each independently an integer from 2 to 12 or an integer from 4 to 12. In other embodiments, a, b, c and d are each independently an integer from 8 to 12 or 5 to 9. In some embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some other embodiments of Formula (I), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some more embodiments of Formula (I), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some other embodiments of Formula (I), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some other various embodiments of Formula (I), a and d are the same. In some other embodiments, b and c are the same. In some other specific embodiments, a and d are the same and b and c are the same.

The sum of a and b and the sum of c and d in Formula (I) are factors which may be varied to obtain a lipid of Formula (I) having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such the sum of a and b and the sum of c and d is 12 or greater.

In some embodiments of Formula (I), e is 1. In other embodiments, e is 2.

The substituents at R^{1a}, R^{2a}, R^{3a} and R^{4a} of Formula (I) are not particularly limited. In some embodiments R^{1a}, R^{2a}, R^{3a} and R^{4a} are H at each occurrence. In some other embodiments at least one of R^{1a}, R^{2a}, R^{3a} and R^{4a} is C₁-C₁₂ alkyl. In some other embodiments at least one of R^{1a}, R^{2a}, R^{3a} and R^{4a} is C₁-C₈ alkyl. In some other embodiments at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In some embodiments of Formula (I), R^{1a}, R^{1b}, R^{4a} and R^{4b} are C₁-C₁₂ alkyl at each occurrence.

In further embodiments of Formula (I), at least one of R^{1b}, R^{2b}, R^{3b} and R^{4b} is H or R^{1b}, R^{2b}, R^{3b} and R^{4b} are H at each occurrence.

In some embodiments of Formula (I), R^{1b} together with the carbon atom to which it is bound is taken together with an adjacent R^{1b} and the carbon atom to which it is bound to form a carbon-carbon double bond. In other embodiments of the foregoing Rob together with the carbon atom to which it is bound is taken together with an adjacent Rob and the carbon atom to which it is bound to form a carbon-carbon double bond.

The substituents at R⁵ and R⁶ of Formula (I) are not particularly limited in the foregoing embodiments. In some embodiments one or both of R⁵ or R⁶ is methyl. In some other embodiments one or both of R⁵ or R⁶ is cycloalkyl for example cyclohexyl. In these embodiments the cycloalkyl may be substituted or not substituted. In some other embodiments the cycloalkyl is substituted with C₁-C₁₂ alkyl, for example tert-butyl.

The substituents at R⁷ are not particularly limited in the foregoing embodiments of Formula (I). In some embodiments at least one R⁷ is H. In some other embodiments, R⁷ is H at each occurrence. In some other embodiments R⁷ is C₁-C₁₂ alkyl.

In some other of the foregoing embodiments of Formula (I), one of R⁸ or R⁹ is methyl. In other embodiments, both R⁸ and R⁹ are methyl.

In some different embodiments of Formula (I), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring.

In various different embodiments, the lipid of Formula (I) has one of the structures set forth in Table 1 below.

TABLE 1
Representative Lipids of Formula (I)

| | | Prep. |
|---|---|---|
| No. | Structure | Method |
| I-1 | 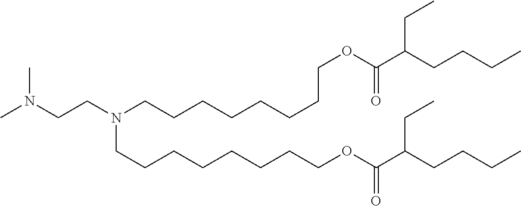 [SMILES: CCCCC(CC)C(=O)OCCCCCCCCN(CCCCCCCCOC(=O)C(CC)CCCC)CCN(C)C] | B |
| I-2 | 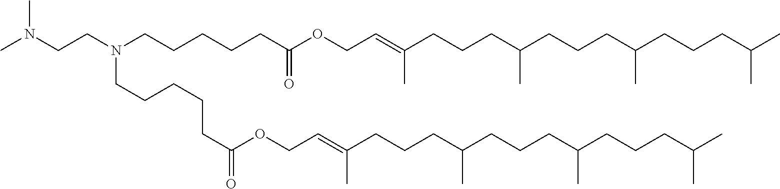 [SMILES: C/C(=C\COC(=O)CCCCCN(CCCCCC(=O)OC/C=C(\C)CCCC(C)CCCC(C)CCCC(C)C)CCN(C)C)CCCC(C)CCCC(C)CCCC(C)C] | A |
| I-3 | 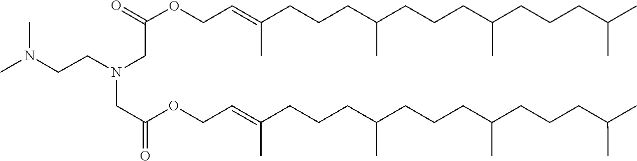 [SMILES: C/C(=C\COC(=O)CN(CCN(C)C)CC(=O)OC/C=C(\C)CCCC(C)CCCC(C)CCCC(C)C)CCCC(C)CCCC(C)CCCC(C)C] | A |
| I-4 | 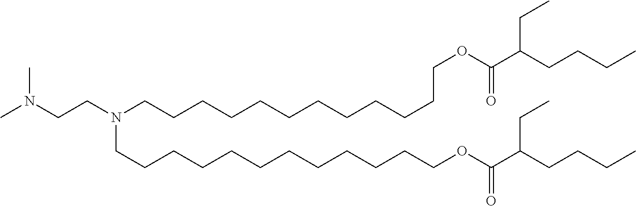 [SMILES: CCCCC(CC)C(=O)OCCCCCCCCCCCCN(CCCCCCCCCCCCOC(=O)C(CC)CCCC)CCN(C)C] | B |
| I-5 | 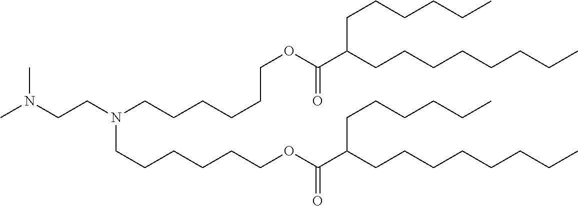 [SMILES: CCCCCCCCC(CCCCCC)C(=O)OCCCCCCN(CCCCCCOC(=O)C(CCCCCC)CCCCCCCC)CCN(C)C] | B |
| I-6 | 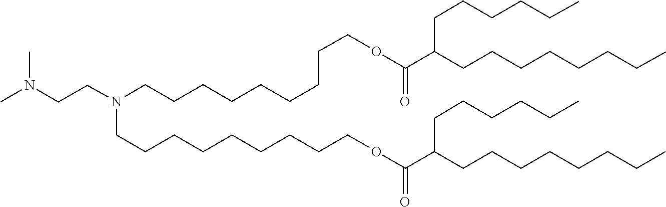 [SMILES: CCCCCCCCC(CCCCCC)C(=O)OCCCCCCCCCN(CCCCCCCCCOC(=O)C(CCCCCC)CCCCCCCC)CCN(C)C] | B |
| I-7 | 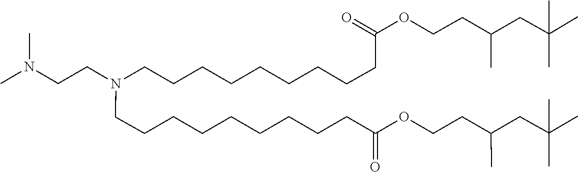 [SMILES: CC(CCOC(=O)CCCCCCCCCN(CCCCCCCCCC(=O)OCCC(C)CC(C)(C)C)CCN(C)C)CC(C)(C)C] | A |
| I-8 | 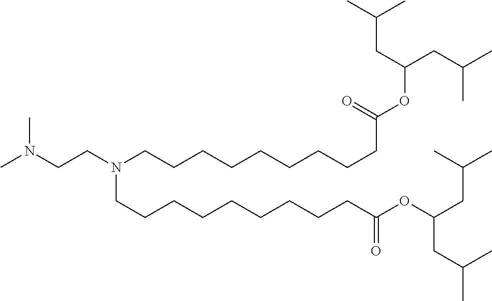 [SMILES: CC(C)CC(CC(C)C)OC(=O)CCCCCCCCCN(CCCCCCCCCC(=O)OC(CC(C)C)CC(C)C)CCN(C)C] | A |
| I-9 | 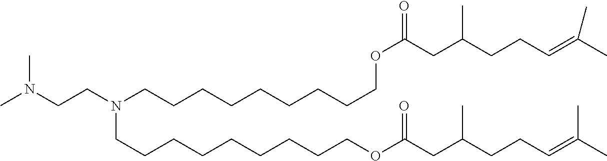 [SMILES: CC(C)=CCCC(C)CC(=O)OCCCCCCCCCN(CCCCCCCCCOC(=O)CC(C)CCC=C(C)C)CCN(C)C] | B |
| I-10 | 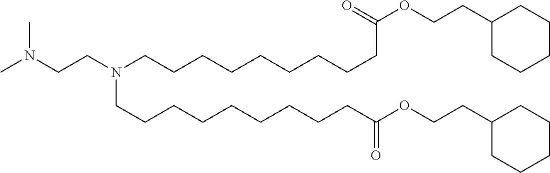 [SMILES: CN(C)CCN(CCCCCCCCCC(=O)OCCC1CCCCC1)CCCCCCCCCC(=O)OCCC1CCCCC1] | A |
| I-11 | 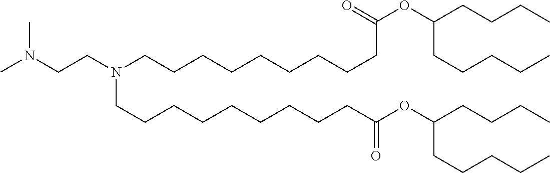 [SMILES: CCCCCC(CCCC)OC(=O)CCCCCCCCCN(CCCCCCCCCC(=O)OC(CCCC)CCCCC)CCN(C)C] | A |
| I-12 | 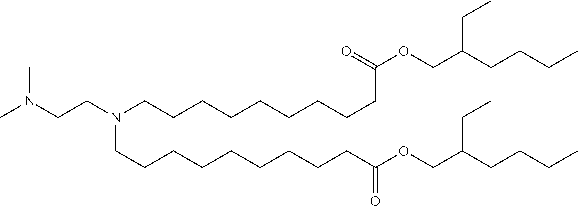 [SMILES: CCCCC(CC)COC(=O)CCCCCCCCCN(CCCCCCCCCC(=O)OCC(CC)CCCC)CCN(C)C] | A |
| I-13 | 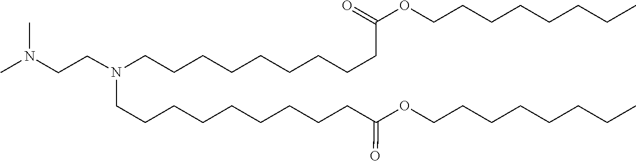 [SMILES: CCCCCCCCOC(=O)CCCCCCCCCN(CCCCCCCCCC(=O)OCCCCCCCC)CCN(C)C] | A |
| I-14 | 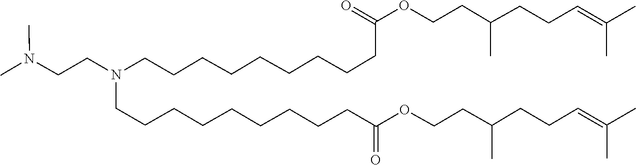 [SMILES: CC(C)=CCCC(C)CCOC(=O)CCCCCCCCCN(CCCCCCCCCC(=O)OCCC(C)CCC=C(C)C)CCN(C)C] | A |
| I-15 | 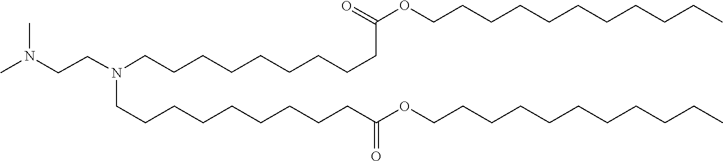 [SMILES: CCCCCCCCCCCOC(=O)CCCCCCCCCN(CCCCCCCCCC(=O)OCCCCCCCCCCC)CCN(C)C] | A |
| I-16 | 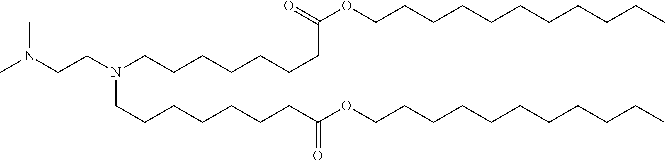 [SMILES: CCCCCCCCCCCOC(=O)CCCCCCCN(CCCCCCCC(=O)OCCCCCCCCCCC)CCN(C)C] | A |
| I-17 | 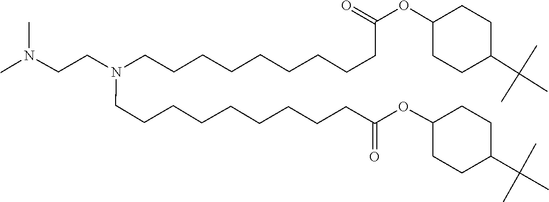 [SMILES: CN(C)CCN(CCCCCCCCCC(=O)OC1CCC(C(C)(C)C)CC1)CCCCCCCCCC(=O)OC1CCC(C(C)(C)C)CC1] | A |
| I-18 | 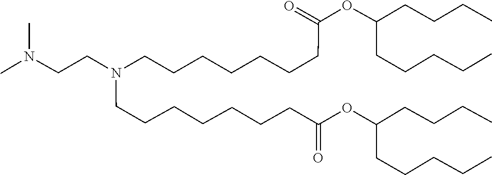 [SMILES: CCCCCC(CCCC)OC(=O)CCCCCCCN(CCCCCCCC(=O)OC(CCCC)CCCCC)CCN(C)C] | A |
| I-19 | 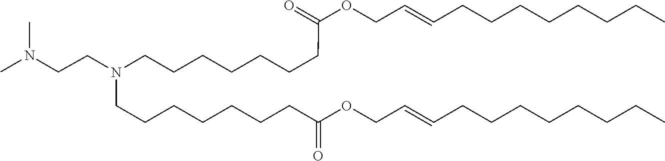 [SMILES: CCCCCCCC/C=C/COC(=O)CCCCCCCN(CCCCCCCC(=O)OC/C=C/CCCCCCCC)CCN(C)C] | A |
| I-20 | 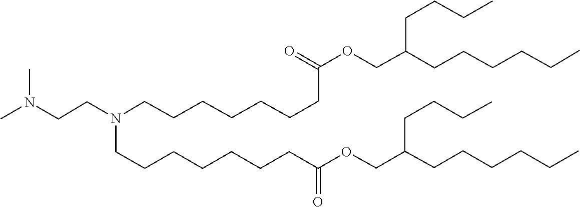 [SMILES: CCCCCCC(CCCC)COC(=O)CCCCCCCN(CCCCCCCC(=O)OCC(CCCC)CCCCCC)CCN(C)C] | A |
| I-21 | 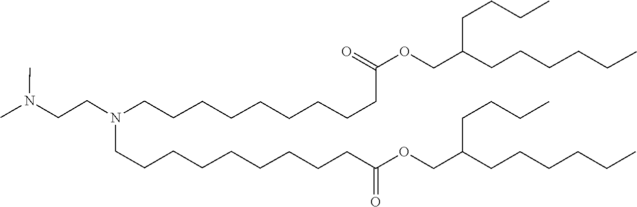 [SMILES: CCCCCCC(CCCC)COC(=O)CCCCCCCCCN(CCCCCCCCCC(=O)OCC(CCCC)CCCCCC)CCN(C)C] | A |
| I-22 | 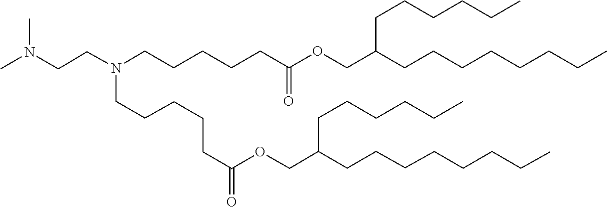 [SMILES: CCCCCCCCC(CCCCCC)COC(=O)CCCCCN(CCCCCC(=O)OCC(CCCCCC)CCCCCCCC)CCN(C)C] | A |
| I-23 | 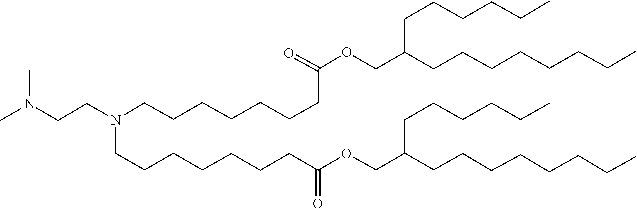 [SMILES: CCCCCCCCC(CCCCCC)COC(=O)CCCCCCCN(CCCCCCCC(=O)OCC(CCCCCC)CCCCCCCC)CCN(C)C] | A |
| I-24 | 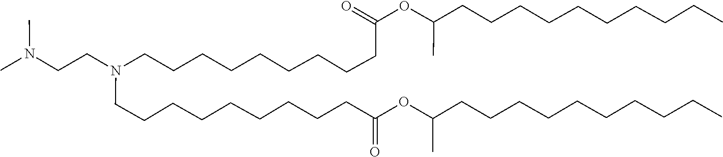 [SMILES: CCCCCCCCCCC(C)OC(=O)CCCCCCCCCN(CCCCCCCCCC(=O)OC(C)CCCCCCCCCC)CCN(C)C] | A |
| I-25 | 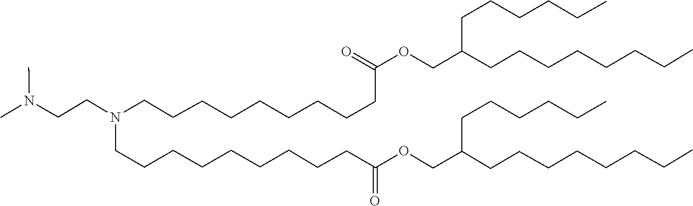 [SMILES: CCCCCCCCC(CCCCCC)COC(=O)CCCCCCCCCN(CCCCCCCCCC(=O)OCC(CCCCCC)CCCCCCCC)CCN(C)C] | A |
| I-26 | 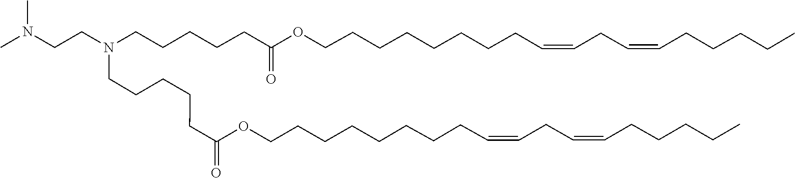 [SMILES: CCCCC/C=C\C/C=C\CCCCCCCCOC(=O)CCCCCN(CCCCCC(=O)OCCCCCCCC/C=C\C/C=C\CCCCC)CCN(C)C] | A |
| I-27 | 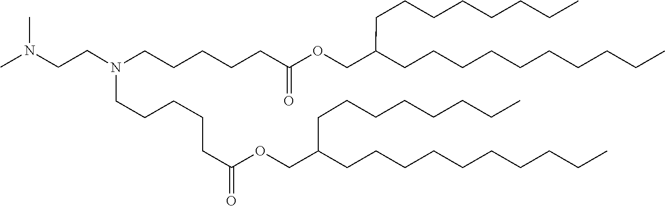 [SMILES: CCCCCCCCCCC(CCCCCCCC)COC(=O)CCCCCN(CCCCCC(=O)OCC(CCCCCCCC)CCCCCCCCCC)CCN(C)C] | A |
| I-28 | 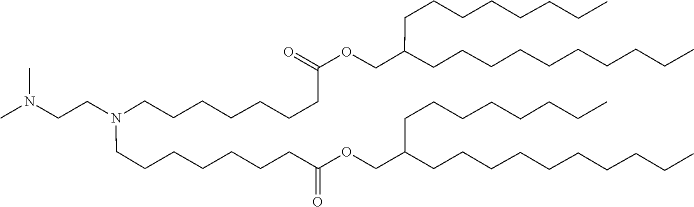 [SMILES: CCCCCCCCCCC(CCCCCCCC)COC(=O)CCCCCCCN(CCCCCCCC(=O)OCC(CCCCCCCC)CCCCCCCCCC)CCN(C)C] | A |
| I-29 | 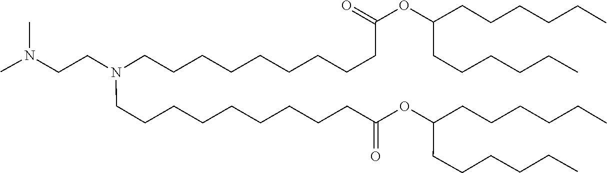 [SMILES: CCCCCCC(CCCCCC)OC(=O)CCCCCCCCCN(CCCCCCCCCC(=O)OC(CCCCCC)CCCCCC)CCN(C)C] | A |
| I-30 | 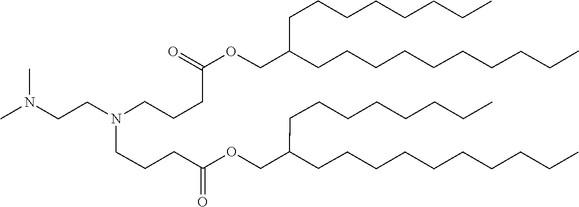 [SMILES: CCCCCCCCCCC(CCCCCCCC)COC(=O)CCCN(CCCC(=O)OCC(CCCCCCCC)CCCCCCCCCC)CCN(C)C] | A |
| I-31 | 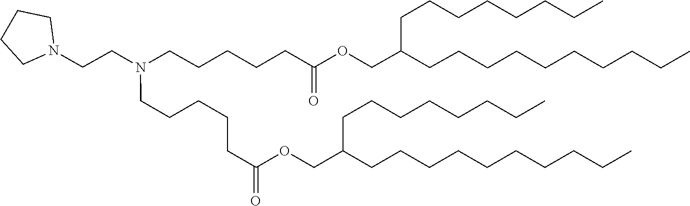 [SMILES: CCCCCCCCCCC(CCCCCCCC)COC(=O)CCCCCN(CCCCCC(=O)OCC(CCCCCCCC)CCCCCCCCCC)CCN1CCCC1] | C |
| I-32 | 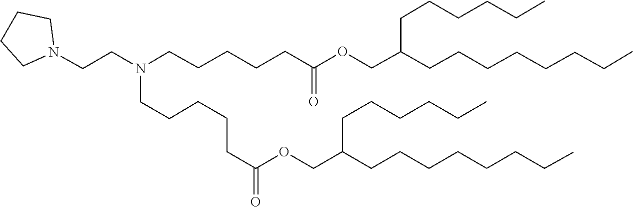 [SMILES: CCCCCCCCC(CCCCCC)COC(=O)CCCCCN(CCCCCC(=O)OCC(CCCCCC)CCCCCCCC)CCN1CCCC1] | C |
| I-33 | 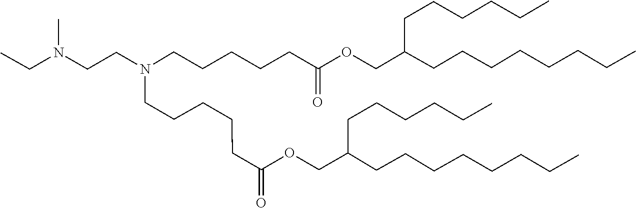 [SMILES: CCCCCCCCC(CCCCCC)COC(=O)CCCCCN(CCCCCC(=O)OCC(CCCCCC)CCCCCCCC)CCN(C)CC] | C |
| I-34 | 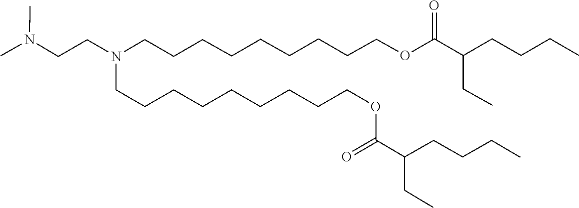 [SMILES: CCCCC(CC)C(=O)OCCCCCCCCCN(CCCCCCCCCOC(=O)C(CC)CCCC)CCN(C)C] | B |
| I-35 | 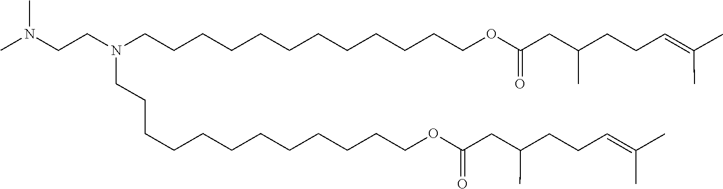 [SMILES: CC(C)=CCCC(C)CC(=O)OCCCCCCCCCCCCN(CCCCCCCCCCCCOC(=O)CC(C)CCC=C(C)C)CCN(C)C] | B |
| I-36 | 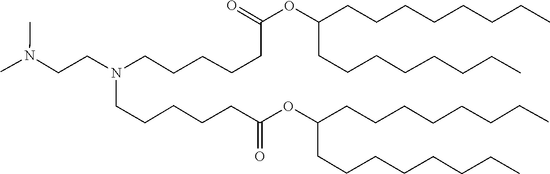 [SMILES: CCCCCCCCC(CCCCCCCC)OC(=O)CCCCCN(CCCCCC(=O)OC(CCCCCCCC)CCCCCCCC)CCN(C)C] | C |
| I-37 | 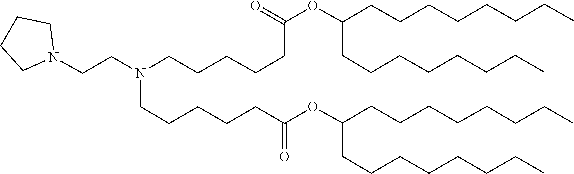 [SMILES: CCCCCCCCC(CCCCCCCC)OC(=O)CCCCCN(CCCCCC(=O)OC(CCCCCCCC)CCCCCCCC)CCN1CCCC1] | C |
| I-38 | 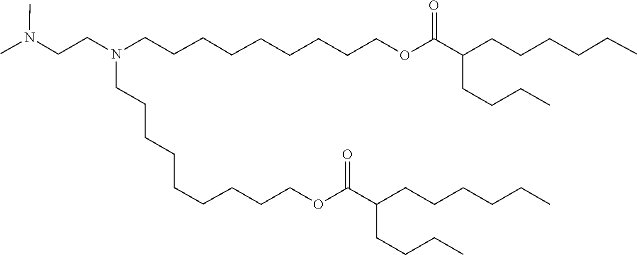 [SMILES: CCCCCCC(CCCC)C(=O)OCCCCCCCCCN(CCCCCCCCCOC(=O)C(CCCC)CCCCCC)CCN(C)C] | B |
| I-39 | 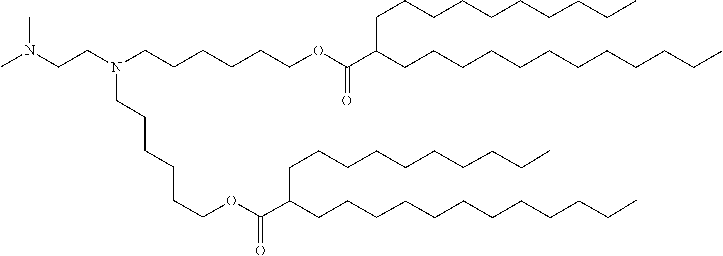 [SMILES: CCCCCCCCCCCCC(CCCCCCCCCC)C(=O)OCCCCCCN(CCCCCCOC(=O)C(CCCCCCCCCC)CCCCCCCCCCCC)CCN(C)C] | B |
| I-40 | 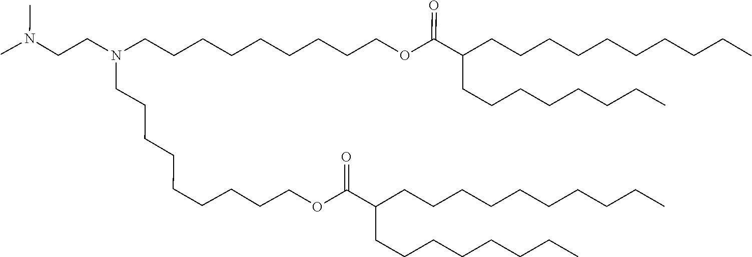 [SMILES: CCCCCCCCCCC(CCCCCCCC)C(=O)OCCCCCCCCCN(CCCCCCCCCOC(=O)C(CCCCCCCC)CCCCCCCCCC)CCN(C)C] | B |
| I-41 | 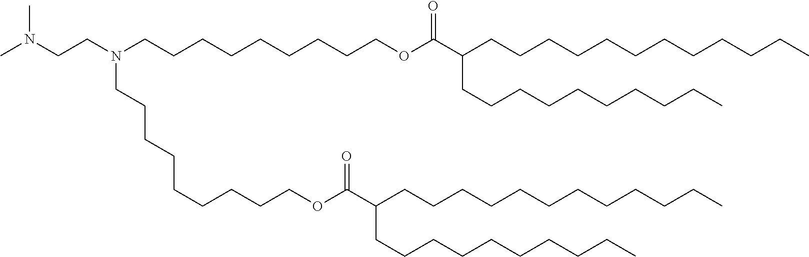 [SMILES: CCCCCCCCCCCCC(CCCCCCCCCC)C(=O)OCCCCCCCCCN(CCCCCCCCCOC(=O)C(CCCCCCCCCC)CCCCCCCCCCCC)CCN(C)C] | B |

In some embodiments, the LNPs comprise a lipid of Formula (I), a nucleoside-modified RNA and one or more excipients selected from neutral lipids, steroids and pegylated lipids. In some embodiments the lipid of Formula (I) is compound I-5. In some embodiments the lipid of Formula (I) is compound I-6.

In some other embodiments, the cationic lipid component of the LNPs has the structure of Formula (II):

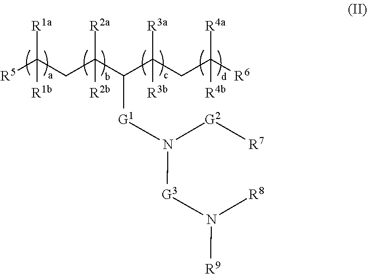

or a pharmaceutically acceptable salt, tautomer, prodrug or stereoisomer thereof, wherein:

L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}, —OC(═O)NR^{a}—, —NR^{a}C(═O)O—, or a direct bond;

G¹ is C₁-C₂ alkylene, —(C═O)—, —O(C═O)—, —SC(═O)—, —NR^{a}C(═O)— or a direct bond;

G² is —C(═O)—, —(C═O)O—, —C(═O)S—, —C(═O)NR^{a} or a direct bond;

G³ is C₁-C₆ alkylene;

R^{a} is H or C₁-C₁₂ alkyl;

R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;

R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;

R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;

R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;

R⁵ and R⁶ are each independently H or methyl;

R⁷ is C₄-C₂₀ alkyl;

R⁸ and R⁹ are each independently C₁-C₁₂ alkyl; or R⁸ and R⁹, together with the nitrogen atom to which they are attached, form a 5, 6 or 7-membered heterocyclic ring;

a, b, c and d are each independently an integer from 1 to 24; and

x is 0, 1 or 2.

In some embodiments of Formula (II), L¹ and L² are each independently —O(C═O)—, —(C═O)O— or a direct bond. In other embodiments, G¹ and G² are each independently —(C═O)— or a direct bond. In some different embodiments, L¹ and L² are each independently —O(C═O)—, —(C═O)O— or a direct bond; and G¹ and G² are each independently —(C═O)— or a direct bond.

In some different embodiments of Formula (II), L¹ and L² are each independently —C(═O)—, —O—, —S(O)ₓ—, —S—S—, —C(═O)S—, —SC(═O)—, —NR^{a}—, —NR^{a}C(═O)—, —C(═O)NR^{a}—, —NR^{a}C(═O)NR^{a}, —OC(═O)NR^{a}—, —NR^{a}C(═O)O—, —NR^{a}S(O)ₓNR^{a}—, —NR^{a}S(O)ₓ— or —S(O)ₓNR^{a}—.

In other of the foregoing embodiments of Formula (II), the lipid compound has one of the following structures (IIA) or (IIB):

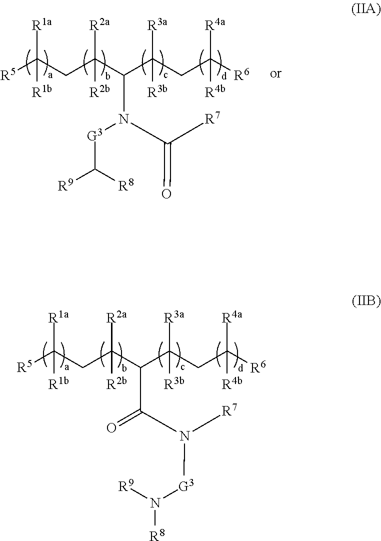

In some embodiments of Formula (II), the lipid compound has structure (IIA). In other embodiments, the lipid compound has structure (IIB).

In any of the foregoing embodiments of Formula (II), one of L¹ or L² is —O(C═O)—. For example, in some embodiments each of L¹ and L² are —O(C═O)—.

In some different embodiments of Formula (II), one of L¹ or L² is —(C═O)O—. For example, in some embodiments each of L¹ and L² is —(C═O)O—.

In different embodiments of Formula (II), one of L¹ or L² is a direct bond. As used herein, a “direct bond” means the group (e.g., L¹ or L²) is absent. For example, in some embodiments each of L¹ and L² is a direct bond.

In other different embodiments of Formula (II), for at least one occurrence of R^{1a} and R^{1b}, R^{1a} is H or C₁-C₁₂ alkyl, and R^{1b} together with the carbon atom to which it is bound is taken together with an adjacent R^{1b} and the carbon atom to which it is bound to form a carbon-carbon double bond.

In still other different embodiments of Formula (II), for at least one occurrence of R^{4a} and R^{4b}, R^{4a} is H or C₁-C₁₂ alkyl, and R^{4b} together with the carbon atom to which it is bound is taken together with an adjacent R^{4b} and the carbon atom to which it is bound to form a carbon-carbon double bond.

In more embodiments of Formula (II), for at least one occurrence of R^{2a} and R^{2b}, R^{2a} is H or C₁-C₁₂ alkyl, and R^{2b} together with the carbon atom to which it is bound is taken together with an adjacent R^{2b} and the carbon atom to which it is bound to form a carbon-carbon double bond.

In other different embodiments of Formula (II), for at least one occurrence of R^{3a} and R^{3b}, R^{3a} is H or C₁-C₁₂ alkyl, and R^{3b} together with the carbon atom to which it is bound is taken together with an adjacent R^{3b} and the carbon atom to which it is bound to form a carbon-carbon double bond.

In various other embodiments of Formula (II), the lipid compound has one of the following structures (IIC) or (IID):

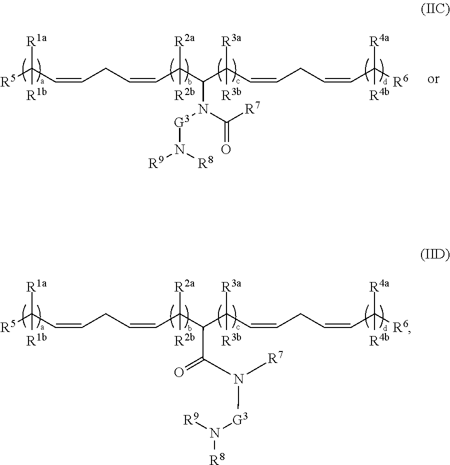

wherein e, f, g and h are each independently an integer from 1 to 12.

In some embodiments of Formula (II), the lipid compound has structure (IIC). In other embodiments, the lipid compound has structure (IID).

In various embodiments of structures (IIC) or (IID), e, f, g and h are each independently an integer from 4 to 10.

In some embodiments of Formula (II), a, b, c and d are each independently an integer from 2 to 12 or an integer from 4 to 12. In other embodiments, a, b, c and d are each independently an integer from 8 to 12 or 5 to 9. In some embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some embodiments of Formula (II), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some embodiments of Formula (II), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some embodiments of Formula (II), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some embodiments of Formula (II), e is 1. In other embodiments, e is 2. In more embodiments, e is 3. In yet other embodiments, e is 4. In some embodiments, e is 5. In other embodiments, e is 6. In more embodiments, e is 7. In yet other embodiments, e is 8. In some embodiments, e is 9. In other embodiments, e is 10. In more embodiments, e is 11. In yet other embodiments, e is 12.

In some embodiments of Formula (II), f is 1. In other embodiments, f is 2. In more embodiments, f is 3. In yet other embodiments, f is 4. In some embodiments, f is 5. In other embodiments, f is 6. In more embodiments, f is 7. In yet other embodiments, f is 8. In some embodiments, f is 9. In other embodiments, f is 10. In more embodiments, f is 11. In yet other embodiments, f is 12.

In some embodiments of Formula (II), g is 1. In other embodiments, g is 2. In more embodiments, g is 3. In yet other embodiments, g is 4. In some embodiments, g is 5. In other embodiments, g is 6. In more embodiments, g is 7. In yet other embodiments, g is 8. In some embodiments, g is 9. In other embodiments, g is 10. In more embodiments, g is 11. In yet other embodiments, g is 12.

In some embodiments of Formula (II), his 1. In other embodiments, e is 2. In more embodiments, h is 3. In yet other embodiments, h is 4. In some embodiments, e is 5. In other embodiments, h is 6. In more embodiments, h is 7. In yet other embodiments, h is 8. In some embodiments, h is 9. In other embodiments, h is 10. In more embodiments, his 11. In yet other embodiments, h is 12.

In some other various embodiments of Formula (II), a and d are the same. In some other embodiments, b and c are the same. In some other specific embodiments and a and d are the same and b and c are the same.

The sum of a and b and the sum of c and d of Formula (II) are factors which may be varied to obtain a lipid having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a. b, c and d are selected such that the sum of a and b and the sum of c and d is 12 or greater.

The substituents at R^{1a}, R^{2a}, R^{3a} and R^{4a} of Formula (II) are not particularly limited. In some embodiments, at least one of R^{1a}, R^{2a}, R^{3a} and R^{4a} is H. In some embodiments R^{1a}, R^{2a}, R^{3a} and R^{4a} are H at each occurrence. In some other embodiments at least one of R^{1a}, R^{2a}, R^{3a} and R^{4a} is C₁-C₁₂ alkyl. In some other embodiments at least one of R^{1a}, R^{2a}, R^{3a} and R^{4a} is C₁-C₈ alkyl. In some other embodiments at least one of R^{1a}, R^{2a}, R^{3a} and R^{4a} is C₁-C₆ alkyl. In some of the foregoing embodiments, the C₁-C₈ alkyl is methyl, ethyl, n-propyl, iso-propyl, n-butyl, iso-butyl, tert-butyl, n-hexyl or n-octyl.

In some embodiments of Formula (II), R^{1a}, R^{1b}, R^{4a} and R^{4b} are C₁-C₁₂ alkyl at each occurrence.

In further embodiments of Formula (II), at least one of R^{1b}, R^{2b}, R^{3b} and R^{4b} is H or R^{1b}, R^{2b}, R^{3b} and R^{4b} are H at each occurrence.

In some embodiments of Formula (II), R^{1b} together with the carbon atom to which it is bound is taken together with an adjacent R^{1b} and the carbon atom to which it is bound to form a carbon-carbon double bond. In other embodiments of the foregoing R^{4b} together with the carbon atom to which it is bound is taken together with an adjacent R^{4b} and the carbon atom to which it is bound to form a carbon-carbon double bond.

The substituents at R⁵ and R⁶ of Formula (II) are not particularly limited in the foregoing embodiments. In some embodiments one of R⁵ or R⁶ is methyl. In other embodiments each of R⁵ or R⁶ is methyl.

The substituents at R⁷ of Formula (II) are not particularly limited in the foregoing embodiments. In some embodiments R⁷ is C₆-C₁₆ alkyl. In some other embodiments, R⁷ is C₆-C₉ alkyl. In some of these embodiments, R⁷ is substituted with —(C═O)OR^{b}, —O(C═O)R^{b}, —C(═O)R^{b}, —OR^{b}, —S(O)ₓR^{b}, —S—SR^{b}, —C(═O)SR^{b}, —SC(═O)R^{b}, —NR^{a}R^{b}, —NR^{a}C(═O)R^{b}, —C(═O)NR^{a}R^{b}, —NR^{a}C(═O)NR^{a}R^{b}, —OC(═O)NR^{a}R^{b}, —NR^{a}C(═O)OR^{b}, —NR^{a}S(O)ₓNR^{a}R^{b}, —NR^{a}S(O)ₓR^{b} or —S(O)ₓNR^{a}R^{b}, wherein: R^{a} is H or C₁-C₁₂ alkyl; R^{b} is C₁-C₁₅ alkyl; and x is 0, 1 or 2. For example, in some embodiments R⁷ is substituted with —(C═O)OR^{b} or —O(C═O)R^{b}.

In various of the foregoing embodiments of Formula (II), R^{b} is branched C₁-C₁₅ alkyl. For example, in some embodiments R^{b} has one of the following structures:

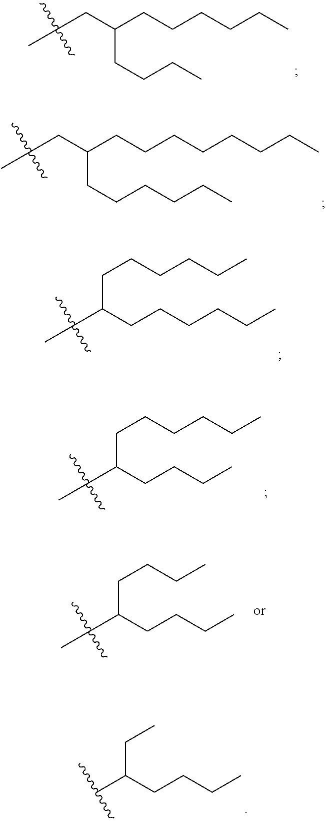

In some other of the foregoing embodiments of Formula (II), one of R⁸ or R⁹ is methyl. In other embodiments, both R⁸ and R⁹ are methyl.

In some different embodiments of Formula (II), R⁸ and R⁹, together with the nitrogen atom to which they are attached, form a 5, 6 or 7-membered heterocyclic ring. In some embodiments of the foregoing, R⁸ and R⁹, together with the nitrogen atom to which they are attached, form a 5-membered heterocyclic ring, for example a pyrrolidinyl ring. In some different embodiments of the foregoing, R⁸ and R⁹, together with the nitrogen atom to which they are attached, form a 6-membered heterocyclic ring, for example a piperazinyl ring.

In still other embodiments of the foregoing lipids of Formula (II), G³ is C₂-C₄ alkylene, for example C₃ alkylene.

In various different embodiments, the lipid compound has one of the structures set forth in Table 2 below.

TABLE 2
Representative Lipids of Formula (II)

| | | Prep. |
|---|---|---|
| No. | Structure | Method |
| II-1 | 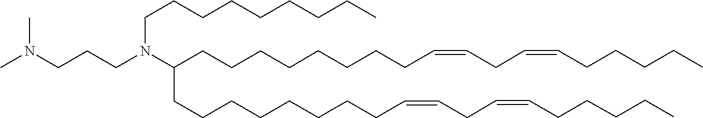 [SMILES: CCCCC/C=C\C/C=C\CCCCCCCCC(CCCCCCCC/C=C\C/C=C\CCCCC)N(CCCCCCCCC)CCCN(C)C] | D |
| II-2 | 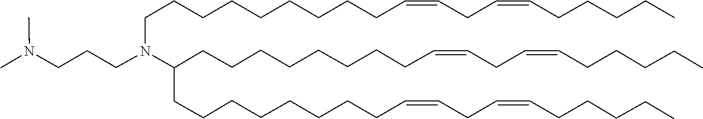 [SMILES: CCCCC/C=C\C/C=C\CCCCCCCCCN(CCCN(C)C)C(CCCCCCCC/C=C\C/C=C\CCCCC)CCCCCCCC/C=C\C/C=C\CCCCC] | D |
| II-3 | 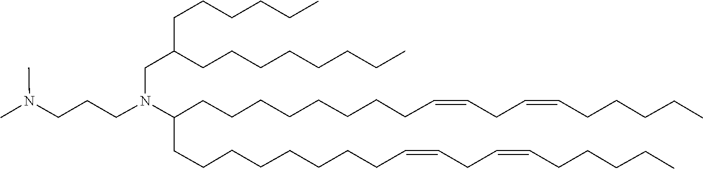 [SMILES: CCCCC/C=C\C/C=C\CCCCCCCCC(CCCCCCCC/C=C\C/C=C\CCCCC)N(CCCN(C)C)CC(CCCCCC)CCCCCCCC] | D |
| II-4 | 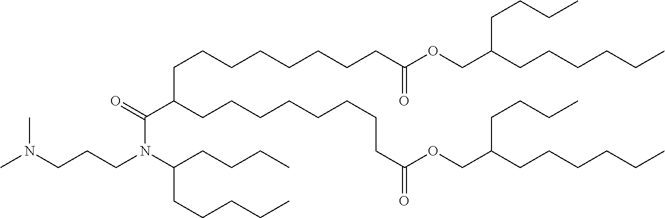 [SMILES: CCCCCCC(CCCC)COC(=O)CCCCCCCCC(CCCCCCCCC(=O)OCC(CCCC)CCCCCC)C(=O)N(CCCN(C)C)C(CCCC)CCCCC] | E |
| II-5 | 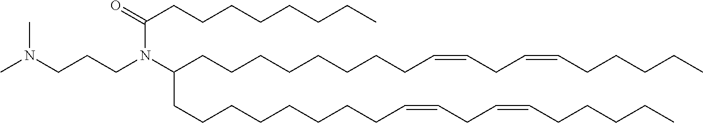 [SMILES: CCCCC/C=C\C/C=C\CCCCCCCCC(CCCCCCCC/C=C\C/C=C\CCCCC)N(CCCN(C)C)C(=O)CCCCCCCC] | D |
| II-6 | 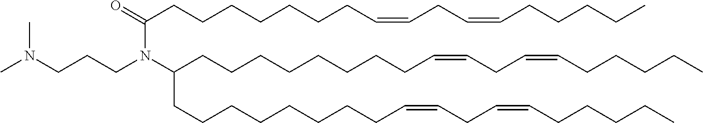 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC] | D |
| II-7 | 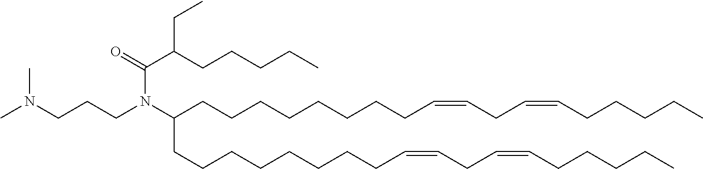 [SMILES: CCCCC/C=C\C/C=C\CCCCCCCCC(CCCCCCCC/C=C\C/C=C\CCCCC)N(CCCN(C)C)C(=O)C(CC)CCCCC] | D |
| II-8 | 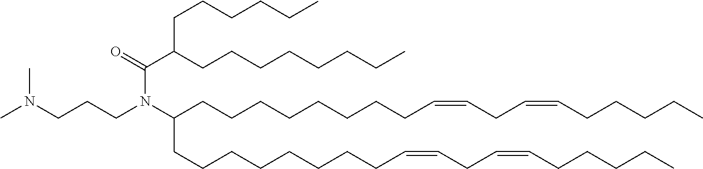 [SMILES: CCCCC/C=C\C/C=C\CCCCCCCCC(CCCCCCCC/C=C\C/C=C\CCCCC)N(CCCN(C)C)C(=O)C(CCCCCC)CCCCCCCC] | D |
| II-9 | 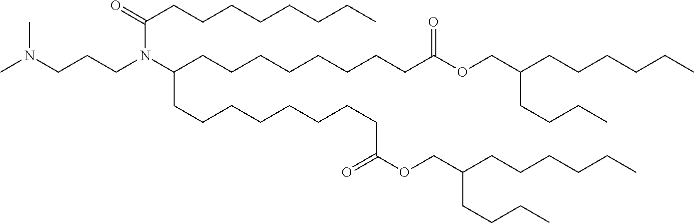 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] | D |
| II-10 | 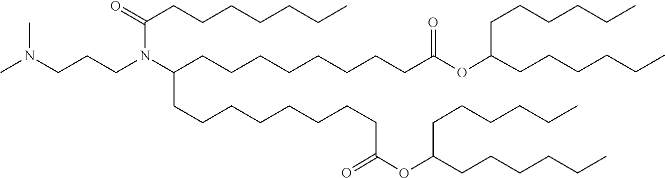 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-11 | 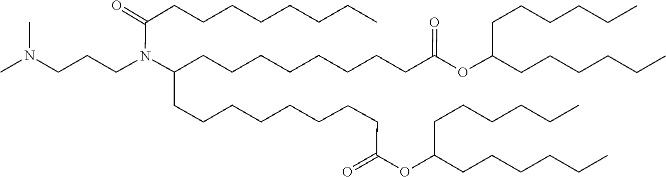 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-12 | 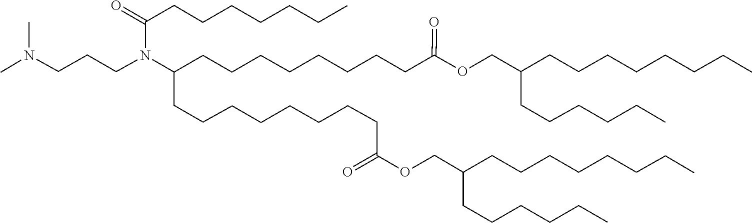 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] | D |
| II-13 | 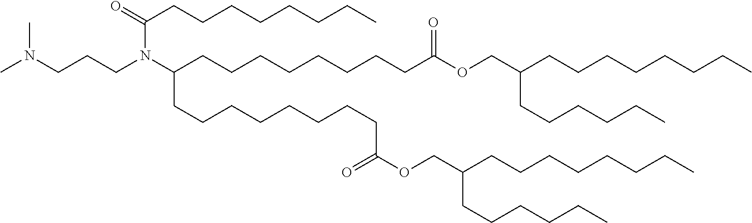 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] | D |
| II-14 | 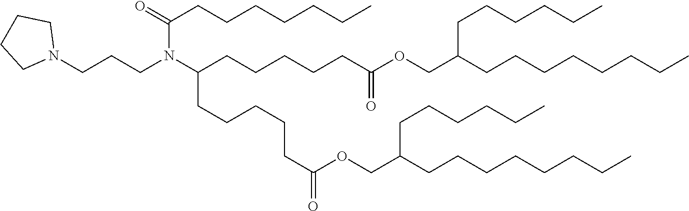 [SMILES: CCCCCCCCC(CCCCCC)COC(=O)CCCCCC(CCCCCC(=O)OCC(CCCCCC)CCCCCCCC)N(CCCN1CCCC1)C(=O)CCCCCCC] | D |
| II-15 | 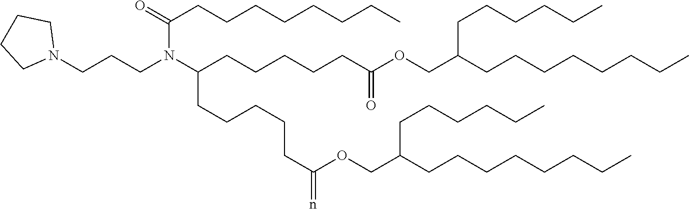 | D |
| 1I-16 | 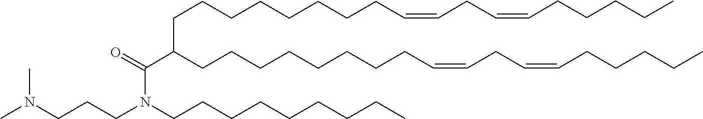 [SMILES: CCCCC/C=C\C/C=C\CCCCCCCCC(CCCCCCCC/C=C\C/C=C\CCCCC)C(=O)N(CCCCCCCCC)CCCN(C)C] | E |
| II-17 | 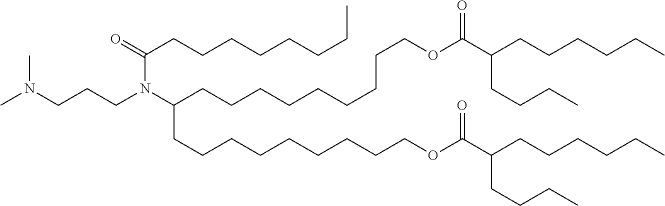 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC] | D |
| II-18 | 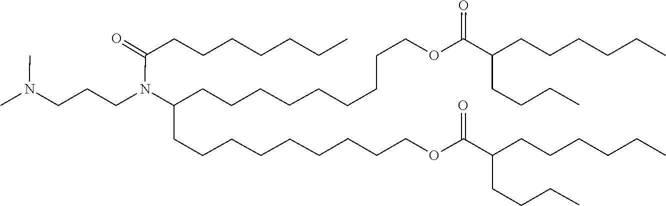 [SMILES: CCCCCCCC(=O)N(CCCN(C)C)C(CCCCCCCCCOC(=O)C(CCCC)CCCCCC)CCCCCCCCCOC(=O)C(CCCC)CCCCCC] | D |
| II-19 | 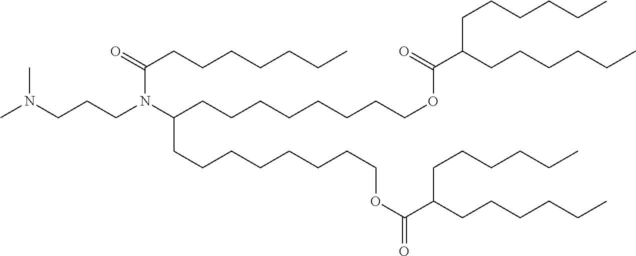 [SMILES: CCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-20 | 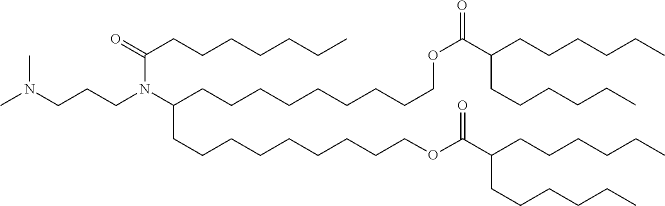 [SMILES: CCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-21 | 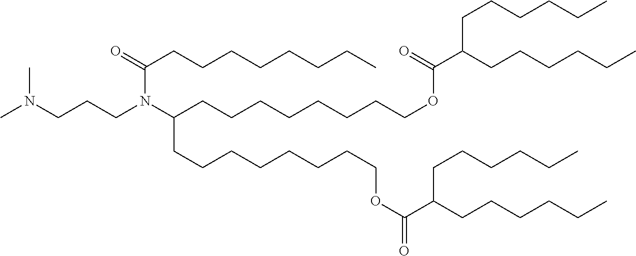 [SMILES: CCCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-22 | 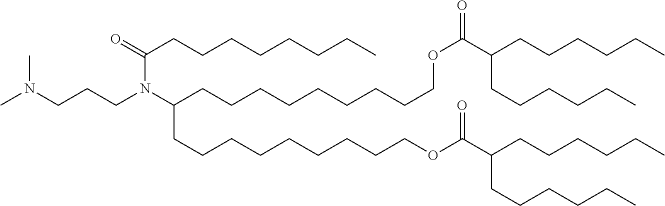 [SMILES: CCCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-23 | 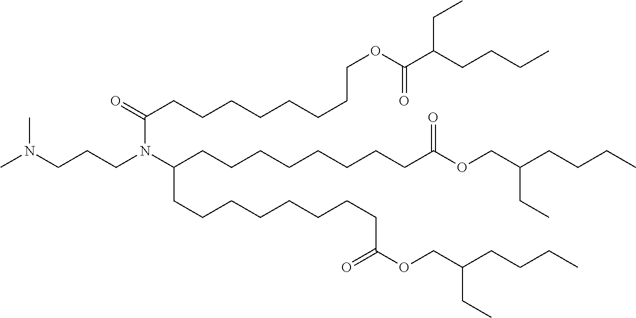 [SMILES: CCCCC(CC)COC(=O)CCCCCCCCC(CCCCCCCCC(=O)OCC(CC)CCCC)N(CCCN(C)C)C(=O)CCCCCCCCOC(=O)C(CC)CCCC] | D |
| II-24 | 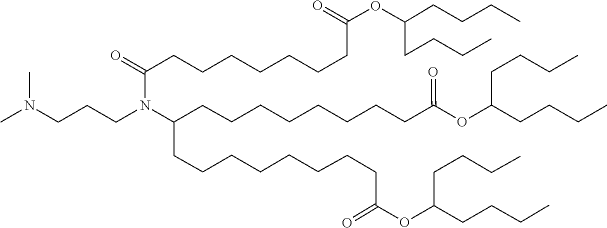 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] | D |
| II-25 | 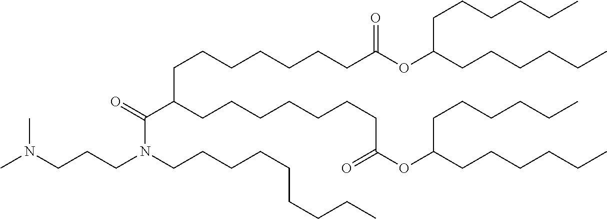 [SMILES: CCCCCCCCCN(CCCN(C)C)C(=O)C(CCCCCCCC(=O)OC(CCCCCC)CCCCCC)CCCCCCCC(=O)OC(CCCCCC)CCCCCC] | E |
| II-26 | 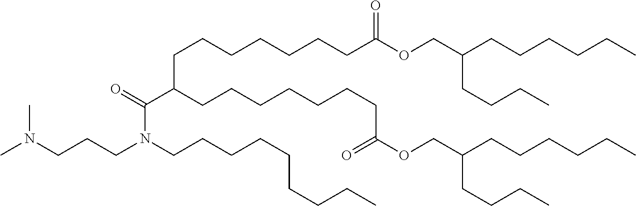 [SMILES: CCCCCCCCCN(CCCN(C)C)C(=O)C(CCCCCCCC(=O)OCC(CCCC)CCCCCC)CCCCCCCC(=O)OCC(CCCC)CCCCCC] | E |
| II-27 | 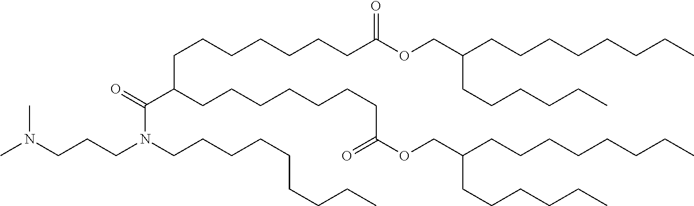 [SMILES: CCCCCCCCCN(CCCN(C)C)C(=O)C(CCCCCCCC(=O)OCC(CCCCCC)CCCCCCCC)CCCCCCCC(=O)OCC(CCCCCC)CCCCCCCC] | E |
| II-28 | 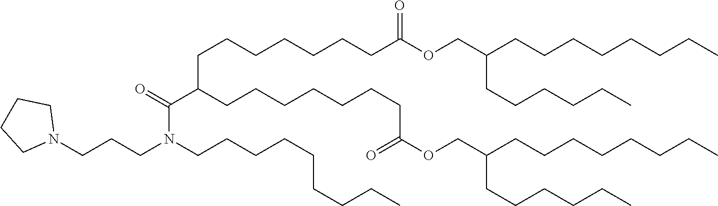 [SMILES: CCCCCCCCCN(CCCN1CCCC1)C(=O)C(CCCCCCCC(=O)OCC(CCCCCC)CCCCCCCC)CCCCCCCC(=O)OCC(CCCCCC)CCCCCCCC] | E |
| II-29 | 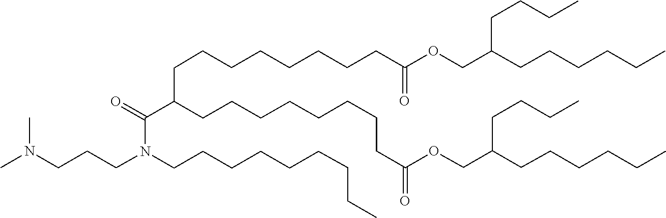 [SMILES: CCCCCCCCCN(CCCN(C)C)C(=O)C(CCCCCCCCC(=O)OCC(CCCC)CCCCCC)CCCCCCCCC(=O)OCC(CCCC)CCCCCC] | E |
| II-30 | 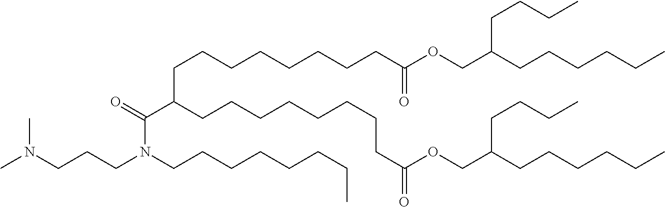 [SMILES: CCCCCCCCN(CCCN(C)C)C(=O)C(CCCCCCCCC(=O)OCC(CCCC)CCCCCC)CCCCCCCCC(=O)OCC(CCCC)CCCCCC] | E |
| II-31 | 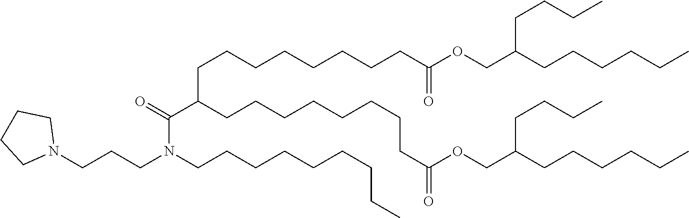 [SMILES: CCCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-32 | 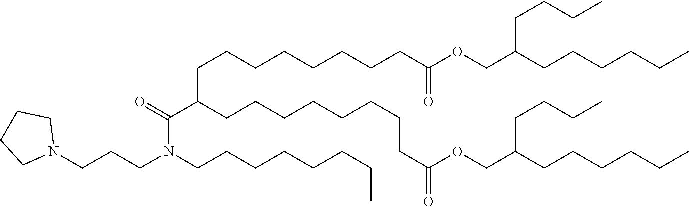 [SMILES: CCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-33 | 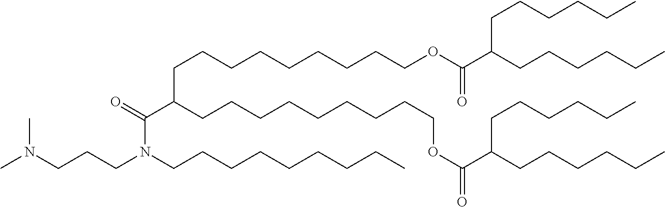 [SMILES: CCCCCCCCCN(CCCN(C)C)C(=O)C(CCCCCCCCCOC(=O)C(CCCCCC)CCCCCC)CCCCCCCCCOC(=O)C(CCCCCC)CCCCCC] | E |
| II-34 | 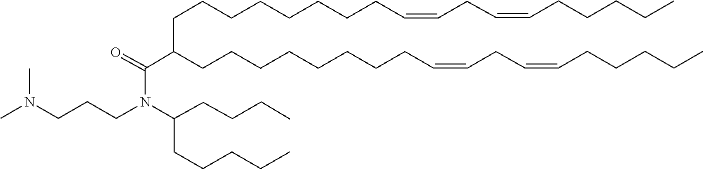 [SMILES: CCCCC/C=C\C/C=C\CCCCCCCCC(CCCCCCCC/C=C\C/C=C\CCCCC)C(=O)N(CCCN(C)C)C(CCCC)CCCCC] | E |
| II-35 | 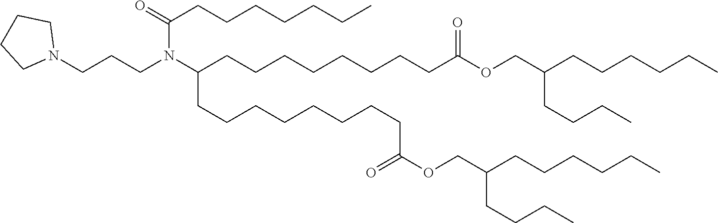 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC] | D |
| II-36 | 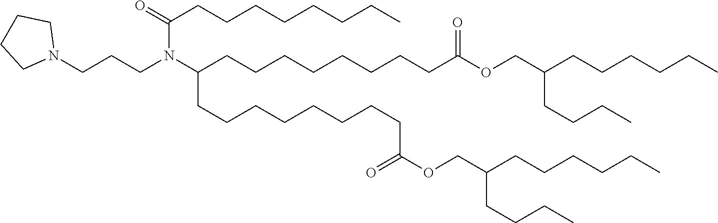 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC] | D |

In some embodiments, the LNPs comprise a lipid of Formula (II), a nucleoside-modified RNA and one or more excipient selected from neutral lipids, steroids and pegylated lipids. In some embodiments the lipid of Formula (II) is compound II-9. In some embodiments the lipid of Formula (II) is compound II-10. In some embodiments the lipid of Formula (II) is compound II-11. In some embodiments the lipid of Formula (II) is compound II-12. In some embodiments the lipid of Formula (II) is compound II-32.

In some other embodiments, the cationic lipid component of the LNPs has the structure of Formula (III):

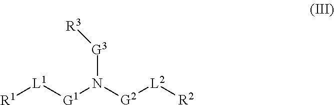

or a pharmaceutically acceptable salt, tautomer, prodrug or stereoisomer thereof,
wherein:

one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;

G¹ and G² are each independently unsubstituted C₁-C₁₂ alkylene or C₁-C₁₂ alkenylene;

G³ is C₁-C₂₄ alkylene, C₁-C₂₄ alkenylene, C₃-C₈ cycloalkylene, C₃-C₈ cycloalkenylene;

R^{a} is H or C₁-C₁₂ alkyl;

R¹ and R² are each independently C₆-C₂₄ alkyl or C₆-C₂₄ alkenyl;

R³ is H, OR⁵, CN, —C(═O)OR⁴, —OC(═O)R⁴ or —NR⁵C(═O)R⁴;

R⁴ is C₁-C₁₂ alkyl;

R⁵ is H or C₁-C₆ alkyl; and

x is 0, 1 or 2.

In some of the foregoing embodiments of Formula (III), the lipid has one of the following structures (IIIA) or (IIIB):

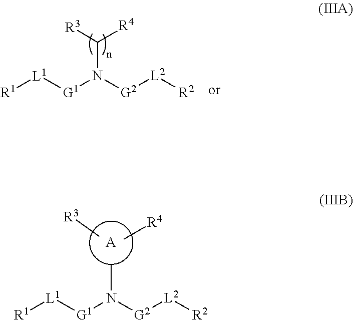

wherein:

A is a 3 to 8-membered cycloalkyl or cycloalkylene ring;

R⁶ is, at each occurrence, independently H, OH or C₁-C₂₄ alkyl;

n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

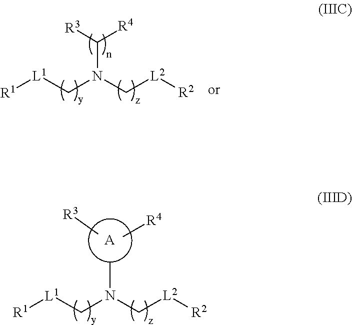

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

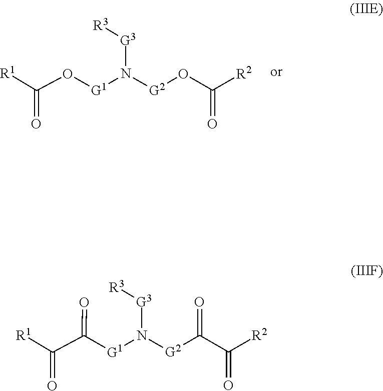

In some of the foregoing embodiments of Formula (III), the lipid has one of the following structures (IIIG), (IIIH), (IIII), or (IIIJ):

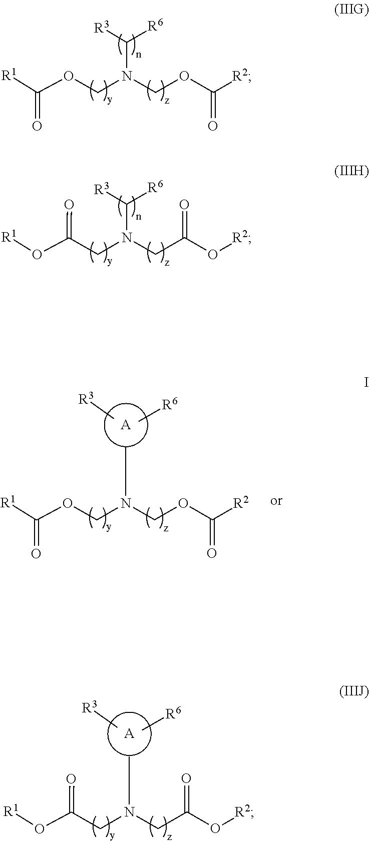

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G3 is substituted. In various different embodiments, G³ is linear C₁-C₂₄ alkylene or linear C₁-C₂₄ alkenylene.

In some other foregoing embodiments of Formula (III), le or R², or both, is C₆-C₂₄ alkenyl. For example, in some embodiments, R¹ and R² each, independently have the following structure:

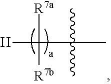

wherein:

R^{7a} and R^{7b} are, at each occurrence, independently H or C₁-C₁₂ alkyl; and

a is an integer from 2 to 12,

wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

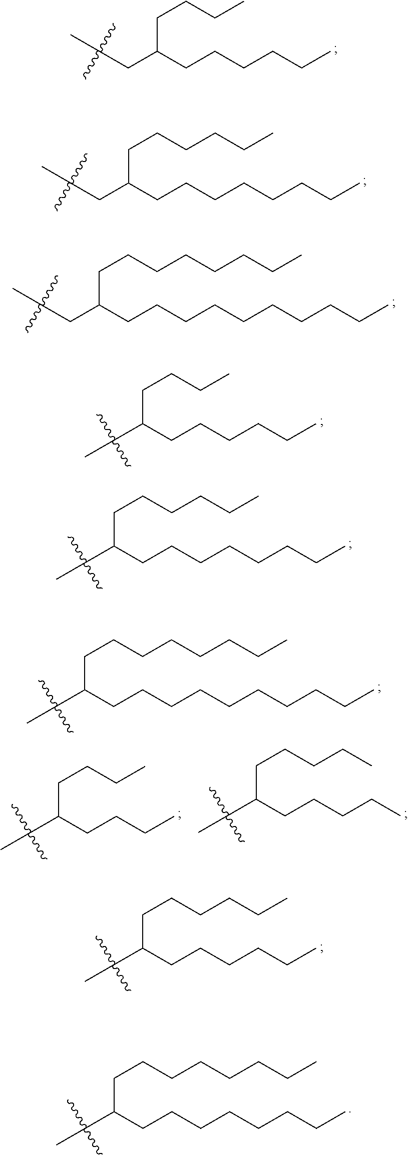

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in Table 3 below.

TABLE 3
Representative Compounds of Formula (III)

| | | Prep. |
|---|---|---|
| No. | Structure | Method |
| III-1 | 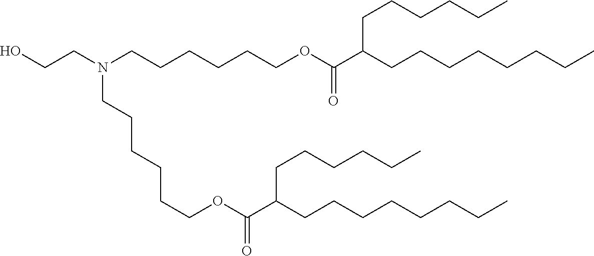 [SMILES: CCCCCCCCC(CCCCCC)C(=O)OCCCCCCN(CCO)CCCCCCOC(=O)C(CCCCCC)CCCCCCCC] | F |
| III-2 | 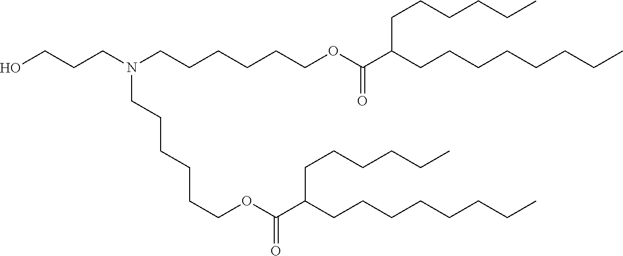 [SMILES: CCCCCCCCC(CCCCCC)C(=O)OCCCCCCN(CCCO)CCCCCCOC(=O)C(CCCCCC)CCCCCCCC] | F |
| III-3 | 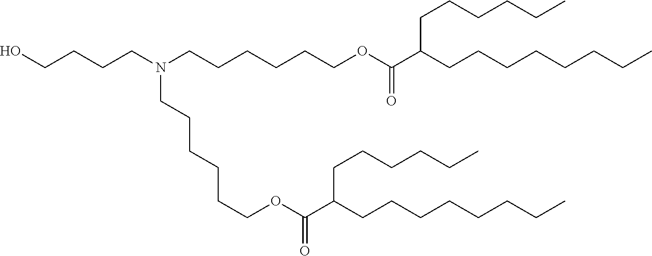 [SMILES: CCCCCCCCC(CCCCCC)C(=O)OCCCCCCN(CCCCO)CCCCCCOC(=O)C(CCCCCC)CCCCCCCC] | F |
| III-4 | 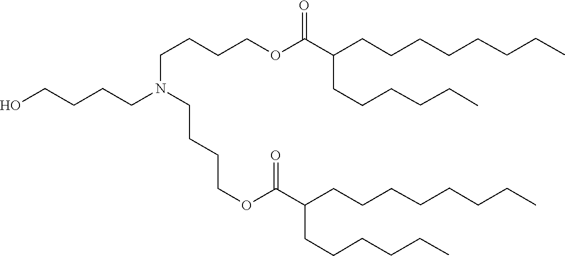 [SMILES: CCCCCCCCC(CCCCCC)C(=O)OCCCCN(CCCCO)CCCCOC(=O)C(CCCCCC)CCCCCCCC] | F |
| III-5 | 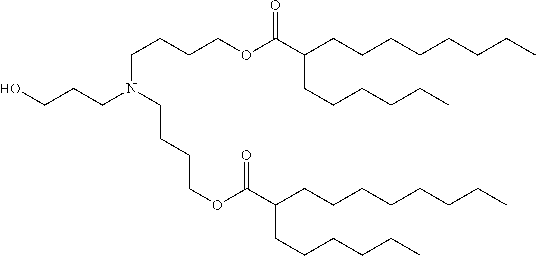 [SMILES: CCCCCCCCC(CCCCCC)C(=O)OCCCCN(CCCO)CCCCOC(=O)C(CCCCCC)CCCCCCCC] | F |
| III-6 | 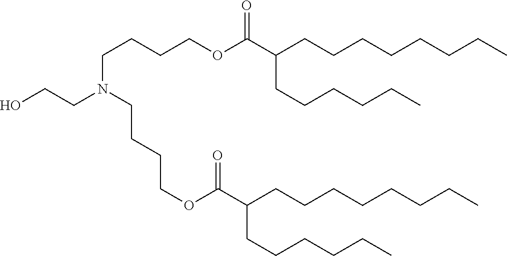 [SMILES: CCCCCCCCC(CCCCCC)C(=O)OCCCCN(CCO)CCCCOC(=O)C(CCCCCC)CCCCCCCC] | F |
| III-7 | 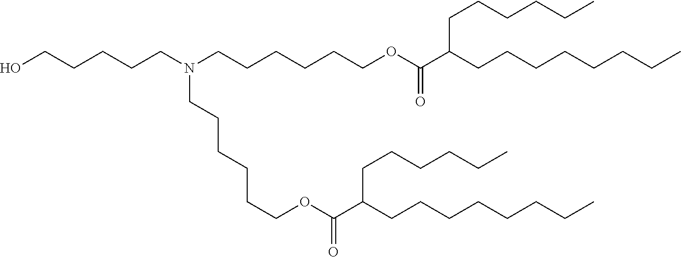 [SMILES: CCCCCCCCC(CCCCCC)C(=O)OCCCCCCN(CCCCCO)CCCCCCOC(=O)C(CCCCCC)CCCCCCCC] | F |
| III-8 | 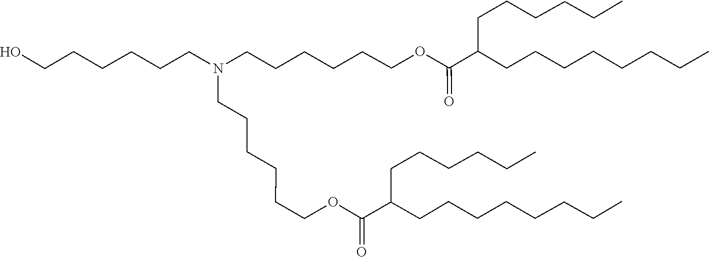 [SMILES: CCCCCCCCC(CCCCCC)C(=O)OCCCCCCN(CCCCCCO)CCCCCCOC(=O)C(CCCCCC)CCCCCCCC] | F |
| III-9 | 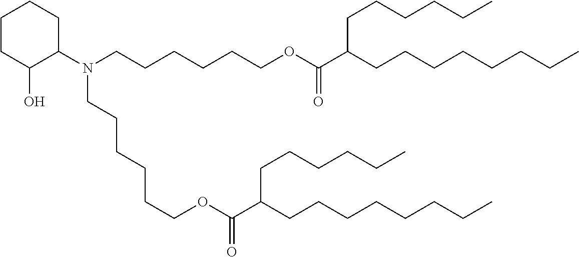 [SMILES: CCCCCCCCC(CCCCCC)C(=O)OCCCCCCN(CCCCCCOC(=O)C(CCCCCC)CCCCCCCC)C1CCCCC1O] | F |
| III-10 | 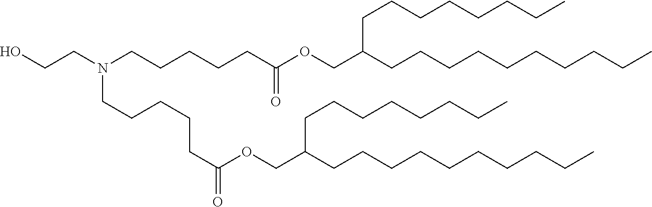 [SMILES: CCCCCCCCCCC(CCCCCCCC)COC(=O)CCCCCN(CCO)CCCCCC(=O)OCC(CCCCCCCC)CCCCCCCCCC] | F |
| III-11 | 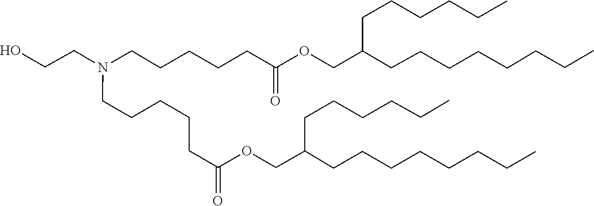 [SMILES: CCCCCCCCC(CCCCCC)COC(=O)CCCCCN(CCO)CCCCCC(=O)OCC(CCCCCC)CCCCCCCC] | F |
| III-12 | 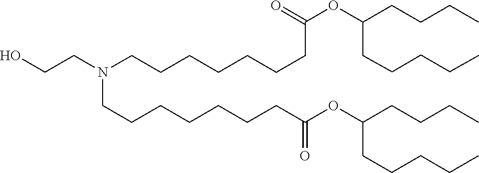 [SMILES: CCCCCC(CCCC)OC(=O)CCCCCCCN(CCO)CCCCCCCC(=O)OC(CCCC)CCCCC] | F |
| III-13 | 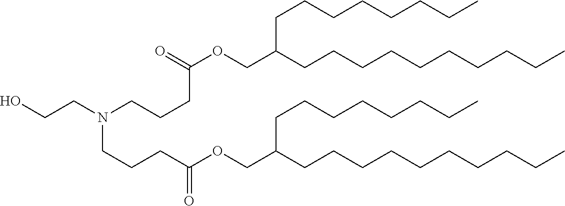 [SMILES: CCCCCCCCCCC(CCCCCCCC)COC(=O)CCCN(CCO)CCCC(=O)OCC(CCCCCCCC)CCCCCCCCCC] | F |
| III-14 | 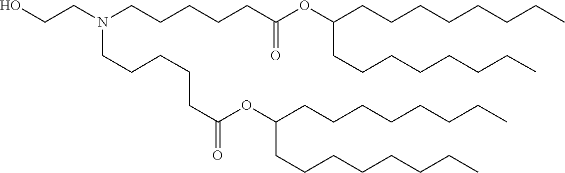 [SMILES: CCCCCCCCC(CCCCCCCC)OC(=O)CCCCCN(CCO)CCCCCC(=O)OC(CCCCCCCC)CCCCCCCC] | F |
| III-15 | 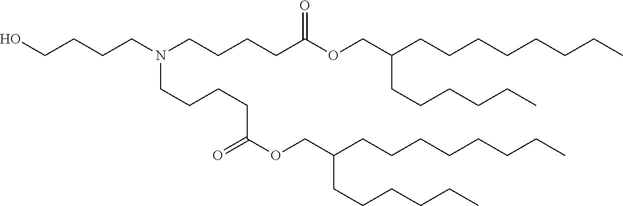 [SMILES: CCCCCCCCC(CCCCCC)COC(=O)CCCCN(CCCCO)CCCCC(=O)OCC(CCCCCC)CCCCCCCC] | F |
| III-16 | 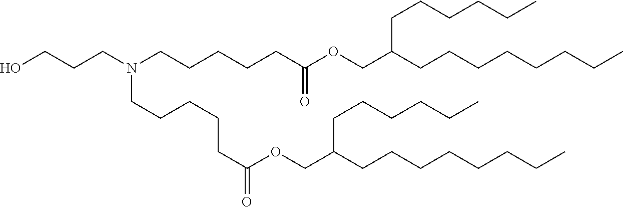 [SMILES: CCCCCCCCC(CCCCCC)COC(=O)CCCCCN(CCCO)CCCCCC(=O)OCC(CCCCCC)CCCCCCCC] | F |
| III-17 | 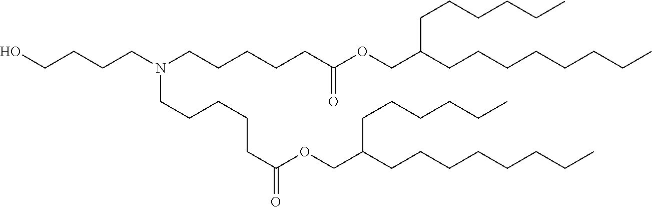 [SMILES: CCCCCCCCC(CCCCCC)COC(=O)CCCCCN(CCCCO)CCCCCC(=O)OCC(CCCCCC)CCCCCCCC] | F |
| III-18 | 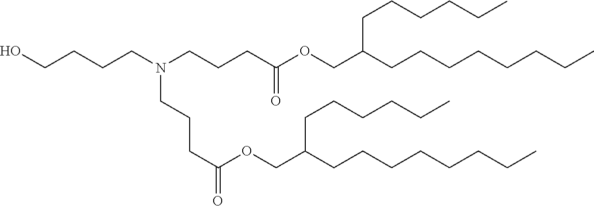 [SMILES: CCCCCCCCC(CCCCCC)COC(=O)CCCN(CCCCO)CCCC(=O)OCC(CCCCCC)CCCCCCCC] | F |
| III-19 | 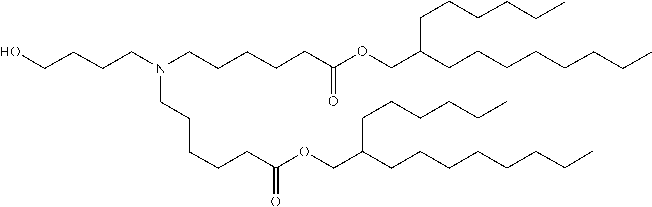 [SMILES: CCCCCCCCC(CCCCCC)COC(=O)CCCCCN(CCCCO)CCCCCC(=O)OCC(CCCCCC)CCCCCCCC] | F |
| III-20 | 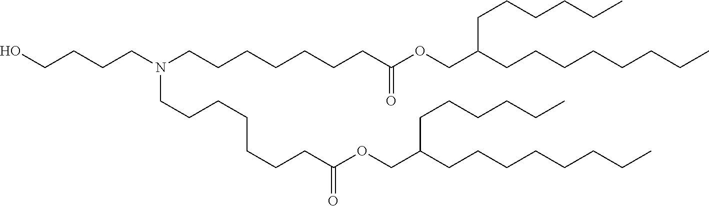 [SMILES: CCCCCCCCC(CCCCCC)COC(=O)CCCCCCCN(CCCCO)CCCCCCCC(=O)OCC(CCCCCC)CCCCCCCC] | F |
| III-21 | 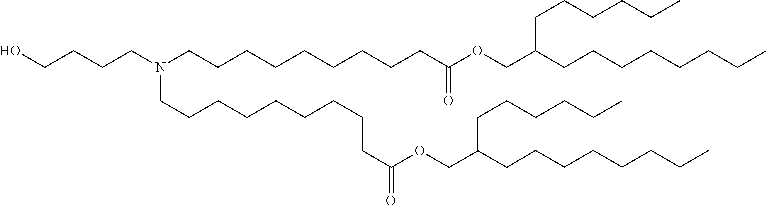 [SMILES: CCCCCCCCC(CCCCCC)COC(=O)CCCCCCCCCN(CCCCO)CCCCCCCCCC(=O)OCC(CCCCCC)CCCCCCCC] | F |
| III-22 | 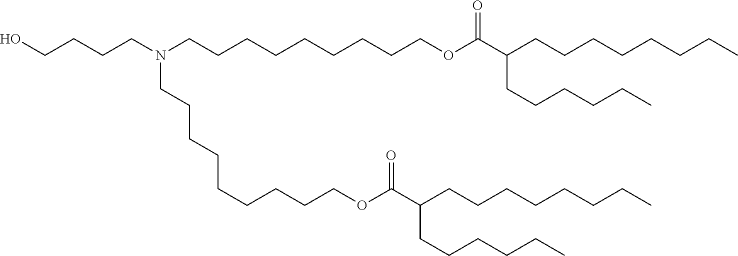 [SMILES: CCCCCCCCC(CCCCCC)C(=O)OCCCCCCCCCN(CCCCO)CCCCCCCCCOC(=O)C(CCCCCC)CCCCCCCC] | F |
| III-23 | 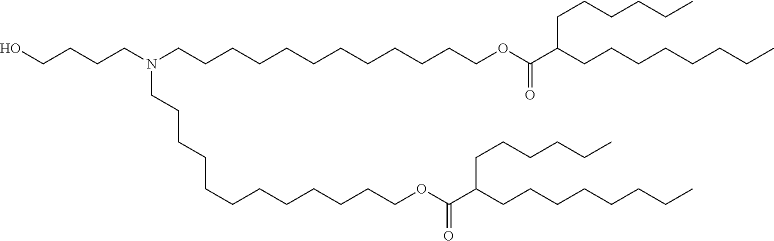 [SMILES: CCCCCCCCC(CCCCCC)C(=O)OCCCCCCCCCCCCN(CCCCO)CCCCCCCCCCCCOC(=O)C(CCCCCC)CCCCCCCC] | F |
| III-24 | 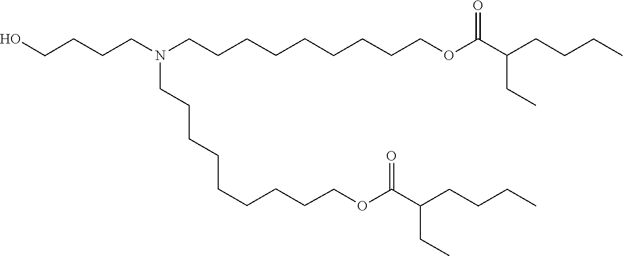 [SMILES: CCCCC(CC)C(=O)OCCCCCCCCCN(CCCCO)CCCCCCCCCOC(=O)C(CC)CCCC] | F |
| III-25 | 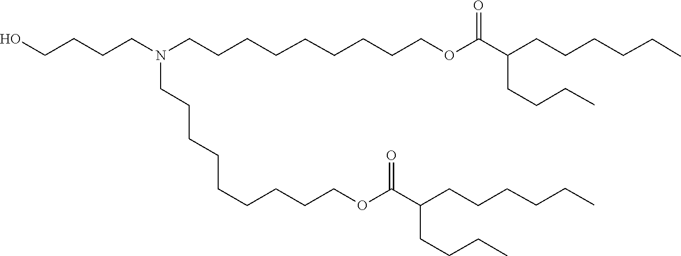 [SMILES: CCCCCCC(CCCC)C(=O)OCCCCCCCCCN(CCCCO)CCCCCCCCCOC(=O)C(CCCC)CCCCCC] | F |
| III-26 | 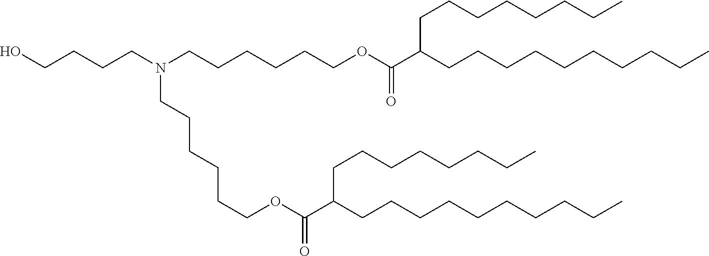 [SMILES: CCCCCCCCCCC(CCCCCCCC)C(=O)OCCCCCCN(CCCCO)CCCCCCOC(=O)C(CCCCCCCC)CCCCCCCCCC] | F |
| III-27 | 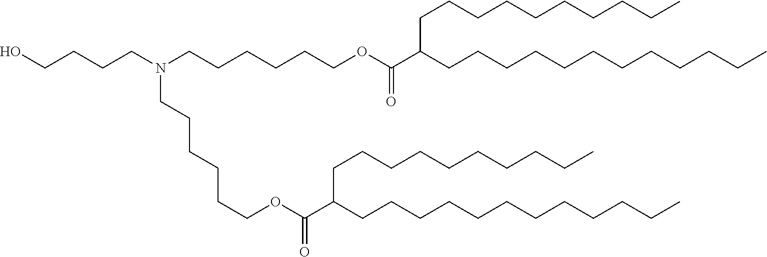 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] | F |
| III-28 | 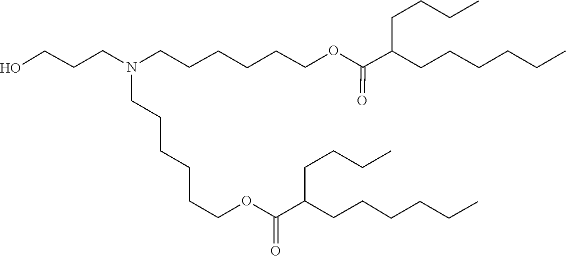 [SMILES: CCCCCCC(CCCC)C(=O)OCCCCCCN(CCCO)CCCCCCOC(=O)C(CCCC)CCCCCC] | F |
| III-29 | 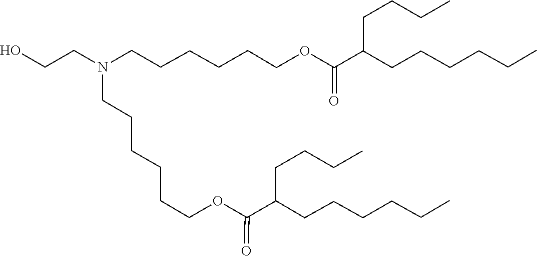 [SMILES: CCCCCCC(CCCC)C(=O)OCCCCCCN(CCO)CCCCCCOC(=O)C(CCCC)CCCCCC] | F |
| III-30 | 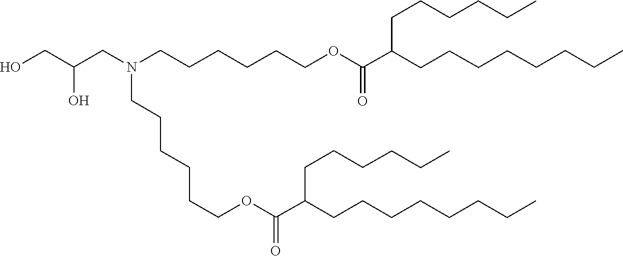 [SMILES: CCCCCCCCC(CCCCCC)C(=O)OCCCCCCN(CCCCCCOC(=O)C(CCCCCC)CCCCCCCC)CC(O)CO] | F |
| III-31 | 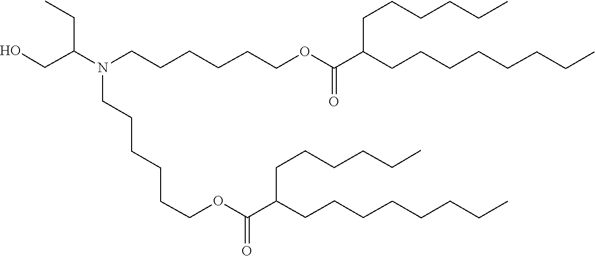 [SMILES: CCCCCCCCC(CCCCCC)C(=O)OCCCCCCN(CCCCCCOC(=O)C(CCCCCC)CCCCCCCC)C(CC)CO] | F |
| III-32 | 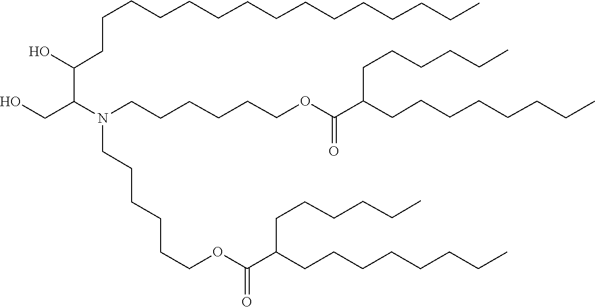 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] | F |
| III-33 | 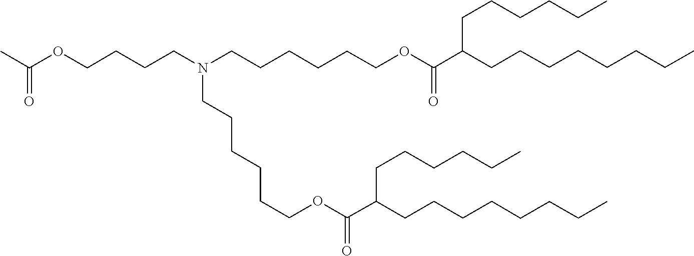 [SMILES: CCCCCCCCC(CCCCCC)C(=O)OCCCCCCN(CCCCCCOC(=O)C(CCCCCC)CCCCCCCC)CCCCOC(C)=O] | F |
| III-34 | 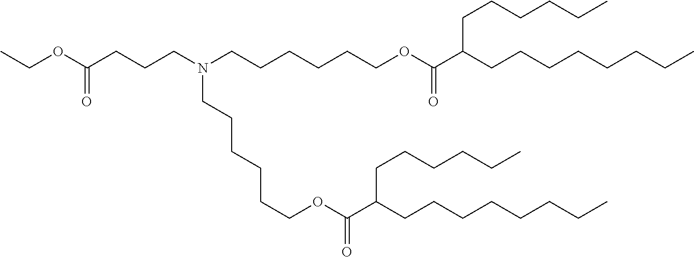 [SMILES: CCCCCCCCC(CCCCCC)C(=O)OCCCCCCN(CCCCCCOC(=O)C(CCCCCC)CCCCCCCC)CCCC(=O)OCC] | F |
| III-35 | 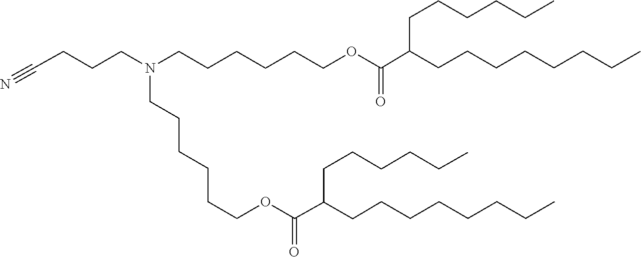 [SMILES: CCCCCCCCC(CCCCCC)C(=O)OCCCCCCN(CCCC#N)CCCCCCOC(=O)C(CCCCCC)CCCCCCCC] | F |
| III-36 | 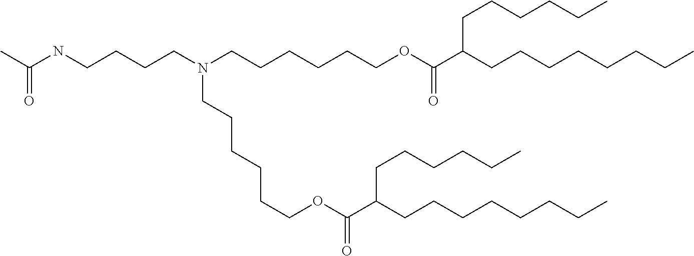 | F |

In some embodiments, the LNPs comprise a lipid of Formula (III), a nucleoside-modified RNA and one or more excipient selected from neutral lipids, steroids and pegylated lipids. In some embodiments the lipid of Formula (III) is compound III-3. In some embodiments the lipid of Formula (III) is compound III-7.

In some embodiments, the cationic lipid is present in the LNP in an amount from about 30 to about 95 mole percent. In one embodiment, the cationic lipid is present in the LNP in an amount from about 30 to about 70 mole percent. In one embodiment, the cationic lipid is present in the LNP in an amount from about 40 to about 60 mole percent. In one embodiment, the cationic lipid is present in the LNP in an amount of about 50 mole percent. In one embodiment, the LNP comprises only cationic lipids.

In some embodiments, the LNP comprises one or more additional lipids which stabilize the formation of particles during their formation.

Suitable stabilizing lipids include neutral lipids and anionic lipids.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1.

In various embodiments, the LNPs further comprise a steroid or steroid analogue. A “steroid” is a compound comprising the following carbon skeleton:

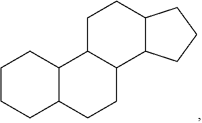 [SMILES: C1CCC2C(C1)CCC1C3CCCC3CCC21]

In some embodiments, the steroid or steroid analogue is cholesterol. In some of these embodiments, the molar ratio of the cationic lipid (e.g., lipid of Formula (I)) to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In some embodiments, the LNP comprises glycolipids (e.g., monosialoganglioside GM₁). In some embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In some embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In various embodiments, the molar ratio of the cationic lipid to the pegylated lipid ranges from about 100:1 to about 25:1.

In some embodiments, the LNPs comprise a pegylated lipid having the following structure (IV):

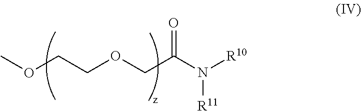

or a pharmaceutically acceptable salt, tautomer or stereoisomer thereof, wherein:

R¹⁰ and R¹¹ are each independently a straight or branched, saturated or unsaturated alkyl chain containing from 10 to 30 carbon atoms, wherein the alkyl chain is optionally interrupted by one or more ester bonds; and

z has mean value ranging from 30 to 60.

In some of the foregoing embodiments of the pegylated lipid (IV), R¹⁰ and R¹¹ are not both n-octadecyl when z is 42. In some other embodiments, R¹⁰ and R¹¹ are each independently a straight or branched, saturated or unsaturated alkyl chain containing from 10 to 18 carbon atoms. In some embodiments, R¹⁰ and R¹¹ are each independently a straight or branched, saturated or unsaturated alkyl chain containing from 12 to 16 carbon atoms. In some embodiments, R¹⁰ and R¹¹ are each independently a straight or branched, saturated or unsaturated alkyl chain containing 12 carbon atoms. In some embodiments, R¹⁰ and R¹¹ are each independently a straight or branched, saturated or unsaturated alkyl chain containing 14 carbon atoms. In other embodiments, R¹⁰ and R¹¹ are each independently a straight or branched, saturated or unsaturated alkyl chain containing 16 carbon atoms. In still more embodiments, R¹⁰ and R¹¹ are each independently a straight or branched, saturated or unsaturated alkyl chain containing 18 carbon atoms. In still other embodiments, R¹⁰ is a straight or branched, saturated or unsaturated alkyl chain containing 12 carbon atoms and R¹¹ is a straight or branched, saturated or unsaturated alkyl chain containing 14 carbon atoms.

In various embodiments, z spans a range that is selected such that the PEG portion of (II) has an average molecular weight of about 400 to about 6000 g/mol. In some embodiments, the average z is about 45.

In other embodiments, the pegylated lipid has one of the following structures:

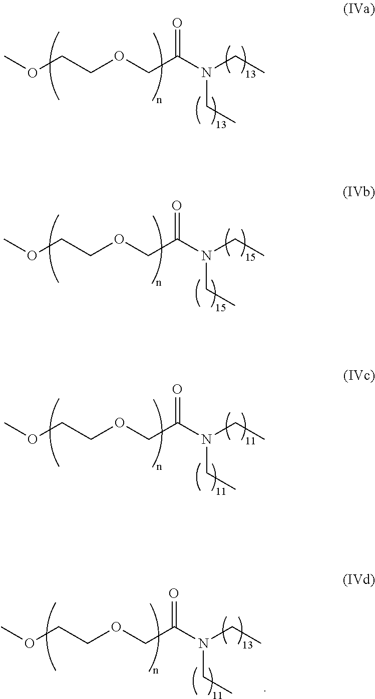

wherein n is an integer selected such that the average molecular weight of the pegylated lipid is about 2500 g/mol.

In some embodiments, the additional lipid is present in the LNP in an amount from about 1 to about 10 mole percent. In one embodiment, the additional lipid is present in the LNP in an amount from about 1 to about 5 mole percent. In one embodiment, the additional lipid is present in the LNP in about 1 mole percent or about 1.5 mole percent.

In some embodiments, the LNPs comprise a lipid of Formula (I), a nucleoside-modified RNA, a neutral lipid, a steroid and a pegylated lipid. In some embodiments the lipid of Formula (I) is compound I-6. In different embodiments, the neutral lipid is DSPC. In other embodiments, the steroid is cholesterol. In still different embodiments, the pegylated lipid is compound IVa.

In some embodiments, the LNP comprises one or more targeting moieties, which are capable of targeting the LNP to a cell or cell population. For example, in one embodiment, the targeting moiety is a ligand, which directs the LNP to a receptor found on a cell surface.

In some embodiments, the LNP comprises one or more internalization domains. For example, in one embodiment, the LNP comprises one or more domains, which bind to a cell to induce the internalization of the LNP. For example, in one embodiment, the one or more internalization domains bind to a receptor found on a cell surface to induce receptor-mediated uptake of the LNP. In some embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo.

Other exemplary LNPs and their manufacture are described in the art, for example in U.S. Patent Application Publication No. US20120276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference in their entirety.

The following Reaction Schemes illustrate methods to make lipids of Formula (I), (II) or (III).

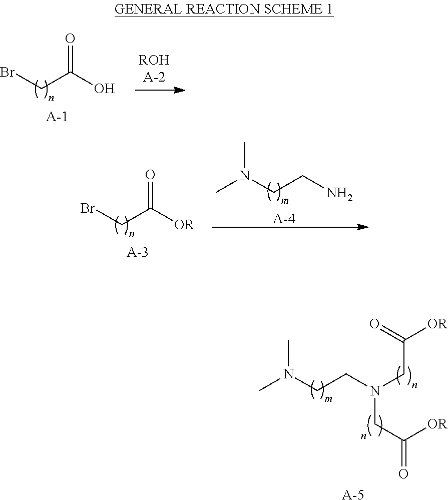

Embodiments of the lipid of Formula (I) (e.g., compound A-5) can be prepared according to General Reaction Scheme 1 (“Method A”), wherein R is a saturated or unsaturated C₁-C₂₄ alkyl or saturated or unsaturated cycloalkyl, m is 0 or 1 and n is an integer from 1 to 24. Referring to General Reaction Scheme 1, compounds of structure A-1 can be purchased from commercial sources or prepared according to methods familiar to one of ordinary skill in the art. A mixture of A-1, A-2 and DMAP is treated with DCC to give the bromide A-3. A mixture of the bromide A-3, a base (e.g., N,N-diisopropylethylamine) and the N,N-dimethyldiamine A-4 is heated at a temperature and time sufficient to produce A-5 after any necessarily workup and or purification step.

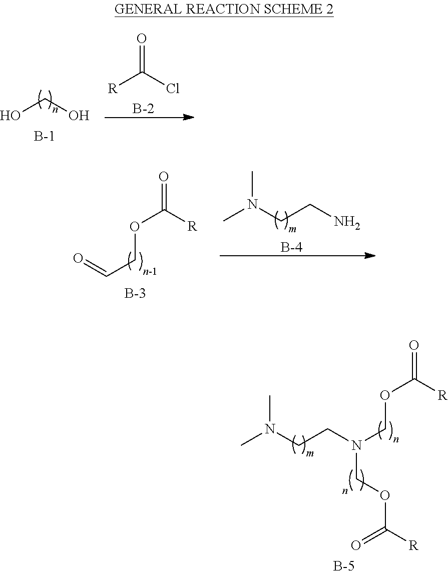

Other embodiments of the compound of Formula (I) (e.g., compound B-5) can be prepared according to General Reaction Scheme 2 (“Method B”), wherein R is a saturated or unsaturated C₁-C₂₄ alkyl or saturated or unsaturated cycloalkyl, m is 0 or 1 and n is an integer from 1 to 24. As shown in General Reaction Scheme 2, compounds of structure B-1 can be purchased from commercial sources or prepared according to methods familiar to one of ordinary skill in the art. A solution of B-1 (1 equivalent) is treated with acid chloride B-2 (1 equivalent) and a base (e.g., triethylamine). The crude product is treated with an oxidizing agent (e.g., pyridinum chlorochromate) and intermediate product B-3 is recovered. A solution of crude B-3, an acid (e.g., acetic acid), and N,N-dimethylaminoamine B-4 is then treated with a reducing agent (e.g., sodium triacetoxyborohydride) to obtain B-5 after any necessary work up and/or purification.

It should be noted that although starting materials A-1 and B-1 are depicted above as including only saturated methylene carbons, starting materials which include carbon-carbon double bonds may also be employed for preparation of compounds which include carbon-carbon double bonds.

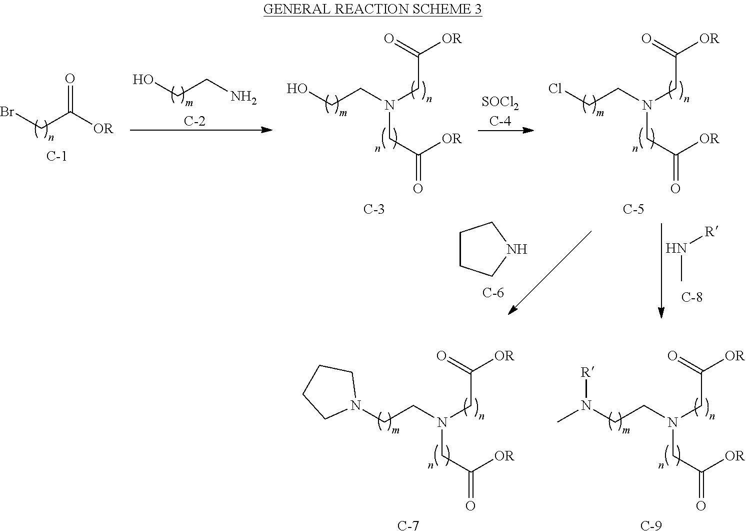

Different embodiments of the lipid of Formula (I) (e.g., compound C-7 or C9) can be prepared according to General Reaction Scheme 3 (“Method C”), wherein R is a saturated or unsaturated C₁-C₂₄ alkyl or saturated or unsaturated cycloalkyl, m is 0 or 1 and n is an integer from 1 to 24. Referring to General Reaction Scheme 3, compounds of structure C-1 can be purchased from commercial sources or prepared according to methods familiar to one of ordinary skill in the art.

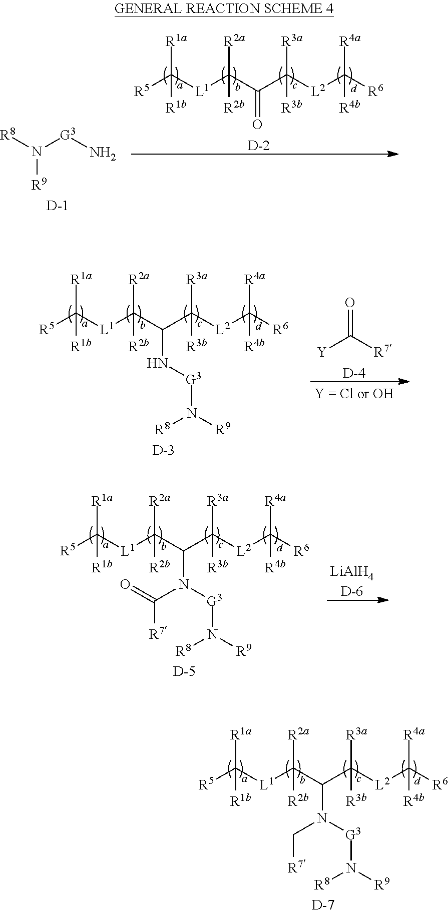

Embodiments of the compound of Formula (II) (e.g., compounds D-5 and D-7) can be prepared according to General Reaction Scheme 4 (“Method D”), wherein R^{1a}, R^{1b}, R^{2a}, R^{2b}, R^{3a}, R^{3b}, R^{4a}, R^{4b}, R⁵, R⁶, R⁸, R⁹, L¹, L², G¹, G², G³, a, b, c and d are as defined herein, and R^{7′} represents R⁷ or a C₃-C₁₉ alkyl. Referring to General Reaction Scheme 1, compounds of structure D-1 and D-2 can be purchased from commercial sources or prepared according to methods familiar to one of ordinary skill in the art. A solution of D-1 and D-2 is treated with a reducing agent (e.g., sodium triacetoxyborohydride) to obtain D-3 after any necessary work up. A solution of D-3 and a base (e.g. trimethylamine, DMAP) is treated with acyl chloride D-4 (or carboxylic acid and DCC) to obtain D-5 after any necessary work up and/or purification. D-5 can be reduced with LiAlH4 D-6 to give D-7 after any necessary work up and/or purification.

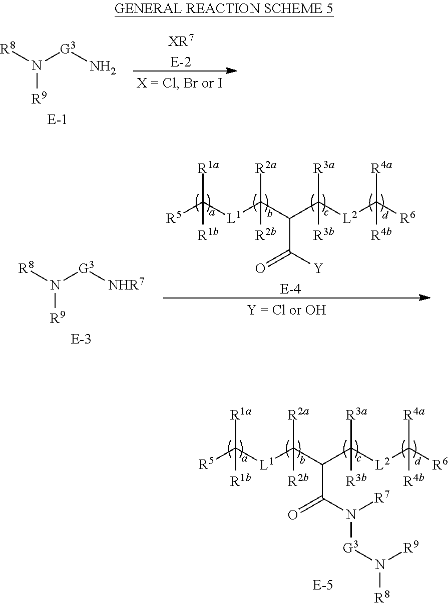

Embodiments of the lipid of Formula (II) (e.g., compound E-5) can be prepared according to General Reaction Scheme 5 (“Method E”), wherein R^{1a}, R^{1b}, R^{2a}, R^{2b}, R^{3a}, R^{3b}, R^{4a}, R^{4b}, R⁵, R⁶, R⁷, R⁸, R⁹, L¹, L², G³, a, b, c and d are as defined herein. Referring to General Reaction Scheme 2, compounds of structure E-1 and E-2 can be purchased from commercial sources or prepared according to methods familiar to one of ordinary skill in the art. A mixture of E-1 (in excess), E-2 and a base (e.g., potassium carbonate) is heated to obtain E-3 after any necessary work up. A solution of E-3 and a base (e.g. trimethylamine, DMAP) is treated with acyl chloride E-4 (or carboxylic acid and DCC) to obtain E-5 after any necessary work up and/or purification.

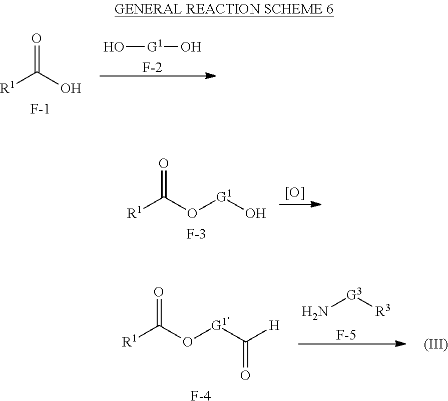

General Reaction Scheme 6 provides an exemplary method (Method F) for preparation of Lipids of Formula (III). G¹, G³, R¹ and R³ in General Reaction Scheme 6 are as defined herein for Formula (III), and G1′ refers to a one-carbon shorter homologue of G1. Compounds of structure F-1 are purchased or prepared according to methods known in the art. Reaction of F-1 with diol F-2 under appropriate condensation conditions (e.g., DCC) yields ester/alcohol F-3, which can then be oxidized (e.g., PCC) to aldehyde F-4. Reaction of F-4 with amine F-5 under reductive amination conditions yields a lipid of Formula (III).

It should be noted that various alternative strategies for preparation of lipids of Formula (III) are available to those of ordinary skill in the art. For example, other lipids of Formula (III) wherein L¹ and L² are other than ester can be prepared according to analogous methods using the appropriate starting material. Further, General Reaction Scheme 6 depicts preparation of a lipids of Formula (III), wherein G¹ and G² are the same; however, this is not a required aspect of the invention and modifications to the above reaction scheme are possible to yield compounds wherein G¹ and G² are different.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient, which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers. In some embodiments, the formulation may comprise dry particles which comprise the active ingredient and which have a diameter in the range from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some instances having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Scaffold Compositions

In one aspect, the present invention provides a composition comprising a scaffold comprising one or more nucleic acid molecules encoding one or more growth factors, or fragments or variants thereof, as described herein. For example, in one embodiment, the scaffold comprises one or more nucleoside modified RNA encoding one or more growth factors, or fragments or variants thereof, as described herein. In one embodiment, the scaffold comprises one or more LNPs encapsulating one or more nucleoside modified RNA encoding one or more growth factors, or fragments or variants thereof, as described herein. Exemplary scaffold compositions include, but are not limited to, hydrogels, electrospun scaffolds, and combinations thereof. In certain embodiments, the scaffold is biocompatible. In certain embodiments, the scaffold is biodegradable. In certain embodiments, the scaffold comprises one or more cells embedded within the scaffold or cultured along the surface of the scaffold. For example, in one embodiment, the scaffold comprises periodontal ligament cells, or precursors thereof.

In certain embodiments, the scaffold comprises one or more extracellular matrix material and/or blends of naturally occurring extracellular matrix material, including but not limited to collagen, fibrin, fibrinogen, thrombin, elastin, laminin, fibronectin, hyaluronic acid, chondroitin 4-sulfate, chondroitin 6-sulfate, dermatan sulfate, heparin sulfate, heparin, and keratan sulfate, proteoglycans, and combinations thereof. Some collagens that may be beneficial include but are not limited to collagen types I, II, III, IV, V, VI, VII, VIII, IX, X, XI, XII, XIII, XIV, XV, XVI, XVII, XVIII, and XIX. These proteins may be in any form, including but not limited to native and denatured forms. The scaffolds can further comprise one or more carbohydrates such as chitin, chitosan, alginic acids, and alginates such as calcium alginate and sodium alginate. These materials may be isolated from plant products, humans or other organisms or cells or synthetically manufactured. In some embodiments, the scaffolds can further comprise decellularized or demineralized tissue. In some embodiments, the scaffolds can further comprise synthetic peptides Also contemplated are crude extracts of tissue, extracellular matrix material, or extracts of non-natural tissue, alone or in combination. Extracts of biological materials, including but are not limited to cells, tissues, organs, and tumors may also be included.

In one embodiment, the scaffold comprises a polymer. Suitable polymers include but are not limited to: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO) and polyorthoesters or any other similar synthetic polymers that may be developed that are biologically compatible. Polymers with cationic moieties can also be used, such as poly(allyl amine), poly(ethylene imine), poly(lysine), and poly(arginine). The polymers may have any molecular structure including, but not limited to, linear, branched, graft, block, star, comb, and dendrimer structures.

In various embodiments, the scaffolds can include one or more therapeutics. The therapeutics can be natural or synthetic drugs, including but not limited to: analgesics, anesthetics, antifungals, antibiotics, anti-inflammatories, nonsteroidal anti-inflammatory drugs (NSAIDs), antimicrobials, antiseptics, antivirals, a colored or fluorescent imaging agent, corticoids (such as steroids), enzymes, growth factors, hormones, minerals, nutritional supplements, vitamins, and the like. The therapeutic agent may also be other small organic molecules, naturally isolated entities or their analogs, organometallic agents, chelated metals or metal salts, peptide-based drugs, or peptidic or non-peptidic receptor targeting or binding agents. In some embodiments, the scaffolds can further comprise a delivery vehicle, such as nanoparticles, microparticles, liposomes, viral and non-viral transfection systems.

In one embodiment, the scaffold comprises a cell or cell population. The cell or cell population can include any cell that contributes to periodontal tissue and/or bone regeneration. Non-limiting examples of cells include periodontal ligament cells, periodontal ligament stem cells, osteoblasts, osteoprogenitor cells, and differentiated and undifferentiated stem cells. In some embodiments, the population of cells is at least partially derived from a subject's own tissue. In some embodiments, the population of cells is at least partially derived from another subject within the same species as the treated subject. In some embodiments, the population of cells is at least partially derived from a mammalian species that is different from the subject. For example, the cells may be derived from organs of mammals such as humans, monkeys, dogs, cats, mice, rats, cows, horses, pigs, goats and sheep.

In certain embodiments, the cell or cell population is genetically modified. In certain embodiments, the cells are genetically modified to express one or more of the growth factors, described herein. In one embodiment, the cells are modified to comprise a nucleic acid molecule encoding the one or more growth factors described herein. For example, in one embodiment, the cells are modified to comprise one or more nucleoside modified RNA encoding one or more growth factors, or fragments or variants thereof.

### Treatment Methods

The present invention provides methods of inducing regeneration of periodontal tissue and/or bone in a subject comprising administering an effective amount of a composition comprising one or more isolated nucleic acids encoding one or more growth factors, or fragments or variants thereof. In one embodiment, the method treats or prevents gum disease, periodontitis, and/or bone defects in a subject.

In one embodiment, the composition is administered to a subject having periodontitis. In one embodiment, the composition is administered to a subject at risk for developing periodontitis. In one embodiment, the composition is administered to a subject having bone defects. In one embodiment, the composition is administered to a subject at risk for developing bone deficiencies. In one embodiment, the composition is administered to a subject having periodontitis or bone defects. In one embodiment, the composition is administered to a subject at risk for developing periodontitis or bone deficiencies. In certain embodiments, the subject has a bone defect, or is at risk for having a bone defect, that may be caused by any number of conditions or diseases. For example, in certain instances the subject has received a dental implant (peri-implant diseases), undergone a tooth extraction, experienced a traumatic injury, or has a bone defect caused by other diseases

In one embodiment, the method comprises administering a composition comprising one or more nucleoside-modified nucleic acid molecules encoding one or more growth factors, or fragments or variants thereof. In one embodiment, the method comprises administering a composition comprising a first nucleoside-modified nucleic acid molecule encoding a first growth factor, or a fragment or a variant thereof and a second nucleoside-modified nucleic acid molecule encoding a second growth factor, or a fragment or a variant thereof.

In one embodiment, the method comprises administering one or more compositions, each composition comprising one or more nucleoside-modified nucleic acid molecules encoding one or more growth factors, or fragments or variants thereof. In one embodiment, the method comprises administering a first composition comprising one or more nucleoside-modified nucleic acid molecules encoding one or more growth factors, or fragments or variants thereof; and administering a second composition comprising one or more nucleoside-modified nucleic acid molecules encoding one or more growth factors, or fragments or variants thereof.

In some embodiments, the method of the invention allows for sustained expression of the one or more growth factors, described herein, for at least several days following administration. In some embodiments, the method of the invention allows for sustained expression of the one or more growth factors, described herein, for at least 2 weeks following administration. In some embodiments, the method of the invention allows for sustained expression of the one or more growth factors, described herein, for at least 1 month following administration. However, the method, in some embodiments, also provides for transient expression, as in some embodiments, the nucleic acid is not integrated into the subject genome.

In some embodiments, the method comprises administering nucleoside-modified RNA, which provides stable expression of the one or more growth factors described herein. In some embodiments, administration of nucleoside-modified RNA results in little to no innate immune response, while inducing periodontal tissue and/or bone regeneration.

In some embodiments, the method provides sustained periodontal tissue and/or bone regeneration. For example, in some embodiments, the method provides sustained periodontal tissue and/or bone regeneration for more than 2 weeks. In some embodiments, the method provides sustained periodontal tissue and/or bone regeneration for 1 month or more. In some embodiments, the method provides sustained periodontal tissue and/or bone regeneration for 2 months or more. In some embodiments, the method provides sustained periodontal tissue and/or bone regeneration for 3 months or more. In some embodiments, the method provides sustained periodontal tissue and/or bone regeneration for 4 months or more. In some embodiments, the method provides sustained periodontal tissue and/or bone regeneration for 5 months or more. In some embodiments, the method provides sustained periodontal tissue and/or bone regeneration for 6 months or more. In some embodiments, the method provides sustained periodontal tissue and/or bone regeneration for 1 year or more.

In one embodiment, a single administration of the composition induces sustained periodontal tissue and/or bone regeneration for 1 month or more, 2 months or more, 3 months or more, 4 months or more, 5 months or more, 6 months or more, or 1 year or more.

Administration of the compositions of the invention in a method of treatment can be achieved in a number of different ways, using methods known in the art. In one embodiment, the method of the invention comprises systemic administration of the subject, including for example enteral or parenteral administration. In some embodiments, the method comprises oral delivery of the composition. In another embodiment, the method comprises intravenous delivery of the composition. In one embodiment, the method comprises injection of the composition into the periodontal tissue and/or bone of a subject. In one embodiment, the method comprises administration of a scaffold composition to the periodontal tissue and/or bone defects of a subject.

It will be appreciated that the composition of the invention may be administered to a subject either alone, or in conjunction with another agent.

The therapeutic and prophylactic methods of the invention thus encompass the use of pharmaceutical compositions encoding one or more growth factors, or fragments or variants thereof, described herein to practice the methods of the invention. The pharmaceutical compositions useful for practicing the invention may be administered to deliver a dose of from 1 ng/kg/day and 100 mg/kg/day. In one embodiment, the invention envisions administration of a dose, which results in a concentration of the compound of the present invention from 10 nM and 10 μM in a mammal.

Typically, dosages which may be administered in a method of the invention to a mammal, such as a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 0.1 μg to about 10 mg per kilogram of body weight of the mammal. In some embodiments, the dosage will vary from about 1 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months, several years, or even less frequently, such as every 10-20 years, 15-30 years, or even less frequently, such as every 50-100 years. The frequency of the dose will be readily apparent to the skilled artisan and will depend upon any number of factors, such as, but not limited to, the type and severity of the disease being treated, the type and age of the mammal, etc.

In one embodiment, the invention includes a method comprising administering one or more compositions encoding one or more growth factors, or fragments or variants thereof, described herein. In some embodiments, the method has an additive effect, wherein the overall effect of the administering the combination is approximately equal to the sum of the effects of administering each growth factor. In other embodiments, the method has a synergistic effect, wherein the overall effect of administering the combination is greater than the sum of the effects of administering each growth factor or adjuvant.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: The Use of Modified mRNA Encoding Platelet-Derived Growth Factor-BB as an Innovation in Periodontal Regeneration

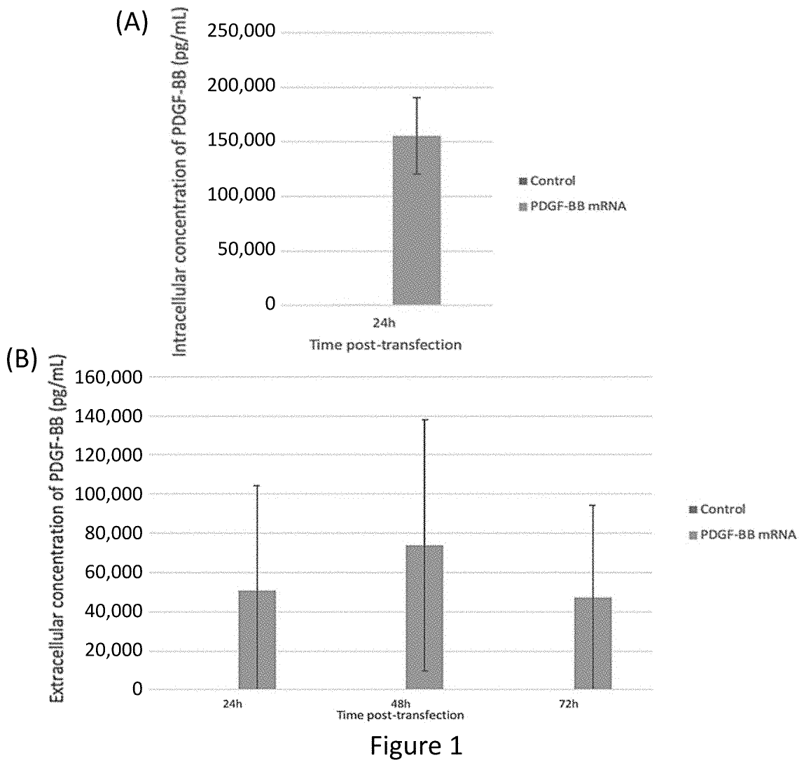

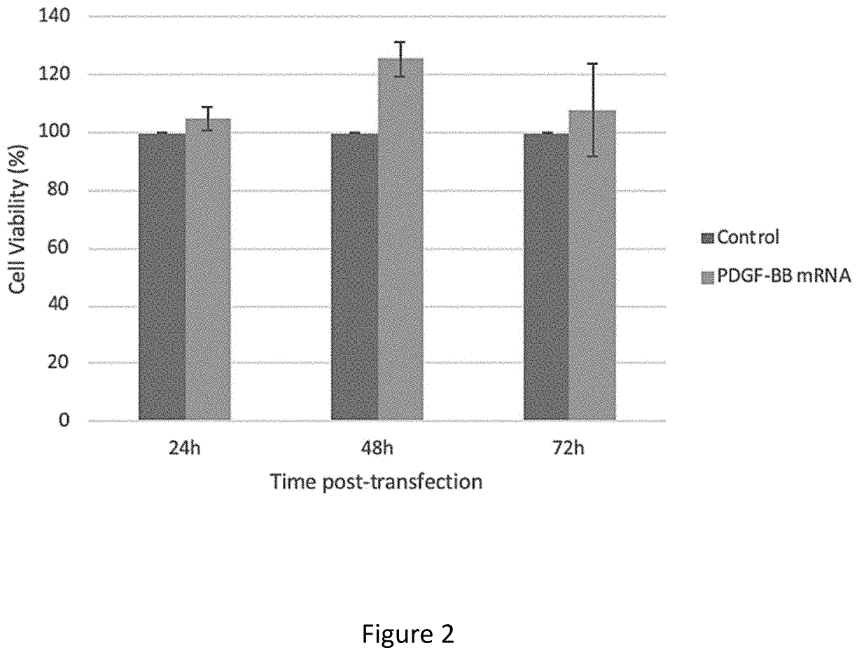

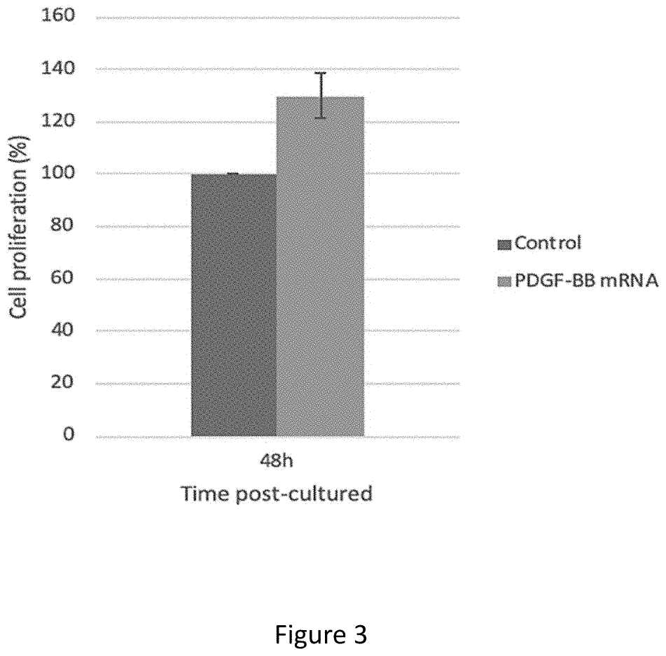

Since mRNA platforms have been recently introduced to several fields in medicine and the achievement of periodontal regeneration is currently unpredictable, the use of mRNA technology tends to be a promising approach to pursue the complete periodontal regeneration. The aims of this study are to learn if mRNA encoding platelet-derived growth factor-BB (PDGF-BB) induces PDGF-BB production in human periodontal ligament cells (PDLCs) as well as to investigate the effect of secreted PDGF on PDLC proliferation. PDLCs were obtained from extracted teeth of healthy periodontal patients. The modified N1-methylpseudouridine mRNA encoding PDGF-BB were transfected into PDLCs. The supernatants were collected from 24-, 48- and 72-h time points and the protein production was measured using ELISA. The viability of transfected PDLCs was also assessed. In addition, the supernatants collected at the 24-h timepoint were used for the proliferation assay using alamarBlue. The result showed that PDLCs, transfected with mRNA encoding PDGF-BB, produced higher levels of intracellular PDGF-BB than controls at 24 h (FIG. 1A). PDGF-BB was also detected in the supernatants started from 24 h and constantly secreted up to 72 h (FIG. 1B). The transfection of mRNA encoding PDGF-BB had no effect on PDLC viability (FIG. 2). The supernatants containing PDGF-BB were able to promote PDLC proliferation (FIG. 3). Thus, this demonstrates that this mRNA platform technology is applicable for periodontal tissue regeneration.

During the past decade, tissue engineering has been introduced as an innovation to regenerate the destructed periodontium. There are three essential constituents: stem cells, scaffolds and signaling molecules. First, stem cells can reproduce themselves and differentiate into various types of cells. Second, the scaffold is a construction or framework that allows target cells to attach, proliferate and differentiate into target tissue organs. Last, signaling molecules or growth factors are proteins that induce cells to proliferate and differentiate by interacting with their receptors (Taba et al. 2005, Orthodontics & Craniofacial Research, 8(4): 292-302; Smith, 2006, Nature, 441(7097): 1060).

Periodontal ligament cells are a major source of mesenchymal stem cells in the oral cavity which has a regenerative potential (Bartold et al., 2000, Periodontology, 86(2 Suppl): 5108-30). To enhance the success in periodontal regeneration, mesenchymal stem cells have been used in the combination with either scaffolds such as collagen, fibrin, hydrogel, and gelatin or non-scaffold materials such as cell sheets (Akizuki, et al., 2005, Journal of Periodontal Research, 40(3): 245-51). However, the uses of mesenchymal stem cells and cell sheets in this field are limited due to the lack of cell sources, and the fact that they are time consuming and complicated procedures.

Growth factors is considered as one of the developed therapies for periodontal regeneration by stimulating stem cell proliferation and differentiation. For decades, numerous growth factors were used in regeneration including fibroblast growth factor (FGFs), platelet derived growth factor (PDGF), insulin growth factor (IGF), vascular endothelial cell growth factor (VEGF), epidermal growth factor (EGF) and bone morphogenic proteins (BMPs) (Kao et al., 2009, Periodontology, 50: 127-53).

Platelet derived growth factors (PDGFs) are known as a group of growth factors promoting tissue regeneration and wound healing (Dereka et al., 2006). PDGF consists of 3 forms: PDGF-AA, PDGF-BB and PDGF-AB. The use of 0.3 mg/ml recombinant human PDGF-BB (rhPDGF-BB) positively impacted bone fill, bone height gain and clinical attachment gain in periodontal defects (Li et al, 2017, Scientific Reports, 7(1): 65). A clinical study was also shown that PDGF provided a comparable result in periodontal regeneration to GTR or bone grafts (Darby and Morris 2013, Journal of Periodontology, 84(4): 465-76). rhPDGF-BB has become commercially available as Regranex® and GEM 21S® for promoting soft tissue healing and periodontal bone regeneration, respectively (Solchaga et al., 2013, Journal of Tissue Engineering, 3(1): 2041731412442668). The Food and Drug Administration (FDA) also granted for the use of 0.03% PDGF-BB in combination with β-tricalcium phosphate synthetic bones to correct intrabony defects, furcation defects and gingival recession (Suarez-Lopez Del Amo et al., 2015, Biomed research international, 2015:957518). Even though PDGF-BB has potential in stimulating PDL stem cells to proliferate and differentiate, the leakage of protein from the treated area as well as its high cost are major limitations.

Apart from using recombinant growth factors, the use of DNA in gene therapy has been introduced in treating several diseases. The gene therapy can be performed by processing plasmid DNA or viral vector. Foreign DNA is delivered to the nucleus by passaging through the cell and nuclear membranes. Foreign DNA is integrated into the host genome and transgene expression is sustained even after host cells replicate (Kim and Eberwine, 2010, Analytical and Bioanalytical Chemistry, 397(8): 3173-8). For example, an adenovirus encoding PDGF-AA can transfer DNA and induce PDGF-AA protein production in gingival fibroblasts. (Chen and Giannobile 2002). Nonetheless, gene therapy should be used with caution due to safety issues including mutation and tumorigenesis (Kim and Eberwine, 2010, Analytical and Bioanalytical Chemistry, 397(8): 3173-8).

Even though various approaches are applied to treat this disease, unexpected outcomes of the treatment can occur depending upon the defect characteristics. DNA therapy also has an increased a risk of mutation. Using recombinant protein requires large amounts of protein and most of the time it remains in the tissue only a short period of time. Moreover, the treatment cost is considered as another limitation because most materials are expensive and required to be imported. In addition, these available techniques mentioned above were unable to achieve complete regeneration of the periodontium.

mRNA therapy is the use of specific mRNA delivered with a carrier into the cytoplasm of the patient's cell to achieve the process of translation for the desired protein. These proteins have functions or properties as signaling molecules or growth factors that induce cell proliferation and differentiation in the periodontal tissue and bone regeneration. The process of mRNA technology is an advanced innovation that will transform the medical treatment base with biotechnology for safe and affordable treatment. There are many advantages that are better than DNA therapy, such as good delivery efficiency and no risk of integration with the host genome (Kim and Eberwine, 2010, Analytical and Bioanalytical Chemistry, 397(8): 3173-8).

Currently, the use of mRNA has been developed by various methods to prevent mRNA degradation and increase the effectiveness in protein expression. The encapsulation of mRNA enhances its stability and helps in uptake of mRNA into the cell. The mRNA encapsulation or transfection can be performed in several forms such as lipids encapsulation (Mintzer and Simanek, 2009, Chemical reviews, 109(2): 259-302), polymers (Pack et al., 2005, Nature reviews drug discovery, 4(7): 581-93) and peptides (Martin and Rice, 2007, The AAPS Journal, 9(1): E18-29). The most reliable system for delivering mRNA to the cell is cationic lipid nanoparticles. These nanoparticles are easy to synthesize and import into target cells with specific ligands. A positive charge of cationic lipid is subjected to attach to the negative charge of mRNA, then, they aggregate into lipid nanoparticles. (Karikó et al., 2012, Molecular Therapy: the journal of the American Society of Gene Therapy, 20(5): 948-53; DeRosa et al., 2016, Gene Ther, 23(10): 699-707). Furthermore, increasing the stability of mRNA by gene sequence coding modification, such as 5′cap, 5 ‘ and 3’ UTR and the length of poly A, and especially chemical modification of nucleosides, leads to the improvement of protein production (Zhang et al., 2019, Tissue Engineering Part A, 25(1-2): 131-44).

Unfortunately, the limitation of mRNA in tissue regeneration is that mRNA has a potential to stimulate the immune system. The induction of the innate immune response to mRNA was an undesired effect (Goubau et al., 2013, Immunity, 38(5): 855-69). Since mRNA could be recognized as foreign bodies or viruses and be recognized by various receptors, the binding between receptors and mRNA led to cell activation, and finally inhibited protein formation (Pollard et al., 2013, Molecular Therapy, the journal of the American Society of Gene Therapy, 21(1): 251-9). Thus, an attempt to inhibit the immune activation by modifying the base part of the mRNA was suggested. Particularly, the base adjustment at pseudouridine or N-1 methylpseudouridine was able to inhibit the response of the innate immune system via type 1 interferon, and promote protein production (Karikó et al., 2008, Molecular Therapy, the journal of the American Society of Gene Therapy, 16(11): 1833-40; Andries et al., 2015, Journal of Controlled Release: official journal of the Controlled Release Society, 217: 337-44).

Thus, the advantages of mRNA technology are greater than stem cell, plasmid DNA and viral vector technologies described as follows. 1.) The mRNA technology is safe because mRNA is a transient genetic transporter degraded naturally. It also has no risk of integrating into host genome. 2.) The mRNA can uptake into the different cell types. Moreover, it can produce high protein levels. 3.) Unlike the use of a viral vector, mRNA technology can be used in people or animals without inducing an immune response. 4.) The ability to produce the large amounts of mRNA and good manufacturing practice (GMP) grade in vitro can be used in clinical setting. As mentioned above, it is possible to introduce the advanced innovation in the development of mRNA technology platform for the medical tissue regeneration. This study describes an in vitro design of the mRNA biometrics by selecting an mRNA which encodes PDGF-BB and modifying the bases which are the most effective in the expression in human periodontal ligament cells. This study further investigates the bioactivity such as human periodontal ligament cell proliferation, Vascular endothelial growth factor (VEGF) production and the ability to induce tube formation by endothelial cells. Experiments were conducted to examine whether mRNA encoding PDGF-BB induces PDGF-BB production in PDLCs and investigate the effect of PDGF-BB induced by mRNA encoding PDGF-BB transfection on PDLC proliferation and viability.

The methods and materials employed in these experiments are now described.

Construction of mRNA Encoding PDGF-BB

The nucleotide sequence of human PDGF-BB was designed and the N1-methylpseudouridine-modified mRNA was synthesized (Pardi et al., 2017, Nature Communications, 8: 14630; Pardi et al., 2018, Nature Communications, 9(1): 3361; Pardi et al., 2018, Nature Reviews Drug Discovery, 17(4): 261-79, each of which is incorporated by reference in its entirety).

### Medium and Reagents

Minimum Essential Medium with Alpha modification (Alpha MEM) supplemented with 10% heat-inactivated fetal calf serum, 2 mM GlutaMax-I, 100 U/ml penicillin, 100 μg/ml streptomycin and 5 μg/ml amphotericin B (Life Technologies) was used throughout the study. Opti-MEM I, Lipofectamine 2000 were purchased from Invitrogen. Human recombinant PDGF-BB was obtained from R&D Systems.

### Cells Isolation and Culture

All participants were provided written informed consent. Human periodontal ligament tissues from 10 healthy periodontal patients (age 15-35 years) undergoing wisdom tooth extraction or tooth extraction due to orthodontic reason were obtained. PDLCs were separated from the tooth by enzyme-digestion method. Briefly, the tooth was extensively washed twice with Dulbecco's phosphate-buffered saline (DPBS) and the PDL tissues were scraped out from the middle third of the root under a sterile condition to avoid the contamination from gingival or periapical granulation tissues. Then, PDL tissues were minced into a fragment of 1-2 mm² and digested with a solution of 2 mg/ml collagenase and 2 mg/ml dispase for 60 minutes at 37° C. and then filtered through a 70-μm cell strainer. The pass-through was washed twice with culture medium. The PDLCs were cultured with the medium at 37° C. in a humidified atmosphere of 5% CO₂. After a confluent monolayer of cells was reached, PDLCs were trypsinized, washed and then sub-cultured. The cells from 3^{rd} to 8^{th} passages from 3 different donors were used in this study (Iwata et al., 2010, Journal of Clinical Periodontology, 37(12): 1088-99).

### In Vitro Cell Transfection and Expression/Secretion of PDGF-BB Protein

For in vitro transfection of cells, non-modified and modified mRNA encoding PDGF-BB was complexed with Lipofectamine® 2000 (Invitrogen) and transfected into PDLCs—according to manufacturer's instructions. To analyze the PDGF-BB protein production and secretion levels, the transfected cells were cultured for 24-72 hrs. Supernatants and cells were collected at 24, 48 and 72 hrs. The cells were lysed using RIPA buffer (Pierce® RIPA buffer, ThermoFisher Scientific) and the lysates were stored for further analysis. PDGF-BB production and secretion were analyzed using ELISA (Quantikine®, R&D System, Minnesota, USA).

### Cell Proliferation and Toxicity

To determine PDLC proliferation following mRNA encoded PDGF transfection, PDLCs were plated in 96-well plate (3×10³ cells per well) and either control medium, supernatants of transfected PDLCs and recombinant PDGF-BB were added. After a 24-hour incubation, 10% Alamar Blue solution (alamarBlue®, BIO-RAD, California, USA) was added. The cell proliferation ability was measured by a microplate reader at absorbance of 570 nm (Epoch™, Biotek™, Vermont, USA) according to the manufacturer instruction.

To analyze cell toxicity, PDLCs with either mRNA complexed with transfecting agent or transfecting agent alone were cultured with 10% alamarBlue solution then incubated at 37° C. in humidified atmosphere of 5% CO₂. After 4 hours, cell culture supernatants were measured at absorbance of 570 nm using microplate reader.

The results of the experiments are now described.

### Intracellular and Extracellular PDGF-BB Protein Production

The amount of intracellular and extracellular PDGF-BB protein secretion was determined from periodontal ligament cell culture which was transfected with either N1-methylpseudouridine mRNA encoding PDG-BB with Lipofectamine 2000 or Lipofectamine 2000 alone. After transfection for 24 hours, periodontal ligament cells were collected and digested to measure the amount of intracellular protein production. The result showed that the mRNA PDGF-BB transfected periodontal ligament cells were able to produce higher intracellular proteins than the control at 24 hours, with the mean of 155540.3 picograms per milliliter (FIG. 1A).

The supernatants from the cultures were collected at 24, 48 and 72 hours and taken to measure the amount of extracellular PDGF-BB protein secretion. The protein secretion was detected at 24 hours and continuously secretion up to 72 hours, and the protein levels were higher than the control group. The mean concentrations of PDGF-BB were 50,533.33, 73,716.67 and 76,450 picograms per milliliter at 24, 48 and 72 hours, respectively (FIG. 1B).

Cell Viability after Transfection with N1-Methylpseudouridine mRNA Encoding PDGF-BB with Lipofectamine 2000

The PDCLs were transfected with modified mRNA encoded PDGF-BB, and the cells were then harvested at 24, 48 and 72 hours for analyzing viability using alamarBlue assay. The results showed that the mRNA encoded PDGF-BB did not affect the viability of periodontal cells. In addition, it showed that cell viability was still greater than 90 percent after 72 hours and similar to the controls (FIG. 2).

### Biological Activity of Translated PDGF-BB Protein

The periodontal cells were incubated with the clear part of the periodontal cells transfected with a modified mRNA encoded PDGF-BB at 24 hours. After 48 hours, alamarBlue was added and cultured for another 4 hours. At 24-hour of incubation, the supernatants from periodontal cells transfected with a modified compound of mRNA encoded PDGF-BB stimulated the periodontal cell proliferation. The percentage of cell proliferation in transfected cell group was greater than the control at 24 hours (FIG. 3).

This study is the first study using the mRNA technology for periodontal regeneration. The cells used in the study are periodontal ligament cells since they have high stem cell potentials. Periodontal ligament cells differentiate into a variety of cell lineages that resemble periodontal ligament and cementum, which is an important target organ for periodontal regeneration. According to this in vitro experiment, it indicated that mRNA encoding PDGB-BB stimulates target cells (periodontal ligament cells) and also effectively produces PDGF-BB in a large amount at 50,000 pg/ml. The PDGF-BB was released extracellularly at 24 hours and continuously released until 72 hours. Similar to rhPDGF-BB, the amount of protein was released at 24 hours and was continuously released until day 7 after stimulation (K. U. Zaman et al., 2006). Although there was no previous study using mRNA specific to PDGF-BB, there was a study using modified mRNA encoding BMP-2 in muscle-derived mesenchymal stem cells. (Zhang et al., 2019, Tissue Engineering Part A, 25(1-2): 131-44) This previous study showed that BMP-2 production was released at first 24 hours and continuously declined. However, cells remained producing and releasing BMP-2 up to Day 6. However, the present study that showed mRNA encoding PDGF-BB stimulates PDL cells to stimulate PDGF-BB in relatively large amount; which lasted for a longer period of time.

This in-vitro study revealed PDGF-BB releasing after stimulating PDL cells with mRNA was able to induce cell proliferation of PDL cells better than control group at 48 hours. Similar to previous study, Mailhot claimed that 20 ng/ml of recombinant PDGF-BB was able to induce PDL cell proliferation greater than the control group at 4-day period (Mailhot et al., 1996, Journal of Periodontology, 67(10): 981-5) Moreover, Zaman showed that recombinant PDGF-BB also induced PDL cell proliferation greater than the control group for a 7-day period (Zaman et al., 1999, Journal of Periodontal Research, 34(5): 244-50).

This in-vitro study using mRNA platform as a delivery system not only it stimulated high amounts of protein over a long period of time, but also the platform had no effect on cell viability of PDL cells. In contrast, studies using plasmid DNA specific to PDGF-BB gene imported into PDL cells showed significantly lower cell viability compare to the control (Plonka et al., 2017, Gene Ther, 24(1): 31-9).

In conclusion, this study shows that the use of mRNA encoding PDGF-BB can be delivered to the periodontal ligament cells and stimulate the cells to produce PDGF-BB protein. Moreover, protein produced can stimulate periodontal ligament cell proliferation without affecting cell viability. The result of this study will be useful for further studies in vivo and clinical trials to acquire the foundation of mRNA technology to restore affected periodontal organs.

### Example 2: The Use of Modified mRNA Encoding Platelet-Derived Growth Factor-BB Encapsulated in Lipid Nanoparticles as an Innovation in Periodontal Regeneration

Recent studies suggest that mRNA encoding growth factor may have potential use in tissue regeneration. Intra-cardiac injection with modified mRNA encoding human vascular endothelial growth factor-A (VEGF-A, angiogenesis growth factor) was effective in increasing capillary density, survival rate and improved heart function in myocardial infarction models in mice and pigs (Zangi et al., 2013 Nat Biotechnol. 31(10): 898-907; Carlsson et al., 2018 Molecular Therapy: Methods & Clinical Development Vol. 9:330). Clinical study of VEGF-A mRNA for treatment of heart failure is ongoing. mRNA encoding other growth factors has recently been used to promote bone regeneration. It was found that BMP-9 mRNA with collagen scaffold was able to effectively induce bone formation in calvarial bone defect model in rats (Khorsand et al., 2017 AAPS J. 19(2): 438-446)

Given the promising data of mRNA-regenerative therapy, experiments were conducted to explore the potential use of optimized m1Ψ-modified mRNA encoding PDGF-BB for periodontal tissue regeneration. Here, the effectiveness of PDGF-BB mRNA was evaluated as mean to transfect clinically relevant target periodontal cells/tissues both in vitro and in vivo, of which protein translation, functions, and durability were assessed.

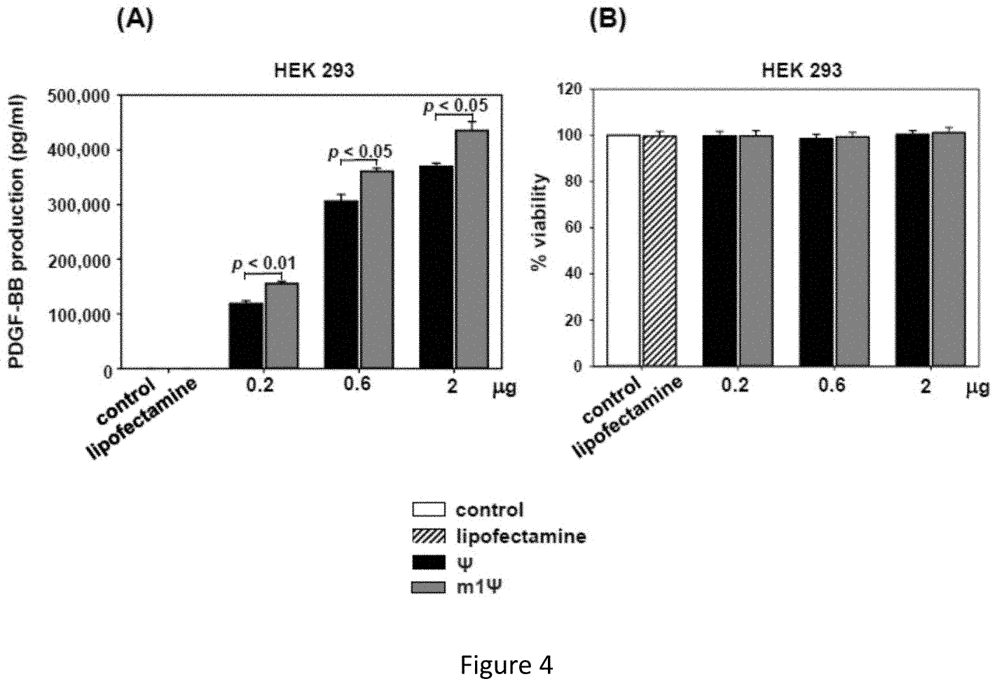

Initial experiments were conducted to evaluate the expression of PDGF-BB in transfected HEK293T (FIG. 4A and FIG. 4B). Cells at 200,000 cells/well were transfected with varying concentrations (0.2, 0.6, and 2 μg) of pseudouridine (TriLink) and N1-methylpseudouridine (m1Ψ)-modified PDGF-BB mRNA complexed with lipofectamine 2000. Production of protein PDGF-BB in culture supernatants at 48 h after transfection was assessed by ELISA. The results showed that m1Ψ modification was efficient in secreting hPDGF-BB protein and the level was significantly higher than modification. The protein translation was dose dependent and high levels of the secreted PDGF-BB protein ranging from 155,040±4,410 pg/ml to 435,467±15,949 pg/ml were detected in 48 hr-culture supernatants of m1Ψ-modified PDGF-BB mRNA transfected HEK 293T. (FIG. 4A). No PDGF-BB protein was detected in the control cells. Cell transfection did not affect the viability of periodontal cells (FIG. 4B).

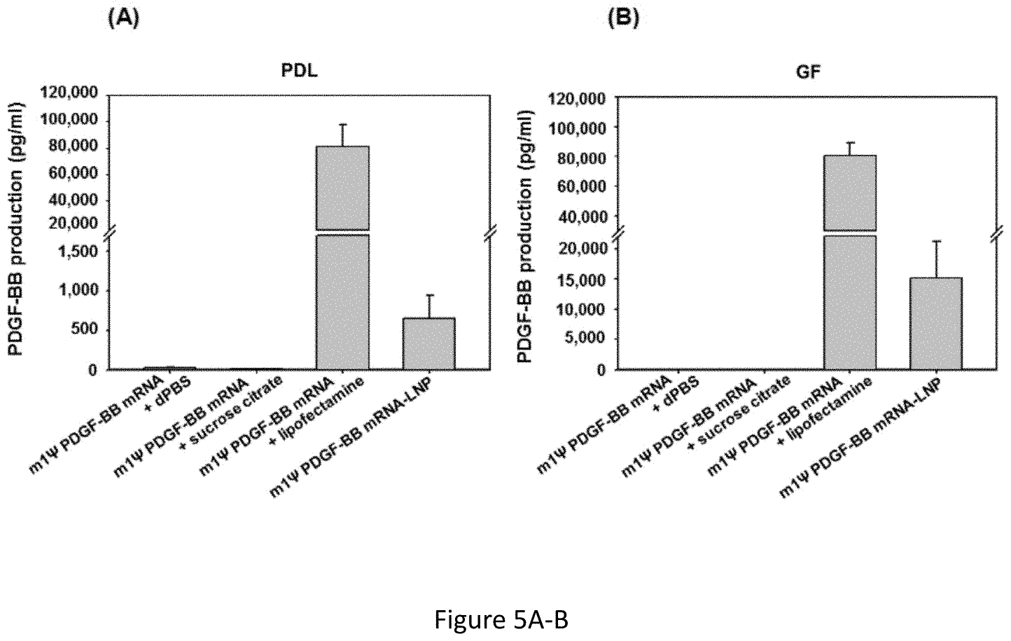

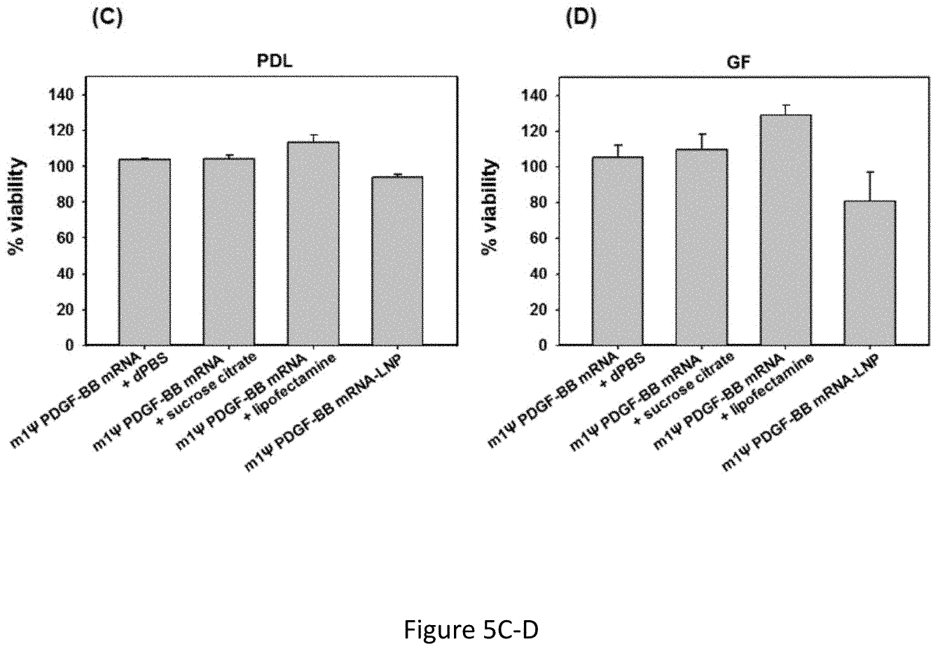

Experiments were also conducted to examine in vitro secretion of PDGF-BB protein in clinically relevant target cells after transfection with PDGF-BB mRNA formulated with different vehicles. Human periodontal ligament cells (PDLCs) and gingival fibroblasts (GFs) at 100,000 cells/well were transfected with 2 μg of PDGF-BB mRNA in dPBS, sucrose citrate buffer, lipofectamine 2000 and LNP. After 48 h of cell transfection, culture supernatants were harvested and measured for PDGF-BB by ELISA. The most efficient protein expression was detected in lipofectamine 2000 formulation with high mean protein levels of 53,246±9,344 pg/ml in PDLCs (FIG. 5A) and 49,213±9,845 pg/ml in GFs (FIG. 5B). A significant lower mean protein level (6-15 fold) was observed in LNP formulation (3,438±92 pg/ml in PDLCs and 7,817±430 pg/ml in GFs). Negligible protein translation was detected in other vehicle formulations (dPBS and sucrose citrate buffer). In vitro transfection with PDGF-BB mRNA formulated in lipofectamine 2000, dPBS and sucrose citrate buffers showed no cytotoxicity against PDLCs and GFs, however, a reduced viability of PDLCs (FIG. 5C) and GFs (FIG. 5D) to 93% and 80%, respectively was observed in LNP formulation.

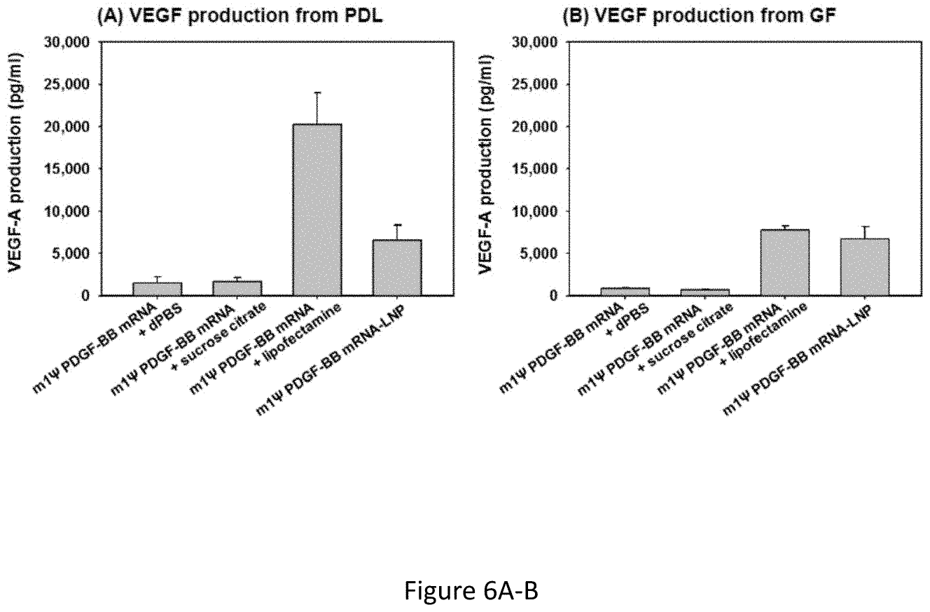

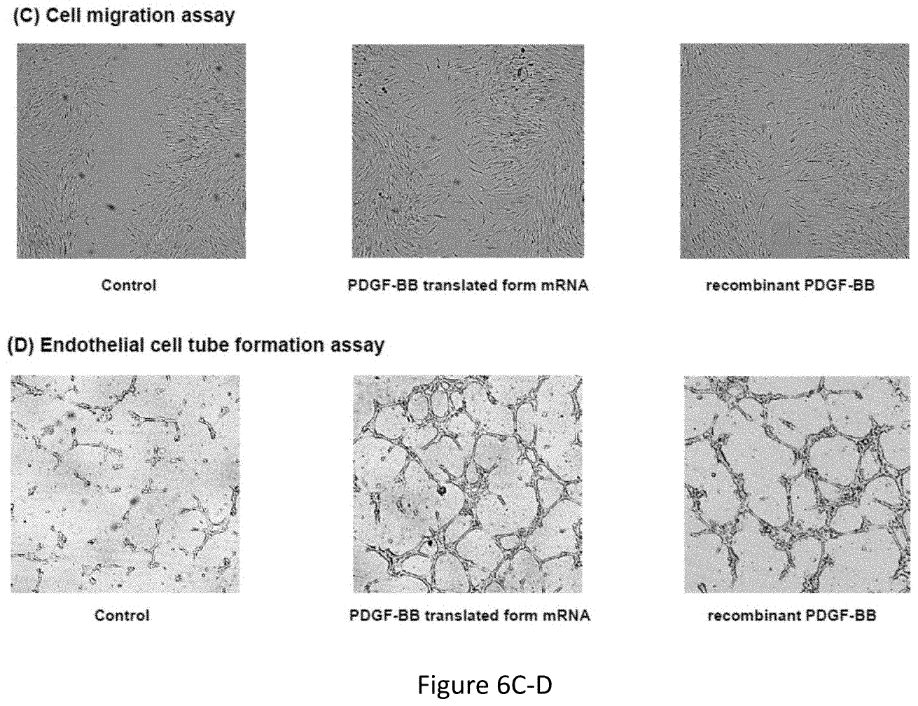

Further experiments were conducted to examine the biological function of PDGF-BB protein translated from mRNA. Transfection of PDLCs and GFs with PDGF-BB mRNA led to endogenous production of VEGF-A from both target cells, supporting previous studies that PDGF-BB can induce expression of VEGF-A (FIG. 6A and FIG. 6B). PDGF-BB secreted from mRNA transfected cells was compared with the same concentration of recombinant PDGF-BB (5 ng/ml) and found to enhance cell migration (FIG. 6C) and induce angiogenesis using endothelial cell tube formation assay (FIG. 6D).

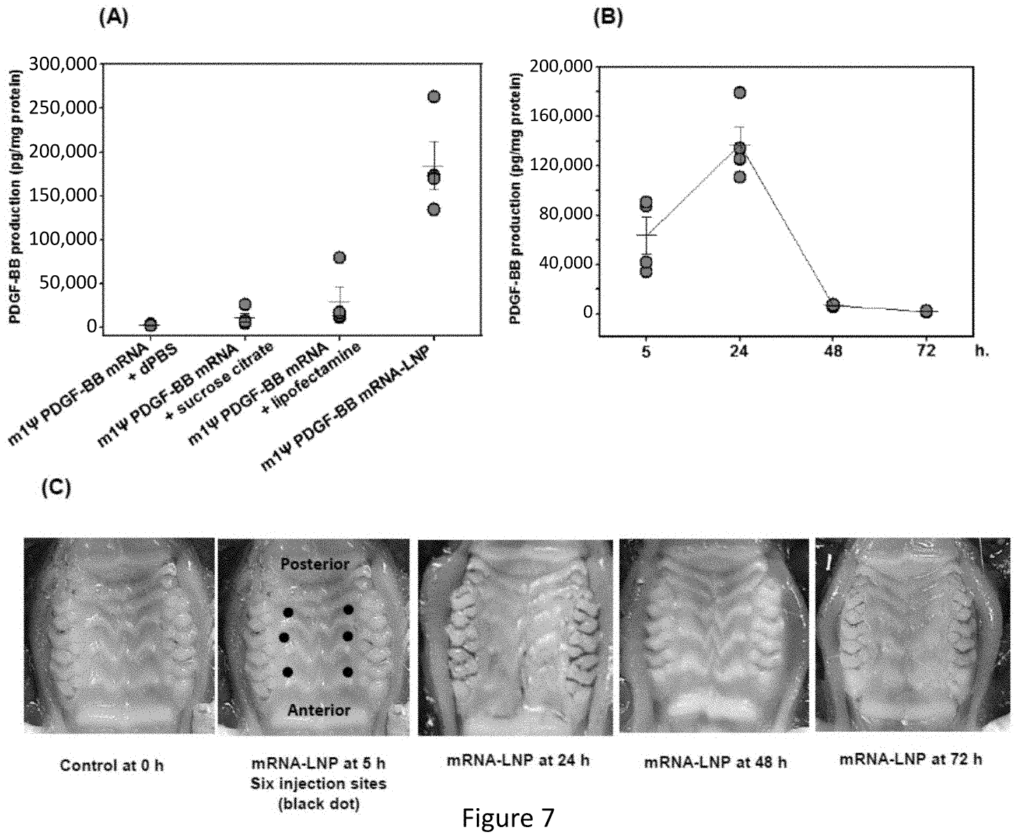

To determine translation efficient of mRNA in vivo, modified PDGF-BB mRNA formulated with dPBS, sucrose citrate buffer, lipofectamine 2000 and LNP was directly injected into rat gingiva (palatal side) (total 30 μg; 6 sites, 5 μg/6 μl/site) (FIG. 7C). Whole gingival tissues of the palate were harvested at 24 h after injection, digested with RIPA+proteinase inhibitor and measured for PDGF-BB protein production by ELISA. Twenty four hours post-injection, robust high expression of PDGF-BB was detected in gingival tissues of rats injected with PDGF-BB mRNA formulated with LNP (184,631±27,464 pg/mg protein). Whereas PDGF-BB mRNA formulated with lipofectamine 2000, which resulted in high protein expression in vitro, yielded about 6 fold lower protein expression in rat gingiva (29,665±16,536 pg/mg protein) compared to PDGF-BB mRNA formulated with LNP (FIG. 7A).

FIG. 7B shows the time course of PDGF-BB protein production after PDGF-BB mRNA-LNP injection into rat gingiva. PDGF-BB mRNA formulated with LNP was injected into rat gingiva (palate) (total 30 μg; 6 sites, 5 μg/6 μl/site). The whole gingival tissues of the palate were harvested at 5, 24, 48 and 72 h and then assessed for PDGF-BB production by ELISA. The results showed the production of PDGF-BB was detected early at 5 h (63,542±14,868 pg/mg of protein) and peaked at 24 h (137,027±14,694 pg/mg of protein). The level of PDGF-BB protein continued to decline after 72 h, but still 66 fold higher compared to baseline control (1,869±228 versus 28±12 pg/mg of protein). Injection site reactions were daily observed. No sign of erythema and swelling was detected through the course of study (FIG. 7C). In addition, the expression of gingival tissue IL-6 was undetectable and the gingival tissue TNF-α was negligible (<100 pg/mg of protein) at all time points (data not shown).

### Example 3: Utilization of Modified mRNA Encoding Bone Morphogenetic Protein-2 for Periodontal Regeneration: An In Vitro Study

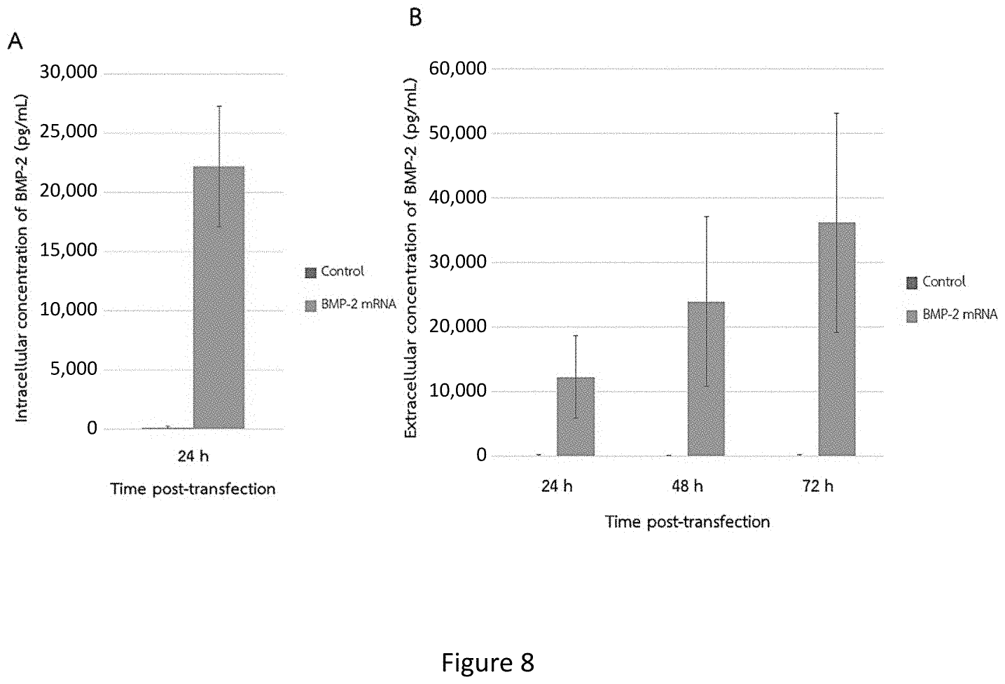

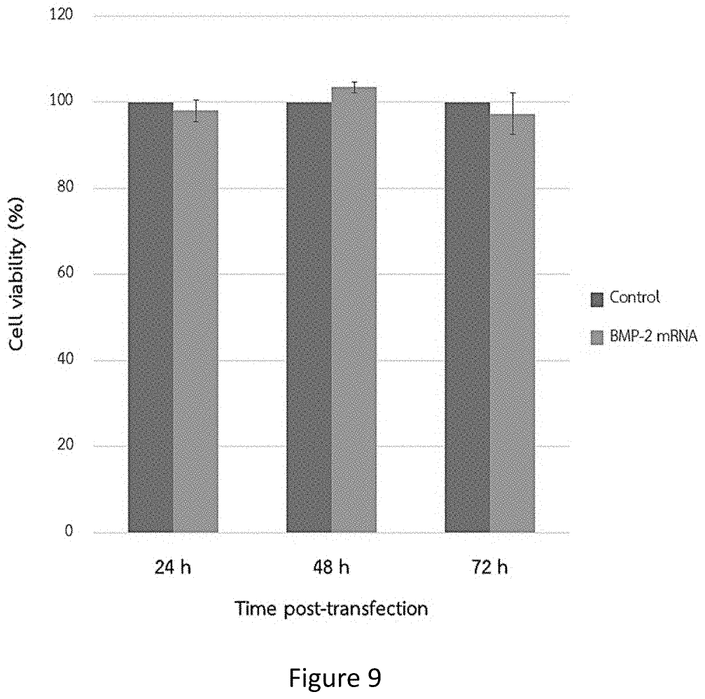

Current modalities for periodontal regeneration provide modest success, however, complete periodontal regeneration is still not achievable. Recently in vitro synthesized nucleoside-modified messenger RNA (mRNA) has emerged as a novel platform in regenerative medicine. Here, experiments were conducted to investigate the ability of human periodontal ligament cells (PDLCs), clinically relevant target cells, to produce bone morphogenetic protein-2 (BMP-2), a significant protein for bone formation after transfection with modified mRNA that encode this protein. Experiments were conducted to investigate the biological activity of translated protein in enhancing PDLC proliferation. Isolated PDLCs from healthy periodontal tissue were transfected with N1-methylpseudouridine-modified mRNA encoding BMP-2 (m1Ψ-BMP-2 mRNA) complexed with transfecting agent, Lipofectamine 2000. Cell lysates and supernatants were collected at 24, 48 and 72 hours (h) after transfection for protein production by ELISA and cell viability by alamarBlue assay. High levels of BMP-2 production were detected intracellularly and extracellularly (FIG. 8A and FIG. 8B). Secreted BMP-2 gradually increased up to 72 h (FIG. 8B). Cell viability was maintained above 90% throughout the observation period (FIG. 9). In conclusion, transfection of PDLCs with N1-methylpseudouridine-modified mRNA encoding BMP-2 in Lipofectamine 2000 led to high levels of functional BMP-2 protein. Using the in vitro synthesized nucleoside-modified mRNA may allow future application as novel therapeutics platform for periodontal regeneration.

mRNA technology is a new and highly innovated method that is safe and provide cost-effective benefits. Recently, nucleoside-modified mRNA has been emerged as a novel alternative in the non-viral gene therapy. One of the major drawbacks of mRNA in tissue regeneration is its ability to elicit innate immune response leading to an undesirable inflammatory reaction. Recently, modification of mRNA in the base region has provided the ability to evade toll-like receptors recognition that leads to inhibition of type 1 interferon production. The modification of the base region to pseudouridine or N-1 methylpseudouridine are also effective in inhibiting the innate immune response and enhances the production of proteins (Karikó et al., 2008, Molecular Therapy, the journal of the American Society of Gene Therapy, 16(11): 1833-40; Andries et al., 2015, Journal of Controlled Release: official journal of the Controlled Release Society, 217: 337-44). Furthermore, the decontamination of double stranded RNA, which was generated during in vitro synthesis, with liquid chromatography will further inhibit the innate immune response and enhance protein production.

The method of mRNA delivery into target cells is of great importance for effective production of desired proteins in vitro or in vivo. As mentioned before, mRNA can be degraded by nucleases found in most tissue. Thus, the need for an effective delivery system is crucial for preventing degradation and enhancing the protein production. The encapsulation of mRNA increases the stability of cellular uptake and endosomal escape after entering the target cell. The most widely studied method of mRNA delivery into cells indicating good outcome is cationic lipid nanoparticles. In general, cationic lipid which is positively charged will engage with the negatively charged mRNA forming lipid nanoparticles. These nanoparticles have been shown to successfully delivered mRNA into target cells in vivo.

Bone morphogenetic proteins (BMPs) are multifunctional cytokines belonging to the TGF-β superfamily which comprised of approximately 50 genes (Chen et al., 2004, Growth Factors (Chur, Switzerland), 22(4): 233-41). The roles of BMPs have been extensively studied in the areas of embryonic development and their effects on cellular functions such as growth, differentiation and apoptosis. Recent studies have revealed that BMP signals the proliferation and differentiation of chondrocytes, differentiation of mesenchymal stem cells into osteoblasts and controls bone quality (Chen et al., 2004, Growth Factors (Chur, Switzerland), 22(4): 233-41; Carreira et al., 2015, Vitamins and Hormones, 99: 293-322). In addition, BMP-2 also promotes formation of new blood vessels or angiogenesis through the production of vascular endothelial growth factor A (Deckers et al., 2002, Endocrinology, 143(4): 1545-53).

The therapeutic abilities of BMP-2 have been evaluated in various clinical settings such as calvarial, mandibular, and cleft palate reconstruction; alveolar augmentation; dental implant fixation; and for endodontic and periodontal conditions (Lindholm et al., 1996, Tissue Engineering Intelligence Unit. Bone Morphogenetic Proteins: Biology, Biochemistry, and Reconstructive Surgery. Georgetown Tex., RG Landes Company. 135-45; Wikesjo et al., 2000, J Parodontol Implantol Orale, 19: 433-457). Early studies reported the effect of applying recombinant BMP-2 in a polylactic acid-polyglycolic acid copolymer carrier into dog intrabony periodontal defects promoted significantly greater regeneration of alveolar bone and cementum.

The present experiments were conducted to develop a new highly innovated therapeutic platform of mRNA encoding BMP-2 that is highly efficient, safe and cost effective. Experiments were conducted to investigate the ability of human periodontal ligament cells (PDLCs) to produce or secrete BMP-2 after being transfected with mRNA encoding BMP-2 (m1Ψ-BMP-2 mRNA) and to test the function of produced BMP-2 as measured by cell proliferation ability and the ability to induce new blood vessels using endothelial tube formation assay in vitro.

The methods and materials employed in these experiments are now described.

Production of mRNA Encoding BMP-2

Nucleotide sequences of human BMP-2 were designed. N1 methylpseudouridine-modified mRNA was synthesized (Pardi et al., 2017, Nature Communications, 8: 14630; Pardi et al., 2018, Nature Communications, 9(1): 3361; Pardi et al., 2018, Nature Reviews Drug Discovery, 17(4): 261-79).

### Cells Isolation and Culture

Human periodontal ligament cells (PDLCs) were obtained from healthy periodontal patients (age 15-35 years) undergoing extraction of third molars for orthodontic or therapeutic reasons. PDLCs were obtained from the tooth by enzyme-digestion method. Briefly, the teeth were extensively washed twice with Dulbecco's phosphate-buffered saline (DPBS) and the PDL tissues were scraped out from the middle third of the root under sterile condition. Care was exercised to avoid contamination from gingival or pericapical granulation tissues. Then, PDL tissues were minced into fragment of 1-2 mm² and digested with a solution of 2 mg/ml collagenase and 2 mg/ml dispase for 60 minutes at 37° C. and then filter through a 70 μm cell strainer. The pass-through was then washed twice with culture medium. The PDLCs were cultured with culture medium (Alpha MEM) at 37° C. in humidified atmosphere of 5% CO₂ in air. Culture medium was changed twice a week. After 80% confluent monolayer of cells were reached, PDLCs were trypsinized, washed and then sub-cultured to new tissue culture flasks. The cells from 3^{rd} to 8^{th} passages from 3 different donors were used in this study (Iwata et al., 2010).

### In Vitro Cell Transfection and Production/Secretion of BMP-2 Protein

Human periodontal ligament cells (PDLCs) were plated in 12 wells plate (100,000 cells per well). These cells were then transfected with m1Ψ-BMP-2 mRNA complexed with Lipofectamine® 2000 (Invitrogen) according to manufacturer's instructions. To analyze the BMP-2 protein expression and secretion levels, the transfected cells and control cells (Lipofectamine only) were cultured for 24-72 hrs. Supernatants and cells were harvested at 24, 48 and 72 h time-point. and were used for analysis. Harvested cells were lysed using RIPA buffer solution (Pierce® RIPA buffer, ThermoFisher Scientific) and the lysate was collected for further analysis. Monoclonal antibodies specific to BMP-2 were used to determine the protein production and secretion by using ELISA (Quantikine®, R&D Systems).

### Cell Proliferation Ability

To examine the ability of secreted BMP-2 to enhance PDLCs proliferation, PDLCs were plated in 96 wells plate (3000 cells per well) and either control medium or supernatant of PDLCs that has been transfected with m1Ψ-BMP-2 mRNA complexed with Lipofectamine 2000. After 48 hours of incubation, 10% Alamar Blue solution (alamarBlue®, BIO-RAD) was added. After 2-4 hours, cell proliferation ability was measured by a microplate reader at absorbance of 570 nm.

### Cell Toxicity Analysis

To analyze cell toxicity, PDLCs transfected with either m1Ψ-BMP-2 mRNA complexed with Lipofectamine 2000, Lipofectamine 2000 alone or control medium were cultured with 10% alamarBlue solution (alamarBlue®, BIO-RAD) then incubated at 37° C. in humidified atmosphere of 5% CO₂. After 1-4 hours, cell culture supernatants were measured at absorbance of 570 nm using microplate reader.

The results of the experiments are now described.

Analysis of BMP-2 Production after Transfection with m1Ψ-BMP-2 mRNA

PDLCs were transfected with m1Ψ-BMP-2 mRNA complexed with Lipofectamine 2000. PDLCs transfected with Lipofectamine 2000 alone were used as controls. At each time-point of 24, 48 and 72 h, supernatants and cell lysate were collected.

After 24 h of transfection, high levels of BMP-2 were observed intracellularly in the experimental group with the mean concentration of 22,188 picogram per milliliter (pg/ml). Whereas in the control, the amount of BMP-2 production was rather low and was unable to be detected by an ELISA. As shown in FIG. 8A, PDLCs from the experimental group produced higher amounts of intracellular BMP-2 than cells from the control group.

High extracellular concentration of BMP-2 was observed in supernatants collected from the transfected cells. The BMP-2 concentration was gradually increase from each time-point with the mean concentration of 12,285; 23,964; and 36,162 pg/ml respectively. Whereas in control, the concentration was low and was hardly detected by an ELISA. As shown in FIG. 8B, extracellular concentration of BMP-2 was high and gradually increased up to 72 h in the experimental group compared to control group.

Cell Viability after Transfection with m1Ψ-BMP-2 mRNA

At each time-point of observation, alamarBlue solution was added to the transfected cells culture. After 4 h, the cell viability was analyzed using a microplate reader. As shown in FIG. 9, m1Ψ-BMP-2 mRNA and Lipofectamine 2000 had modest effects on PDLC viability. The overall percentage of viability was maintained above 90% throughout the observational period.

### Biological Activity of Translated BMP-2 Protein

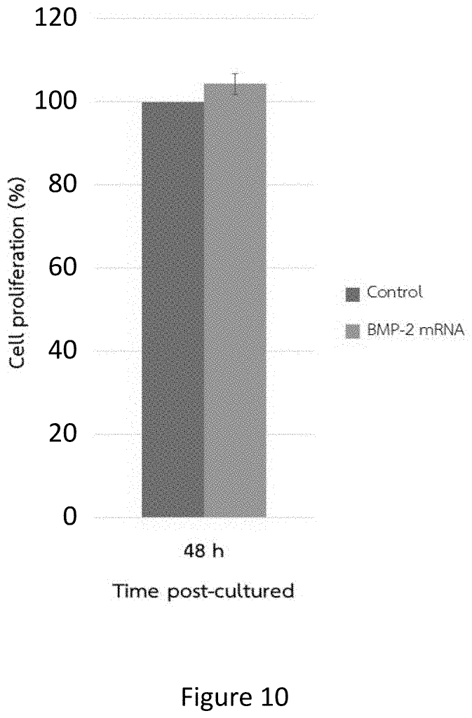

Supernatants collected at 24 h from the experimental group and control group were added to a fresh PDLC culture. After 48 h of incubation, the media were removed and 10% alamarBlue solution were added. Cell proliferation ability was measured after 4 h using a microplate reader. As shown in FIG. 10, the supernatants from the experiment group were able enhance cell proliferation but not markedly significant from control.

This study, for the first time, demonstrated the transfection ability of PDLCs with m1Ψ-BMP-2 mRNA and Lipofectamine 2000 that leads to high levels of functional BMP-2 production without an effect on cell viability. These proteins were able to induce PDLC proliferation and also promoted endothelial cell tube formation, marker of angiogenesis.

High intracellular and extracellular production of BMP-2 were observed in this study. Previous studies that transfected rat muscle derived stem cells with mRNA encoding BMP-2 demonstrated a much lower concentration of BMP-2 compared to the present study (Zhang et al., 2019, Tissue Engineering Part A, 25(1-2): 131-44). The peak concentration was observed at 24 h followed by a gradual decrease, but in this study the concentration increased gradually with highest concentration at 72 h. Also, protein production from the previous study was observed until day 6 which when compared to gradual proteins production in the present study may suggest similar period of proteins production.

Cell viability and enhancement of cell proliferation is an important factor for the demonstration of the safety and efficacy of the biomaterials especially the one that will be used for tissue regeneration in human. In this study, the nucleosides modified m1Ψ-BMP-2 mRNA and Lipofectamine 2000 had modest effects on the cell viability suggesting adequate safety for the application in human. Furthermore, the supernatants from the transfected cells can induce new PDLC proliferation. Similar results were observed in the previous study that transfected PDLCs with recombinant BMP-2 gene. Higher proliferation ability was observed in the transfected cells compared to controls (Jian et al., 2017, Biosci Rep. 37(3): BSR20160585).

In the past, recombinant BMP-2 has been use mainly in the oral surgery field. Attempts were made for the application in periodontal regeneration but there were some drawbacks. Animal studies demonstrated high amount of bone regeneration in horizontal and furcal defects in beagle dogs. However, root resorption and ankylosis at some area of the root surface were encountered. These side effects may be due to the application of high concentration of BMP-2 proteins at once, leading to extensive bone formation that exceeds the rate of cementum or PDL formation. In contrast, mRNA technology allows slow release of mRNA from the carrier that results in gradual proteins production and release. Also, mRNA can be degraded overtime, so the protein production will not be prolonged. Furthermore, combination of multiple mRNA encoding different type of proteins that promotes soft and hard tissues formation simultaneously may be used to reduce or prevent the unwanted side effects.

During 2007, the U.S. FDA approved recombinant BMP-2 as an alternative to autogenous bone graft for maxillary sinus augmentation and alveolar ridge augmentation after tooth extraction. Previous studies had shown positive results mostly adequate augmentation for implant placement, histologically indifferent form host bone and had no effects on implant survival. Nevertheless, these products are recombinant proteins with high production costs that leads to high treatment cost and limited the access for general population to receive treatment. In this case, the use of mRNA therapeutic platform may allow access for patients with reduction in cost but remain high in treatment efficacy.

Studies in past have shown that mRNA can elicit the innate immune response that resulted in undesirable inflammation and poor protein formation; however, this problem had been overcome by the modification of the mRNA.

This study demonstrated the ability of PDLCs after transfection with mRNA to produce high amounts of functional proteins. The translated BMP-2 proteins were able to enhance PDL cell proliferation and endothelial cell tube formation, the marker of angiogenesis. Using the in vitro synthesized nucleoside-modified mRNA may allow future application as novel therapeutics platform for periodontal regeneration.

### Example 4: In Vivo Use of Modified mRNA Encoding Platelet-Derived Growth Factor-BB and Bone Morphogenetic Protein-2

Several growth and differentiation factors, including insulin like growth factor 1, fibroblast growth factor 2 (FGF-2), platelet-rich plasma protein, platelet-derived growth factor, and bone morphogenetic proteins (BMPs) have been employed to accomplish predictable bone formation in the fields of periodontology and oral maxillofacial surgery (Schliephake H. et al., 2002 Int J Oral Maxillofac Surg 31:469-484; Taba M. et al., 2005 Orthod Craniofac Res 8:292-302). In particular, BMP-2 has been shown to possess excellent osteoinductive activity and has been extensively studied (Herford and Boyne, 2008 J Oral Maxillofac Surg 66:616-624; Jovanovic S A et al. 2007 Clin Oral Implants Res 18:224-230). Some reports have demonstrated that the application of recombinant human BMP-2 (rhBMP-2) with an absorbable collagen sponge induces new bone formation in mandibular and cleft palate defects, with results comparable to autogenous bone grafts (Herford and Boyne, 2008 J Oral Maxillofac Surg 66:616-624).

The calvarial bone defect model with a diameter of 5 mm is considered effective for evaluating bone regenerative effects of various biomaterials and treatment modalities because of its convenience, reproducibility and relatively slight surgical invasion. This pilot study was to evaluate the effect of PDGF-BB mRNA in LNPs and BMP-2 mRNA in LNPs on new bone formation by using a rat calvarial bone defect model.

Rats (male Wistar rats) were anesthetized, and two defects of 5 mm diameter were created on the parietal bone on both sides of the sagittal suture. Seven groups of animals received: 1) collagen scaffold (Col); 2) Col loaded with 15 μg of PDGF-BB mRNA-LNP; 3) Col loaded with 5 μg of PDGF-BB mRNA-LNP and 4) Col loaded with 1.5 μg of PDGF-BB mRNA-LNP 5) Col loaded with 15 μg of BMP-2 mRNA-LNP; 6) Col loaded with 5 μg of BMP-2 mRNA-LNP and 7) Col loaded with 1.5 μg of BMP-2 mRNA-LNP (One animal per group). Both left and right defects in each animal received the same treatment. At week 4 after treatment, animals were euthanized, and the new bone formation was analyzed by microcomputed tomography (μCT).

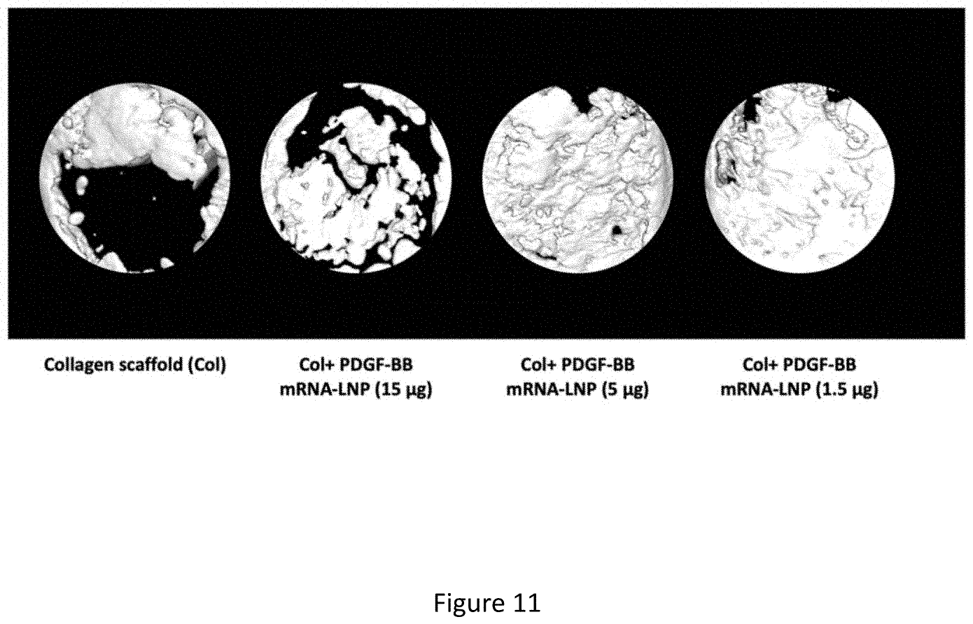

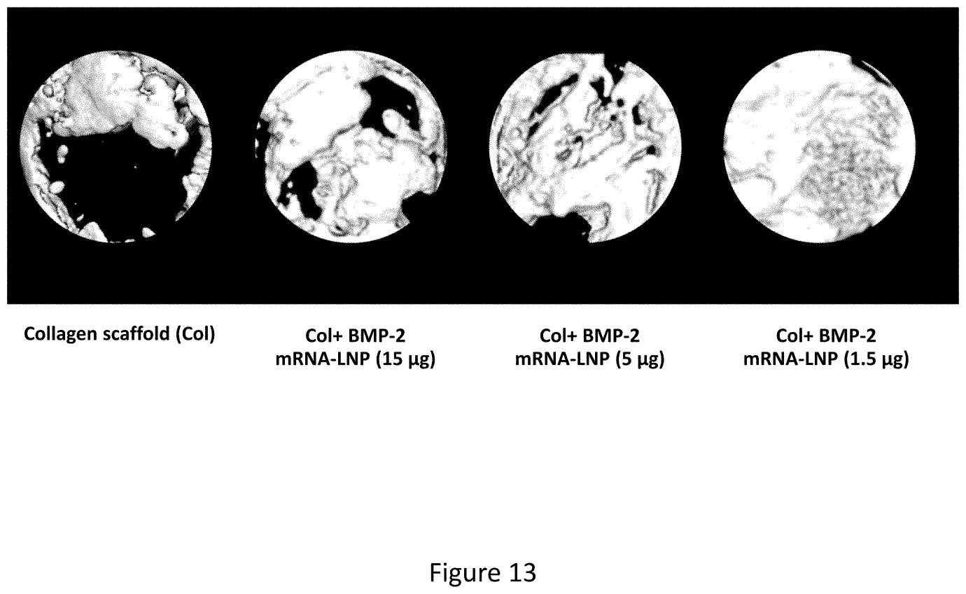

Postoperative clinical healing was uneventful in all surgical sites, except limited signs of inflammation until a few days postsurgery. No visible complications, such as material exposure, infection, or suppuration, were observed during the rest of the experimental period. 3D μ-CT images are shown in FIG. 11 for PDGF-BB mRNA treatment and FIG. 13 for BMP-2 mRNA treatment. After 4 weeks of healing, broad radiopacity area within the defects was observed in all the mRNA treatment defects as compared to those of controls (collagen scaffold only).

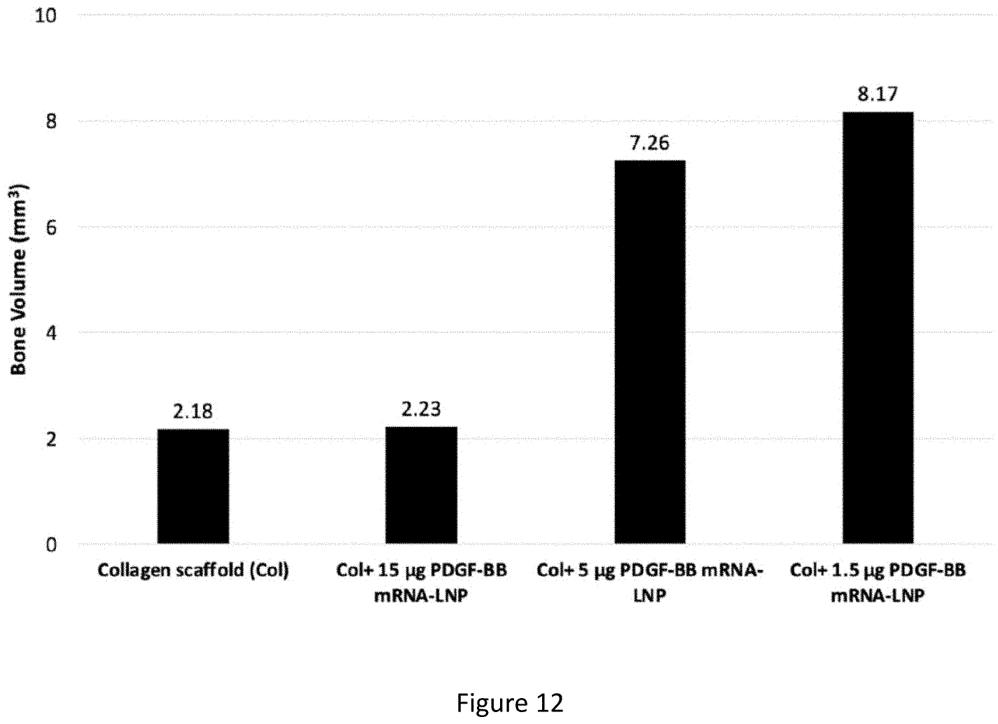

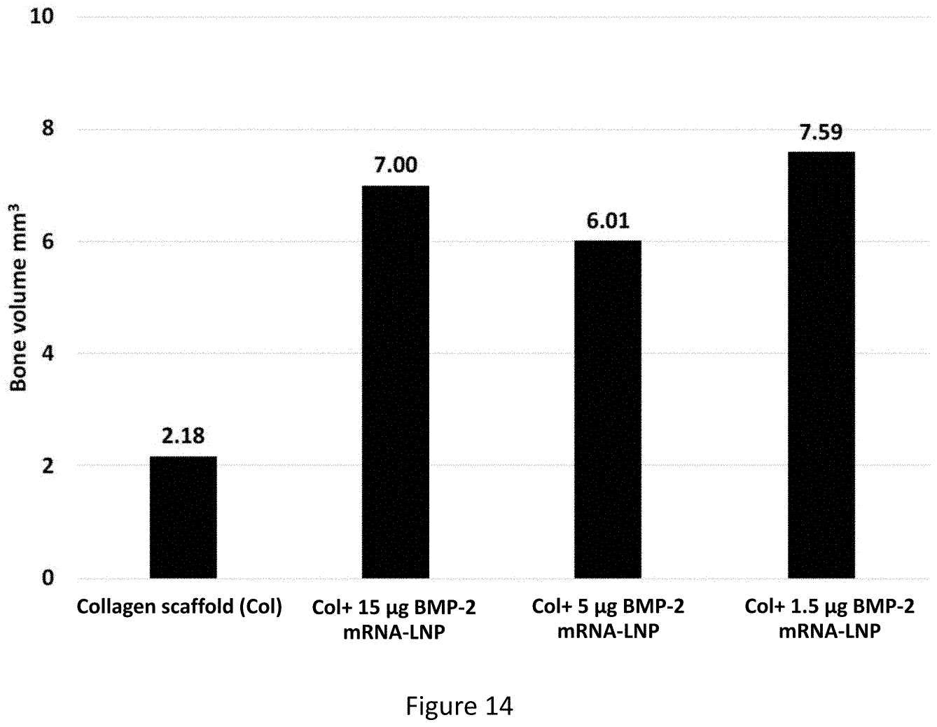

Bone volume in bone defects are shown in FIG. 12 for PDGF-BB mRNA treatment and FIG. 14 for BMP-2 mRNA treatment. Notably, bone volume in Col loaded with 5 μg and 1.5 μg of PDGF-BB mRNA had a mean bone volume about three fold higher than that of the Col loaded 15 μg PDGF-BB and Col control. For BMP-2 mRNA treatment, bone volume in Col loaded with 15 μg, 5 μg and 1.5 had a similar mean bone volume, of which about three fold higher than that of the Col control.

These results suggest that PDGF-BB mRNA-LNP and BMP-2 mRNA-LNP with collagen scaffold effectively enhance bone regeneration at 4 weeks post treatment in calvarial bone defect model in rats.

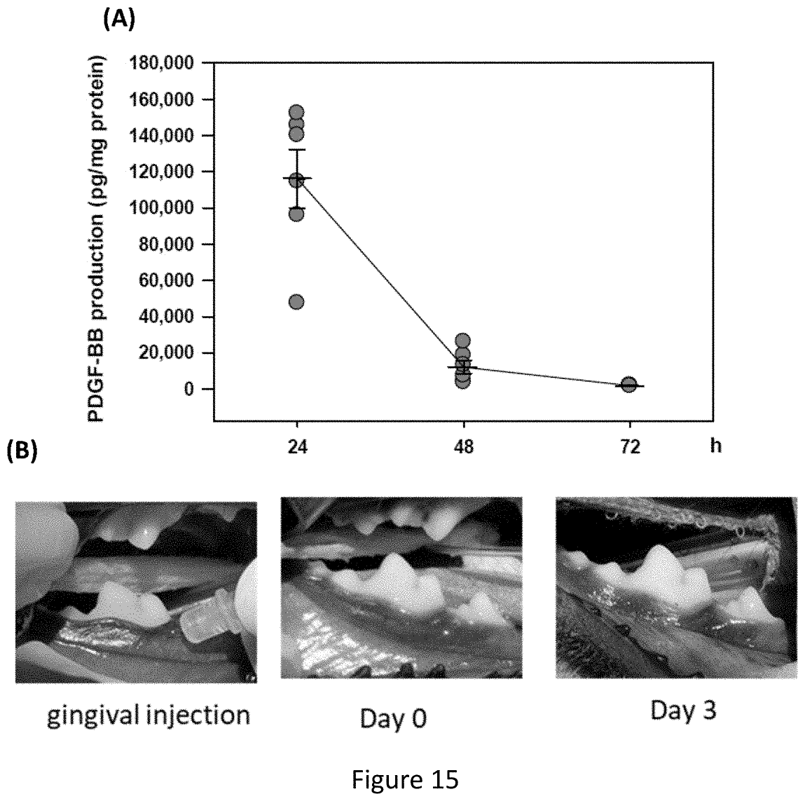

To extend the findings of in vivo protein expression studies, PDGF-BB production was evaluated in large animals (dogs), which have an alveolar bone architecture similar to humans. PDGF-BB mRNA-LNPs were directly injected into dog gingiva on the buccal side and then PDGF-BB expression was evaluated at different time points (FIGS. 15A and 15B). Similar to rat experiments, expression of PDGF-BB protein in dog peaked at 24 h after injection (116,003±16,190 pg/mg of protein) and then declined. At 72 h after injection the amount of PDGF-BB was about 3 fold higher than those injected with PBS control (1,772+64 versus 471+289 pg/mg of protein).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for inducing regeneration of periodontal tissue, bone, or a combination thereof in a subject, the composition comprising at least one isolated RNA encoding at least one growth factor, or fragment or variant thereof.

2. The composition of claim 1, wherein the at least one isolated RNA comprises at least one isolated nucleoside-modified RNA.

3. The composition of claim 2, wherein the at least one isolated nucleoside-modified RNA comprises pseudouridine.

4. The composition of claim 2, wherein the at least one isolated nucleoside-modified RNA comprises 1-methyl-pseudouridine.

5. The composition of claim 2, wherein the at least one isolated nucleoside-modified RNA is a purified nucleoside-modified RNA.

6. The composition of claim 1, wherein the at least one growth factor comprises PDGF-BB or BMP-2.

7. The composition of claim 1, further comprising a lipid nanoparticle (LNP).

8. The composition of claim 7, wherein the at least one isolated RNA is encapsulated within the LNP.

9. The composition of claim 1, wherein the composition comprises a scaffold.

10. A method of inducing regeneration of periodontal tissue, bone, or a combination thereof in a subject comprising administering to the subject an effective amount of a composition comprising at least one isolated RNA encoding at least one growth factor, or fragment or variant thereof.

11. The method of claim 10, wherein the at least one isolated RNA comprises at least one isolated nucleoside-modified RNA.

12. The method of claim 11, wherein the at least one isolated nucleoside-modified RNA comprises pseudouridine.

13. The method of claim 11, wherein the at least one isolated nucleoside-modified RNA comprises 1-methyl-pseudouridine.

14. The method of claim 11, wherein the at least one isolated nucleoside-modified RNA is a purified nucleoside-modified RNA.

15. The method of claim 10, wherein the at least one growth factor comprises PDGF-BB or BMP-2.

16. The method of claim 10, wherein the composition further comprises a lipid nanoparticle (LNP).

17. The method of claim 16, wherein the at least one isolated RNA is encapsulated within the LNP.

18. The method of claim 10, wherein the composition comprises a scaffold.

19. The method of claim 10, wherein the method comprises a single administration of the composition.

20. The method of claim 10, wherein the method comprises multiple administrations of the composition.

21. The method of claim 18, wherein the method comprises administration of the scaffold to periodontal tissue, bone defects, or a combination thereof of the subject.


SEQUENCE LISTING (4 sequences)

SEQ ID NO: 1 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 2 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 3 - DNA, 723 bp, Artificial Sequence
  PDGF-BB DNA sequence
atgaaccgctgctgggccctgttcctgtccctgtgctgctacctgcgcctggtgtccgccgagggcgaccccatccccgaggagctgtacgagatgctgtccgaccactccatccgctccttcgacgacctgcagcgcctgctgcacggcgaccccggcgaggaggacggcgccgagctggacctgaacatgacccgctcccactccggcggcgagctggagtccctggcccgcggccgccgctccctgggctccctgaccatcgccgagcccgccatgatcgccgagtgcaagacccgcaccgaggtgttcgagatctcccgccgcctgatcgaccgcaccaacgccaacttcctggtgtggcccccctgcgtggaggtgcagcggtgctccggctgctgcaacaaccgcaacgtgcagtgccgccccacccaggtgcagctgcgccccgtgcaggtgcgcaagatcgagatcgtgcgcaagaagcccatcttcaagaaggccaccgtgaccctggaggaccacctggcctgcaagtgcgagaccgtggccgccgcccgccccgtgacccgctcccccggcggctcccaggagcagcgcgccaagaccccccagacccgcgtgaccatccgcaccgtgcgcgtgcgccgcccccccaagggcaagcaccgcaagttcaagcacacccacgacaagaccgccctgaaggagaccctgggcgcc

SEQ ID NO: 4 - DNA, 1188 bp, Artificial Sequence
  BMP-2 DNA sequence
atggtggccggcacccgctgcctgctggccctgctgctgccccaggtgctgctgggcggcgccgccggcctggtgcccgagctgggccgccgcaagttcgccgccgcctcctccggccgcccctcctcccagccctccgacgaggtgctgtccgagttcgagctgcgcctgctgtccatgttcggcctgaagcagcgccccaccccctcccgcgacgccgtggtgcccccctacatgctggacctgtaccgccgccactccggccagcccggctcccccgcccccgaccaccgcctggagcgcgccgcctcccgcgccaacaccgtgcgctccttccaccacgaggagtccctggaggagctgcccgagacctccggcaagaccacccgccgcttcttcttcaacctgtcctccatccccaccgaggagttcatcacctccgccgagctgcaggtgttccgcgagcagatgcaggacgccctgggcaacaactcctccttccaccaccgcatcaacatctacgagatcatcaagcccgccaccgccaactccaagttccccgtgacccgcctgctggacacccgcctggtgaaccagaacgcctcccgctgggagtccttcgacgtgacccccgccgtgatgcgctggaccgcccagggccacgccaaccacggcttcgtggtggaggtggcccacctggaggagaagcagggcgtgtccaagcgccacgtgcgcatctcccgctccctgcaccaggacgagcactcctggtcccagatccgccccctgctggtgaccttcggccacgacggcaagggccaccccctgcacaagcgcgagaagcgccaggccaagcacaagcagcgcaagcgcctgaagtcctcctgcaagcgccaccccctgtacgtggacttctccgacgtgggctggaacgactggatcgtggccccccccggctaccacgccttctactgccacggcgagtgccccttccccctggccgaccacctgaactccaccaaccacgccatcgtgcagaccctggtgaactccgtgaactccaagatccccaaggcctgctgcgtgcccaccgagctgtccgccatctccatgctgtacctggacgagaacgagaaggtggtgctgaagaactaccaggacatggtggtggagggctgcggctgccgc